(19) United States
(12) Patent Application Publication
(10) Pub. No.: US 2024/0139251 A1
(43) Pub. Date: May 2, 2024

(54) ANTIGEN-SPECIFIC T CELLS, METHODS OF PRODUCING THE SAME, AND USES THEREOF

(71) Applicant: CYTOARM CO., LTD, TAIPEI CITY (TW)

(72) Inventors: Kuo-Hsiang Chuang, Taipei City (TW); Yi-Jou Chen, Keelung City (TW); Michael Chen, Hsinchu County (TW)

(21) Appl. No.: 18/542,658

(22) Filed: Dec. 16, 2023

Related U.S. Application Data
(63) Continuation-in-part of application No. 16/497,805, filed on Sep. 26, 2019, now Pat. No. 11,913,025.
(60) Provisional application No. 62/478,280, filed on Mar. 29, 2017.

(51) Int. Cl. A61K 35/17; A61K 39/00; A61P 35/00; C07K 16/28; C12N 5/0783

(52) U.S. Cl. CPC A61K 35/17; A61K 39/4611; A61K 39/464404; A61K 39/464411; A61P 35/00; C07K 16/2809; C07K 16/2863; C12N 5/0636

(57) ABSTRACT

Disclosed herein are methods of producing tumor antigen-specific T cells and uses thereof. In the present method, peripheral blood mononuclear cells (PBMCs) isolated from a subject are cultivated with bi-specific antibodies (BsAbs) in a culture medium so as to produce the tumor antigen-specific T cells from the PBMCs. Also provided in the present disclosure are tumor antigen-specific T cells produced by the present method, and uses thereof in treating subjects suffering from cancers.

### CROSS-REFERENCE OF RELATED APPLICATION

This application is a continuation-in-part of U.S. Application No. 16,497/805, filed Sep. 26, 2019, which is a U.S. National Stage Filing under 35 U.S.C. 371 from International Patent Application Serial No. PCT/CN2018/081084, entitled “ANTIGEN-SPECIFIC T CELLS AND USES THEREOF”, filed Mar. 29, 2018, and published on Oct. 4, 2018. The PCT application claims priority to US. Application No. 62/478,280, filed on Mar. 29, 2017. The contents of these applications are incorporated herein by reference in their entirety.

### SEQUENCE LISTING XML

The present application is being filed along with a Sequence Listing XML in electronic format. The Sequence Listing XML is provided as an XML file entitled P3096-1-US_SEQ_AF, created Dec. 8, 2023, which is 244 Kb in size. The information in the electronic format of the Sequence Listing XML is incorporated herein by reference in its entirety.

### BACKGROUND OF THE INVENTION

### 1. Field of the Invention

The present disclosure relates to treatments of cancers. Specifically, the present disclosure relates to antigen-specific T cells and their uses for suppressing the growth or metastasis of cancers.

### 2. Description of Related Art

T cells are of a rare cell population, in which merely 5 to 10% of CD4⁺ T cells are found in peripheral blood. Proliferation of T cells can be promoted by stimulating with cytokines, such as IL-2, IL-4, and IL-5, in addition to anti-CD28 antibodies. However, the level is still insufficient for any clinical applications, such as application that involves increasing the number of activated T cells and transferring them back to human (e.g., chimeric antigen receptor (CAR) T cell therapy).

One way to activate T cell or to induce differentiation of T cell is via activating signal transduction pathway mediated by CD3 using anti-CD3 antibodies. To date, the activation of human T cells via the CD3 antigen complex have been carried out with mouse anti-human IgG2a isotype (OKT3) monoclonal antibody, which induces a mitogenic response equal to that of concanavalin A (Con A). However, one major concern of a non-human origin monoclonal antibody (e.g., murine OKT3 Ab) is its immunogenicity to the recipient, in some cases, caused dangerous allergic reactions. Human T cells activated by murine OKT3 Abs inevitably carry murine protein fragments on the surfaces, thereby posting a potential threat to their recipient.

In view of the foregoing, there remains in the related field a need of less immunogenic antibodies that may replace murine OKT3 antibodies, and a need of an improved method of activating human T cells without using murine OKT3 antibodies.

### SUMMARY

The present disclosure provides T cells differentiated and proliferated by BsAbs of the present disclosure, and their uses for treating cancers.

Accordingly, it is the first objective of the present disclosure to provide a method of inducing differentiation and/or proliferation of T cells from peripheral blood mononuclear cells (PBMCs), i.e., producing tumor antigen-specific T cells from PBMCs. The method includes, culturing PBMCs with bi-specific antibodies (BsAbs) in a culture medium so as to induce the proliferation and/or differentiation of T cells from the PBMCs, in which each BsAbs comprises a tumor antigen binding site and a CD3 binding site. According to the embodiments of the present disclosure, the BsAbs are not murine OKT3 antibodies.

According to embodiments of the present disclosure, each BsAbs is in the structure of single chain variable fragment (scFv), an antigen-binding fragment (Fab), a F(ab′)₂ or an IgG.

According to embodiments of the present disclosure, the tumor antigen binding site of each BsAbs binds to any of epidermal growth factor receptor (EGFR), programmed cell death-ligand 1 (PD-L1), or prostate specific membrane antigen (PSMA).

According to optional embodiments of the present disclosure, the culture medium may further comprise a cytokine selected from the group consisting of IL-2, TGF-β, or a combination thereof. In one preferred embodiment, the culture medium comprises both of the IL-2 and the TGF-β, and the T cells thus formed are regulator T cells.

According to some preferred embodiments of the present disclosure, the culture medium comprises IL-2, and does not comprise TGF-β. In these embodiments, the T cells thus formed are CD3⁺ and CD8⁺ T cell (also designated as “CD3⁺/CD8⁺ T cell” or “CD3⁺CD8⁺ T cell”, i.e., the T cell having CD3 and CD8 expressed on its surface).

According to embodiments of the present disclosure, in each BsAbs, the tumor antigen binding site comprises a tumor antigen scFv at least 90% identical to any of SEQ ID NOs: 69, 77, 84, 90, 96, 102, 108, 114, or 120; and the CD3 binding site comprises an anti-CD3 VL-Ck domain at least 90% identical to SEQ ID NO: 67, and an anti-CD3 VH-CH1 domain at least 90% identical to SEQ ID NO: 68.

According to embodiments of the present disclosure, in each BsAbs, the tumor antigen binding site comprises an anti-tumor antigen scFv at least 90% identical to any of SEQ ID NOs: 70, 78, 85, 91, 97, 109, or 121; and the CD3 binding site comprises an anti-CD3 scFv at least 90% identical to SEQ ID NOs: 66 or 79.

According to embodiments of the present disclosure, in each BsAbs, the tumor antigen binding site comprises an anti-tumor antigen VL-Ck domain at least 90% identical to SEQ ID NOs: 64, 75, 82, 88, 94, 100, 106, 112, or 118; and an anti-tumor antigen VH-CH1 domain at least 90% identical to SEQ ID NOs: 65, 76, 83, 89, 95, 101, 107, 113, or 119; and the CD3 binding site comprises an anti-CD3 scFv at least 90% identical to SEQ ID NO: 66 or 79.

According to embodiments of the present disclosure, in each BsAbs, the tumor antigen binding site comprises an anti-tumor antigen VL-Ck domain at least 90% identical to SEQ ID NOs:73, 80, 86, 92, 98, 104, 110, 116 or 122, and an anti-tumor antigen VH-CH1-Fc domain at least 90% identical to SEQ ID NOs: 74, 81, 87, 93, 99, 105, 111, 117, or 123; and the CD3 binding site comprises an anti-CD3 VL-Ck domain at least 90% identical to SEQ ID NO: 71, and an anti-CD3 VH-CH1-Fc domain at least 90% identical to SEQ ID NO: 72.

In practice, antigen-specific T cells prepared in accordance by the present method may be mixed with the humanized BsAb described above to form a mixture, and the mixture is then administered to a subject in need of a treatment of cancer.

Accordingly, it is the third objective of the present disclosure to provide a pharmaceutical kit for the treatment of cancer. The pharmaceutical kit comprises, the T cells differentiated and proliferated in accordance with the present method, and a humanized BsAb of the present disclosure.

It is the fourth objective of the present disclosure to provide a method of treating a subject afflicted with a cancer. The method includes the step of, administered to the subject an effective amount of the T cells prepared by the method described above, or an effective amount of a murine OKT3 T cell modified with the present BsAb.

According to some embodiments, the murine OKT3 T cell are modified to arm with the BsAb of the present disclosure on its surface by cultivating with the present BsAb in a culture medium. In addition or optionally, the culture medium may further comprise a cytokine selected from the group consisting of IL-2, IL-7, and a combination thereof.

Preferably, the T cells of the present disclosure are administered to the subject in the amount of 1×10⁴ to 1×10⁷ cells/Kg body weight of the subject. The amount can be administered in a single dose, or alternatively in more than one smaller doses.

Cancers, preferably those that are positive with the expression of EGFR, PSMA, or PD-L1 are treatable by the present method. Examples of the cancer treatable by the present method includes, but is not limited to, bladder cancer, biliary cancer, bone cancer, brain tumor, breast cancer, cervical cancer, colorectal cancer, colon cancer, esophageal cancer, epidermal carcinoma, gastric cancer, gastrointestinal stromal tumor (GIST), glioma, hematopoietic tumors of lymphoid lineage, hepatic cancer, non-Hodgkin's lymphoma, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, and thyroid follicular cancer.

According to one specific embodiment of the present disclosure, the cancer is triple-negative breast cancer. According to another specific embodiment of the present disclosure, the cancer is a malignant pancreatic cancer.

The details of one or more embodiments of the invention are set forth in the accompanying description below. Other features and advantages of the invention will be apparent from the detail descriptions, and from claims.

### BRIEF DESCRIPTION OF THE DRAWINGS

These and other features, aspects and advantages of the present invention will become better understood with reference to the following description, appended claims and the accompanying drawings, where:

FIG. 1 are schematic diagrams of the structures of (A) anti-antigen Fab/anti-CD3 scFv, (B) anti-CD3 Fab/anti-antigen scFv, (C) anti-CD3 scFv/anti-antigen scFv, and (D) anti-antigen knob/anti-CD3 hole BsAbs of the present disclosure;

FIG. 2A is a schematic presentation of treating a subject with antigen specific T cells produced by one-step expanding, differentiation and/or modification with BsAbs of the present disclosure;

FIG. 2B is a schematic presentation of treating a subject with murine OKT3 T cells produced by one-step modification with BsAbs of the present disclosure;

FIG. 3 is a schematic diagram of DNA constructs for the expression of BsAbs of the present disclosure;

FIG. 4 depicts the results of sodium dodecyl-sulfate polyacrylamide gel electrophoresis (SDS-PAGE) analysis of BsAbs of Example 1.2 in non-reducing condition (left panel) and reducing condition (right panel);

FIG. 5 depicts the flow cytometry analysis of the BsAbs of Example 1.2;

FIG. 6 depicts the differentiation and proliferation of T cells induced by murine OKT3 antibodies and BsAbs of Example 2 of the present disclosure;

FIG. 7 is the flow cytometry analysis of the anti-antigen fragments on the surfaces of the proliferated T cells in accordance with one embodiment of the present disclosure;

FIG. 8 depicts the cytotoxicity analysis of murine OKT3 T cells and T cells of Example 2 on prostate cancer cell line LNCaP in accordance with one embodiment of the present disclosure;

FIG. 9 are photographs depicting respective binding of murine OKT3 T cells and T cells of Example 2 on tumor cells in accordance with one embodiment of the present disclosure;

FIG. 10 are bar graphs depicting respective levels of cytokines secreted by murine OKT3 T cells and T cells of Example 2 at various effector:target ratios (E/T ratios) in accordance with one embodiment of the present disclosure;

FIG. 11 is a line graph depicting the cytotoxicity analysis of murine OKT3 T cells and T cells of Example 2 further modified by BsAbs of Example 1.2 on LNCaP cells at various E/T ratios in accordance with one embodiment of the present disclosure;

FIG. 12 are bar graphs depicting respective levels of cytokines secreted by murine OKT3 T cells and T cells of Example 2 further modified with BsAbs of Example 1.2 at various E/T ratios in accordance with one embodiment of the present disclosure;

FIG. 13 illustrates the cytotoxicity analysis of murine OKT3 T cells, T cells of Example 2, or T cells of Example 2 further modified with anti-CD3 Fab/anti-PD-L1scFv BsAb on malignant pancreatic cell line MIA PaCa-2 and triple negative breast cancer (TNBC) cell line MDA-MB-231 in accordance with one embodiment of the present disclosure;

FIG. 14A are photographs illustrating respective binding of murine OKT3 T cells and T cells of Example 2 on MIA PaCa-2 cells in accordance with one embodiment of the present disclosure;

FIG. 14B are photographs illustrating respective binding of murine OKT3 T cells and T cells of Example 2 on MDA-MB-231 cells in accordance with one embodiment of the present disclosure;

FIG. 15 illustrates the time course of residual amounts of BsAb of Example 1.2 remained on the surfaces of T cells in accordance with one embodiment of the present disclosure;

FIG. 16 illustrates the cytotoxicity analysis of murine OKT3 T cells and murine OKT3 T cells further modified with BsAbs of Example 1.2 on prostate cancer cell line LNCaP in accordance with one embodiment of the present disclosure;

FIG. 17 are bar graphs depicting respective levels of cytokines secreted from murine OKT3 T cells and murine OKT3 T cells further modified with BsAbs of Example 1 in accordance with one embodiment of the present disclosure;

FIG. 18 are photographs depicting respective binding of murine OKT3 T cells and murine OKT3 T cells further modified with BsAbs of Example 1.2 on prostate tumor cells (LNCaP cells) in accordance with one embodiment of the present disclosure;

FIG. 19A illustrates the cytotoxicity analysis of murine OKT3 T cells and murine OKT3 T cells further modified with anti-EGFR Fab/anti-CD3 scFv of Example 1.2 on HT29 cells in accordance with one embodiment of the present disclosure;

FIG. 19B are bar graphs depicting respective levels of cytokines secreted from murine OKT3 T cells and murine OKT3 T cells further modified with anti-EGFR Fab/anti-CD3 scFv of Example 1 in accordance with one embodiment of the present disclosure;

FIG. 20 are photographs depicting respective binding of murine OKT3 T cells and murine OKT3 T cells further modified with anti-EGFR Fab/anti-CD3 scFv of Example 1.2 on HT29 cells in accordance with one embodiment of the present disclosure;

FIG. 21A illustrates the cytotoxicity analysis of murine OKT3 T cells and murine OKT3 T cells further modified with anti-CD3 Fab/anti-PD-L1 scFv of Example 1.2 on LNCaP cells in accordance with one embodiment of the present disclosure;

FIG. 21B are bar graphs depicting respective levels of cytokines secreted from murine OKT3 T cells and murine OKT3 T cells further modified with anti-CD3 Fab/anti-PD-L1 scFv of Example 1.2 in accordance with one embodiment of the present disclosure;

FIG. 22 are photographs depicting respective binding of murine OKT3 T cells and murine OKT3 T cells further modified with anti-CD3 Fab/anti-PD-L1 scFv of Example 1.2 on LNCaP cells in accordance with one embodiment of the present disclosure;

FIG. 23 illustrates the cytotoxicity analysis of murine OKT3 T cells, and murine OKT3 T cells further modified with anti-CD3 Fab/anti-PD-L1scFv BsAb on malignant pancreatic cell line MIA PaCa-2 and triple negative breast cancer (TNBC) cell line MDA-MB-231 in accordance with one embodiment of the present disclosure;

FIG. 24A are photographs depicting respective binding of murine OKT3 T cells and murine OKT3 T cells further modified with anti-CD3 Fab/anti-PD-L1 scFv of Example 1.2 on MIA PaCa-2 cells in accordance with one embodiment of the present disclosure;

FIG. 24B are photographs depicting respective binding of murine OKT3 T cells and murine OKT3 T cells further modified with anti-CD3 Fab/anti-PD-L1 scFv of Example 1.2 on MDA-MB-231 cells in accordance with one embodiment of the present disclosure;

FIG. 25 illustrates the quantified fluorescent intensity in animals treated with the modified OKT3 T cells or the murine OKT3 T cells further modified with anti-EGFR Fab/anti-CD3 scFv of Example 1.2 in accordance with one embodiment of the present disclosure;

FIG. 26 illustrates the distribution pattern of the modified OKT3 T cells or the murine OKT3 T cells further modified with anti-EGFR Fab/anti-CD3 scFv of Example 1.2 in various organs of the test animal in accordance with one embodiment of the present disclosure;

FIG. 27A is a line graph depicting changes in the size of a tumor treated by BsAb of Example 1.2, murine OKT3 T cells, or murine OKT3 T cells modified with anti-EGFR Fab/anti-CD3 scFv of Example 1.2 in accordance with one embodiment of the present disclosure;

FIG. 27B is a line graph depicting changes in the body weight of the test animal treated by BsAb of Example 1.2, murine OKT3 T cells, or murine OKT3 T cells modified with anti-EGFR Fab/anti-CD3 scFv of Example 1.2 in accordance with one embodiment of the present disclosure;

FIG. 27C illustrates the weight of a tumor treated by BsAb of Example 1.2, murine OKT3 T cells, or murine OKT3 T cells modified with anti-EGFR Fab/anti-CD3 scFv of Example 1.2 in accordance with one embodiment of the present disclosure;

FIG. 28 are photographs of immunohistochemical (IHC) staining and H & E staining of a tumor treated by BsAb of Example 1.2, murine OKT3 T cells, or murine OKT3 T cells modified with anti-EGFR Fab/anti-CD3 scFv of Example 1.2 in accordance with one embodiment of the present disclosure;

FIG. 29A illustrates flow cytometry analysis on the T cells differentiated by murine OKT3, and anti-PSMA Fab/anti-CD3 scFv, or anti-PSMA scFv/anti-CD3 scFv in accordance with one embodiment of the present disclosure;

FIG. 29B illustrates the proliferation of T cells respectively differentiated by murine OKT3, and anti-CD3 Fab/anti-PSMA scFv, or anti-PSMA scFv/anti-CD3 scFv in accordance with one embodiment of the present disclosure;

FIG. 30 illustrates the flow cytometry analysis on two types antibody fragments carried by OKT3 T cells or by T cells armed with anti-CD3 Fab/anti-PSMA scFv or anti-PSMA scFv/anti-CD3 scFv in accordance with one embodiment of the present disclosure;

FIG. 31 illustrates the respective cytotoxicities of murine OKT3 T cells and T cells of example 2 on LNCaP cells in accordance with one embodiment of the present disclosure;

FIG. 32 illustrates the induction of CD4⁺FoxP3⁺ regulator T cells by anti-CD3 Fab/anti-PSMA scFv BsAb and anti-PSMA scFv/anti-CD3 scFv in accordance with one embodiment of the present disclosure;

FIG. 33 is the flow cytometry analysis of the BsAb on the surfaces of the proliferated T cells in accordance with one embodiment of the present disclosure;

FIG. 34 depicts the cytotoxicity analysis of anti-EGFR/anti-CD3 BsAb-armed T cells and OKT3-armed T cells on colorectal cancer cell line HT-29 in accordance with one embodiment of the present disclosure;

FIG. 35 are bar graphs depicting respective levels of cytokines secreted by anti-EGFR/anti-CD3 BsAb-armed T cells or OKT3-armed T cells at various effector:target ratios (E/T ratios) in accordance with one embodiment of the present disclosure;

FIG. 36 illustrates the quantified fluorescent intensity of anti-EGFR/anti-CD3 BsAb-armed T cells or OKT3-armed T cells in tumors of the test animal in accordance with one embodiment of the present disclosure;

FIG. 37 illustrates the distribution pattern of anti-EGFR/anti-CD3 BsAb-armed T cells or OKT3-armed T cells in tumors and various organs of the test animal in accordance with one embodiment of the present disclosure; and

FIG. 38 is a line graph depicting changes in the size of a tumor treated by specified treatment in accordance with one embodiment of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

I. Definition

The term “antibody” is used in the broadest sense and specifically covers monoclonal antibodies, polyclonal antibodies, multi-specific antibodies (e.g., bi-specific antibodies), and antibody fragments so long as they exhibit the desired biological activity, that is, to specifically bind to an antigen when it preferentially recognizes its target antigen in a complex mixture of proteins and/or other molecules.

The term “monoclonal antibody” as used herein refers to an antibody obtained from a population of substantially homogeneous antibodies, and is not to be constructed as requiring production of the antibody by any particular method. In contrast to polyclonal antibodies which typically include different antibodies directed to different epitopes, each monoclonal antibody is directed against a single determinant (i.e., epitope) on the antigen. The monoclonal antibodies of the present disclosure may be made by hybridoma method or by recombinant DNA methods. The monoclonal antibodies herein specifically include “chimeric” or “recombinant” antibodies, in which a portion of the heavy and/or light chain is identical with or homologous to corresponding sequences in antibodies derived from a particular species or belonging to a antibody class or subclass, while the remainder of the chain identical with or homologous to corresponding sequences in antibodies derived from another species or belonging to another antibody class or subclass, as well as fragments of such antibodies, as long as they exhibit the desired biological activity.

“Humanized” forms of non-human (e.g., murine) antibodies are chimeric antibodies which contain minimal sequence derived from non-human immunoglobulin. Humanized antibodies are human immunoglobulins in which hypervariable region residues are replaced by hypervariable region residues from a non-human species such as mouse, rat, rabbit, or non-human primate having the desired specificity or affinity. In some instances, Fv framework region (FR) residues of the human immunoglobulin are replaced by corresponding non-human residues. In general, the humanized antibody will comprise substantially all of at least one, and typically two, variable domains, in which all or substantially all of the FR regions are those of a human immunoglobulin sequence. The humanized antibody may optionally comprise at least a portion of an immunoglobulin constant region (Fc), typically that of a human immunoglobulin.

The term “bi-specific antibody (BsAb)” refers to an antibody having specificities for at least two different antigens. In preferred embodiments, the BsAb of the present disclosure has two antigen-binding sites, in which one is directed against a tumor antigen (e.g., EGFR, PD-L1, HER2, PSMA and etc), while the other is directed against a T cell co-receptor (e.g., CD3).

The term “linker” and “peptide linker” are interchangeably used in the present disclosure and refers to a peptide having natural or synthetic amino acid residues for connecting two polypeptides. For example, the peptide linker may be used to connect the VH and the VL to form the single chain variable fragment (e.g., scFv); or to connect the scFv to the full length antibody to form a BsAb of the present disclosure. Preferably, the linker is a peptide having at least 5 amino acid residues in length, such as 5 to 100 amino acid residues in length, more preferably 10 to 30 amino acid residues in length. The linker within scFv is a peptide of at least 5 amino acid residues in length, preferably 15 to 20 amino acid residues in length. Preferably, the linker comprises a sequence of (GₙS)ₘ, with G=glycine, S=serine, and n and m are independently a number between 1 to 4. In one example, the linker comprises a sequence of (G₂S)₄. In another example, the linker comprises a sequence or (G₄S)₃.

The terms “cancer” and “tumor” are used alternatively in the present disclosure and preferably refer to the physiological condition in mammals and especially in humans that is typically characterized by un-regulated cell growth. Cancers in this respect include metastases cancers, and/or drug-resistant cancers. Cancers, preferably those exhibit increased expression levels of αvβ3, α5β1, carcinoembryonic antigen (CEA), cytotoxic T-lymphocyte-associated antigen 4 (CTLA4), CD3, CD19, CD20, CD30, CD50, CIAX, cMuc1, ED-B, epidermal growth factor receptor (EGFR), epithelia cell adhesion molecules (EpCAM), erythropoietin-producing hepatocellular A3 (EPHA3), familial adenomatous polyposis (FAP), gpA33, Globo-H, human epidermal growth factor receptor 2 (HER2), HER3, insulin like growth factor 1 receptor (IGF1R), OC183B2, platelet-derived growth factor receptor (PDGFR), programed cell death ligand 1 (PD-L1), prostate specific membrane antigen (PSMA), Le^{y}, MET, tumor-associated glycoprotein 72 (TAG72), Tenascin, vascular endothelial growth factor receptor (VEGFR), VEGFR-2, and/or VEGFR-3. Accordingly, cancers or tumors treatable by the present disclosure are breast, lung, colon, colorectal, spleen, kidney, liver, bladder, head and neck, ovary, prostate, brain, pancreas, skin, bone, blood, thymus, uterus, testicles, cervix, and neuron. More specifically, the cancer is selected from the group consisting of bladder cancer, biliary cancer, bone cancer, brain tumor, breast cancer, cervical cancer, colorectal cancer, colon cancer, esophageal cancer, epidermal carcinoma, gastric cancer, gastrointestinal stromal tumor (GIST), glioma, hematopoietic tumors of lymphoid lineage, hepatic cancer, non-Hodgkin's lymphoma, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer. In one example, the caner is a malignant pancreatic cancer. In another example, the cancer is triple-negative breast cancer (TNBC).

The term “an effective amount” as used herein refers to an amount effective, at dosages, and for periods of time necessary, to achieve the desired therapeutically desired result with respect to the treatment of cancers.

The term “administered,” “administering” or “administration” are used interchangeably herein to refer means either directly administering a BsAb of the present disclosure, T cells differentiated and proliferated by the BsAb of the present disclosure, or a combination thereof.

The term “subject” or “patient” refers to an animal including the human species that is treatable with the compositions and/or methods of the present disclosure. The term “subject” or “patient” intended to refer to both the male and female gender unless one gender is specifically indicated. Accordingly, the term “subject” or “patient” comprises any mammal which may benefit from treatment of cancer. Examples of a “subject” or “patient” include, but are not limited to, a human, rat, mouse, guinea pig, monkey, pig, goat, cow, horse, dog, cat, bird and fowl. In an exemplary embodiment, the patient is a human.

The term “identical” or “percent identity” as used herein refers to two or more sequences or subsequences that are the same or have a specified percentage of amino acid residues that are the same, when compared and aligned for maximum correspondence. To determine the percent identity, the sequences are aligned for optimal comparison purposes (e.g., gaps can be introduced in the sequence of a first amino acid sequence for optimal alignment with a second amino acid sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In certain embodiments, the two sequences are the same length.

The singular forms “a,” “and,” and “the” are used herein to include plural referents unless the context clearly dictates otherwise.

II. Description of the Invention

It is the first aspect of the present disclosure to provide antigen-specific T cells, particularly T cells that are activated (or produced) by recombinant bi-specific antibodies (BsAbs) of the present disclosure. According to embodiments of the present disclosure, the antigen-specific T cells bear specific anti-tumor antigen fragment on the surfaces.

Accordingly, such antigen-specific T cells would bind more strongly to tumor cells including malignant tumor cells and triple-negative breast cancer (TNBC) cells, thereby may suppress the growth and metastasis of the tumor cells.

1. The BsAbs of the Present Disclosure

Antibodies belong to the immunoglobulin class of proteins that includes IgG, IgA, IgE, IgM, and IgD. The most abundant immunoglobulin found in serum is IgG, which has four chains, two light chains and two heavy chains; each light chain has two domains and each heavy chain has four domains. The antigen-binding site is located in the fragment antigen binding (Fab) region that contains a variable light (VL) and variable heavy (VH) chain domains as well as a constant light (CL) and constant heavy (CH1) domains. The CH2 and CH3 domain region of the heavy chain is called fragment crystallizable (Fc) region. A full length antibody heavy chain is therefore a polypeptide consisting of, from N-terminus to C-terminus, a VH, a CH1, a hinge region (HR), a CH2, and a CH3; abbreviated as VH-CH1-HR-CH2-CH3. A full length antibody light chain is a polypeptide consisting in N-terminus to C-terminus direction of a VL and a CL, abbreviated as VL-CL, in which the CL can be κ (kappa) or λ (lambda). The IgG is regarded as a heterotetramer having two heavy chains that are held together by disulfide bonds (—S—S—) between the CL domain and the CH1 domain and between the hinge regions of the two heavy chains.

As stated above in the “definition” section, the BsAbs refer to Abs having specificities for different antigens; hence, BsAbs of the present disclosure is a recombinant Ab engineered to contain sequences capable of binding to two different antigens, which are a tumor antigen and a CD3 antigen (also known as “CD3 complex”, a T cell co-receptor involved in the activation of both cytotoxic T cells (CD8⁺ T cells) and T helper cells (CD4⁺ cells)). Accordingly, each BsAbs of the present disclosure contains an anti-tumor antigen sequence (i.e., an anti-tumor antigen fragment) and an anti-CD3 sequence (i.e., an anti-CD3 fragment). Various recombinant BsAbs have been developed in the present disclosure, and are used to induce the activation (differentiation and/or proliferation) of antigen specific T cells from PBMCs, in which the antigen specific T cells bear on their surfaces the anti-tumor antigen fragment of BsAbs.

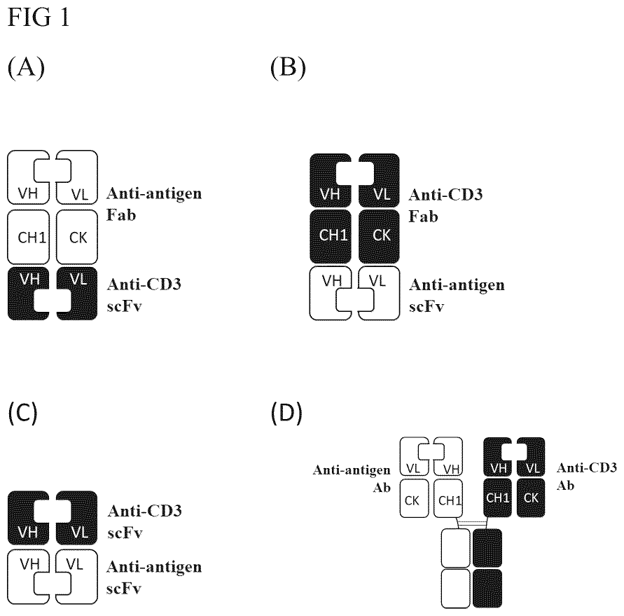

Reference is made to FIG. 1, which illustrates the schematic structures of BsAbs suitable for use in the present disclosure.

In some embodiments, the BsAb of the present disclosure is monomeric, divalent bi-specific antibody, in which a VH-CH1 domain and a light chain VL-Ck directed to a tumor antigen (i.e., anti-tumor antigen Fab) is fused to an anti-CD3 scFv (scFv) consisting of a heavy chain domain (VH) and a light chain variable domain (VL), abbreviated as VH-VL (FIG. 1A). In other embodiments, the monomeric, divalent BsAb comprises in its structure, an anti-CD3 Fab consisting of a VH-CH1 domain and a light chain VL-Ck directed to CD3, which is fused to an scFv directed to a tumor antigen (FIG. 1B) via a peptide linker. In further embodiments, the monomeric, divalent BsAb of the present disclosure is composed of, an anti-tumor antigen scFv and an anti-CD3 scFv, in which both scFvs are connected via a peptide linker (FIG. 1C). In still further embodiments, the BsAb of the present disclosure has a “knob into hole” structure, in which a knob in the CH3 domain of the first heavy chain is created by replacing several amino acid side chains with alternative ones, and a hole in the juxtaposed position at the CH3 domain of the second heavy chain is created by replacing appropriate amino acid side chains with alternative ones. A schematically presentation of the “knob into hole” BsAb structure is depicted in FIG. 1D. The knob-in-hole technique is well known to those skilled in the art, and can be readily applied in forming the BsAbs of the present disclosure.

DNAs encoding the present BsAbs are derived from known antibodies, their genes are cloned and fused to create DNA constructs of desired humanized BsAbs. Detailed production method is set forth in the Examples.

Accordingly, recombinant BsAb having binding sites to the CD3 of a T cell, and a tumor antigen selected from the group consisting of PSMA, EGFR, PD-L1, CEA, FAP, EpCAM, HER2, and VCAM-1, are created.

According to some embodiments, anti-PSMA/anti-CD3 BsAbs are produced. In on example, the anti-PSMA/anti-CD3 BsAb comprises a PSMA binding site comprising aVH-CH1 domain of SEQ ID NO: 65 and a VL-Ck domain of SEQ ID NO: 64; and a CD3 binding site comprising an anti-CD3 scFv of SEQ ID NO: 66. In another example, the anti-PSMA/anti-CD3 BsAb comprises a PSMA binding site comprising an anti-PSMA scFv of SEQ ID NO: 69; and a CD3 binding site comprising aVH-CH1 domain of SEQ ID NO: 68 and a VL-Ck domain of SEQ ID NO: 67. In a further example, the anti-PSMA/anti-CD3 BsAb comprises a PSMA binding site comprising an anti-PSMA scFv of SEQ ID NO: 70; and a CD3 binding site comprising an anti-CD3 scFv of SEQ ID NO: 66. In still a further example, the anti-PSMA/anti-CD3 BsAb has a “knob into hole” structure, in which the CD3 binding site comprises a VL-Ck knob domain of SEQ ID NO: 71 and a VH-CH1 knob domain of SEQ ID NO: 72; and the PSMA binding site comprises a VL-Ck hole domain of SEQ ID NO: 73 and a VH1-CH1 hole domain of SEQ ID NO: 74.

According to some embodiments, anti-EGFR/anti-CD3 BsAbs are produced. In on example, the anti-EGFR/anti-CD3 BsAb comprises an EGFR binding site comprising aVH-CH1 domain of SEQ ID NO: 75 and a VL-Ck domain of SEQ ID NO: 76; and a CD3 binding site comprising an anti-CD3 scFv of SEQ ID NO: 66. In another example, the anti-EGFR/anti-CD3 BsAb comprises an EGFR binding site comprising an anti-EGFR scFv of SEQ ID NO: 77; and a CD3 binding site comprising aVH-CH1 domain of SEQ ID NO: 68 and a VL-Ck domain of SEQ ID NO: 67. In a further example, the anti-EGFR/anti-CD3 BsAb comprises an EGFR binding site comprising an anti-EGFR scFv of SEQ ID NO: 78; and a CD3 binding site comprising an anti-CD3 scFv of SEQ ID NO: 79. In still a further example, the anti-EGFR/anti-CD3 BsAb has a “knob into hole” structure, in which the CD3 binding site comprises a VL-Ck knob domain of SEQ ID NO: 71 and a VH-CH1 knob domain of SEQ ID NO: 72; and the EGFR binding site comprises a VL-Ck hole domain of SEQ ID NO: 80 and a VH1-CH1 hole domain of SEQ ID NO: 81.

According to some embodiments, anti-PD-L1/anti-CD3 BsAbs are produced. In on example, the anti-PD-L1/anti-CD3 BsAb comprises a PD-L1 binding site comprising aVH-CH1 domain of SEQ ID NO: 83 and a VL-Ck domain of SEQ ID NO: 82; and a CD3 binding site comprising an anti-CD3 scFv of SEQ ID NO: 66. In another example, the anti-PD-L1/anti-CD3 BsAb comprises a PD-L1 binding site comprising an anti-PD-L1 scFv of SEQ ID NO: 84; and a CD3 binding site comprising aVH-CH1 domain of SEQ ID NO: 68 and a VL-Ck domain of SEQ ID NO: 67. In a further example, the anti-PD-L1/anti-CD3 BsAb comprises a PD-L1 binding site comprising an anti-PD-L1 scFv of SEQ ID NO: 85; and a CD3 binding site comprising an anti-CD3 scFv of SEQ ID NO: 79. In still a further example, the anti-PD-L1/anti-CD3 BsAb has a “knob into hole” structure, in which the CD3 binding site comprises a VL-Ck knob domain of SEQ ID NO: 71 and a VH-CH1 knob domain of SEQ ID NO: 72; and the PD-L1 binding site comprises a VL-Ck hole domain of SEQ ID NO: 86 and a VH1-CH1 hole domain of SEQ ID NO: 87.

According to some embodiments, anti-HER2/anti-CD3 BsAbs are produced. In on example, the anti-HER2/anti-CD3 BsAb comprises a HER2 binding site comprising aVH-CH1 domain of SEQ ID NO: 89 and a VL-Ck domain of SEQ ID NO: 88; and a CD3 binding site comprising an anti-CD3 scFv of SEQ ID NO: 66. In another example, the anti-HER2/anti-CD3 BsAb comprises a HER2 binding site comprising an anti-HER2 scFv of SEQ ID NO: 90; and a CD3 binding site comprising aVH-CH1 domain of SEQ ID NO: 68 and a VL-Ck domain of SEQ ID NO: 67. In a further example, the anti-HER2/anti-CD3 BsAb comprises a HER2 binding site comprising an anti-HER2 scFv of SEQ ID NO: 91; and a CD3 binding site comprising an anti-CD3 scFv of SEQ ID NO: 79. In still a further example, the anti-HER2/anti-CD3 BsAb has a “knob into hole” structure, in which the CD3 binding site comprises a VL-Ck knob domain of SEQ ID NO: 71 and a VH-CH1 knob domain of SEQ ID NO: 72; and the HER2 binding site comprises a VL-Ck hole domain of SEQ ID NO: 92 and a VH1-CH1 hole domain of SEQ ID NO: 93.

According to some embodiments, anti-FAP/anti-CD3 BsAbs are produced. In on example, the anti-FAP/anti-CD3 BsAb comprises an FAP binding site comprising aVH-CH1 domain of SEQ ID NO: 95 and a VL-Ck domain of SEQ ID NO: 94; and a CD3 binding site comprising an anti-CD3 scFv of SEQ ID NO: 66. In another example, the anti-FAP/anti-CD3 BsAb comprises an FAP binding site comprising an anti-FAP scFv of SEQ ID NO: 96; and a CD3 binding site comprising aVH-CH1 domain of SEQ ID NO: 68 and a VL-Ck domain of SEQ ID NO: 67. In a further example, the anti-FAP/anti-CD3 BsAb comprises an FAP binding site comprising an anti-FAP scFv of SEQ ID NO: 97; and a CD3 binding site comprising an anti-CD3 scFv of SEQ ID NO: 79. In still a further example, the anti-FAP/anti-CD3 BsAb has a “knob into hole” structure, in which the CD3 binding site comprises a VL-Ck knob domain of SEQ ID NO: 71 and a VH-CH1 knob domain of SEQ ID NO: 72; and the FAP binding site comprises a VL-Ck hole domain of SEQ ID NO: 98 and a VH1-CH1 hole domain of SEQ ID NO: 99.

According to some embodiments, anti-EpCAM MOC31/anti-CD3 BsAbs are produced. In on example, the anti-EpCAM MOC31/anti-CD3 BsAb comprises an EpCAM MOC31 binding site comprising a VH-CH1 domain of SEQ ID NO: 101 and a VL-Ck domain of SEQ ID NO: 100; and a CD3 binding site comprising an anti-CD3 scFv of SEQ ID NO: 66. In another example, the anti-EpCAM MOC31/anti-CD3 BsAb comprises an EpCAM MOC31 binding site comprising an anti-EpCAM MOC31 scFv of SEQ ID NO: 102; and a CD3 binding site comprising aVH-CH1 domain of SEQ ID NO: 68 and a VL-Ck domain of SEQ ID NO: 67. In a further example, the anti-EpCAM MOC31/anti-CD3 BsAb comprises an EpCAM MOC31 binding site comprising an anti-EpCAM MOC31 scFv of SEQ ID NO: 103; and a CD3 binding site comprising an anti-CD3 scFv of SEQ ID NO: 79. In still a further example, the anti-EpCAM MOC31/anti-CD3 BsAb has a “knob into hole” structure, in which the CD3 binding site comprises a VL-Ck knob domain of SEQ ID NO: 71 and a VH-CH1 knob domain of SEQ ID NO: 72; and the EpCAM MOC31 binding site comprises a VL-Ck hole domain of SEQ ID NO: 104 and a VH1-CH1 hole domain of SEQ ID NO: 105.

According to some embodiments, anti-EpCAM MT201/anti-CD3 BsAbs are produced. In on example, the anti-EpCAM MT201/anti-CD3 BsAb comprises an EpCAM MT201 binding site comprising a VH-CH1 domain of SEQ ID NO: 107 and a VL-Ck domain of SEQ ID NO: 106; and a CD3 binding site comprising an anti-CD3 scFv of SEQ ID NO: 66. In another example, the anti-EpCAM MT201/anti-CD3 BsAb comprises an EpCAM MT201 binding site comprising an anti-EpCAM MT201 scFv of SEQ ID NO: 108; and a CD3 binding site comprising aVH-CH1 domain of SEQ ID NO: 68 and a VL-Ck domain of SEQ ID NO: 67. In a further example, the anti-EpCAM MT201/anti-CD3 BsAb comprises an EpCAM MT201 binding site comprising an anti-EpCAM MT201 scFv of SEQ ID NO: 109; and a CD3 binding site comprising an anti-CD3 scFv of SEQ ID NO: 79. In still a further example, the anti-EpCAM MT201/anti-CD3 BsAb has a “knob into hole” structure, in which the CD3 binding site comprises a VL-Ck knob domain of SEQ ID NO: 71 and a VH-CH1 knob domain of SEQ ID NO: 72; and the EpCAM MT201 binding site comprises a VL-Ck hole domain of SEQ ID NO: 110 and a VH1-CH1 hole domain of SEQ ID NO: 111.

According to some embodiments, anti-CEA/anti-CD3 BsAbs are produced. In on example, the anti-CEA/anti-CD3 BsAb comprises a CEA binding site comprising a VH-CH1 domain of SEQ ID NO: 113 and a VL-Ck domain of SEQ ID NO: 112; and a CD3 binding site comprising an anti-CD3 scFv of SEQ ID NO: 66. In another example, the anti-CEA/anti-CD3 BsAb comprises a CEA binding site comprising an anti-CEA scFv of SEQ ID NO: 114; and a CD3 binding site comprising aVH-CH1 domain of SEQ ID NO: 68 and a VL-Ck domain of SEQ ID NO: 67. In a further example, the anti-CEA/anti-CD3 BsAb comprises a CEA binding site comprising an anti-CEA scFv of SEQ ID NO: 115; and a CD3 binding site comprising an anti-CD3 scFv of SEQ ID NO: 79. In still a further example, the anti-CEA/anti-CD3 BsAb has a “knob into hole” structure, in which the CD3 binding site comprises a VL-Ck knob domain of SEQ ID NO: 71 and a VH-CH1 knob domain of SEQ ID NO: 72; and the CEA binding site comprises a VL-Ck hole domain of SEQ ID NO: 116 and a VH1-CH1 hole domain of SEQ ID NO: 117.

According to some embodiments, anti-VCAM1/anti-CD3 BsAbs are produced. In on example, the anti-VCAM1/anti-CD3 BsAb comprises a VCAM1 binding site comprising a VH-CH1 domain of SEQ ID NO: 119 and a VL-Ck domain of SEQ ID NO: 118; and a CD3 binding site comprising an anti-CD3 scFv of SEQ ID NO: 66. In another example, the anti-VCAM1/anti-CD3 BsAb comprises a VCAM1 binding site comprising an anti-VCAM1 scFv of SEQ ID NO: 120; and a CD3 binding site comprising aVH-CH1 domain of SEQ ID NO: 68 and a VL-Ck domain of SEQ ID NO: 67. In a further example, the anti-VCAM1/anti-CD3 BsAb comprises a VCAM1 binding site comprising an anti-VCAM1 scFv of SEQ ID NO: 121; and a CD3 binding site comprising an anti-CD3 scFv of SEQ ID NO: 79. In still a further example, the anti-VCAM1/anti-CD3 BsAb has a “knob into hole” structure, in which the CD3 binding site comprises a VL-Ck knob domain of SEQ ID NO: 71 and a VH-CH1 knob domain of SEQ ID NO: 72; and the VCAM1 binding site comprises a VL-Ck hole domain of SEQ ID NO: 122 and a VH1-CH1 hole domain of SEQ ID NO: 123.

According to further embodiments of the present disclosure, humanized OKT3 Anti-CD3 VH and CL are produced, these sequences (see Tables 39 to 42) may fused with relevant anti-tumor antigen sequences described in the present disclosure to produce desired BsAbs.

2. Antigen Specific T Cells Activated by the Present BsAbs

The BsAbs of the present disclosure are used to produce antigen specific T cells from PBMCs. Accordingly, one aspect of the present disclosure is directed to the production of antigen specific T cells, which respectively have the BsAbs bound on their surfaces, i.e., being armed with the BsAbs, and thus exhibit binding affinity to the corresponding tumor antigen via the anti-tumor antigen fragment of the BsAbs.

Accordingly, the present disclosure encompasses a method of inducing differentiation and/or proliferation of T cells from PBMCs. The method comprises, culturing PBMCs harvested from a subject with bi-specific antibodies (BsAbs) of the present disclosure in a medium, so as to induce the activation (proliferation and/or differentiation) of the T cells (e.g., CD3⁺CD8⁺ T cells) from the PBMCs, wherein, each BsAbs comprises a tumor antigen binding site that recognizes and binds to a corresponding tumor antigen, and a CD3 binding site that recognizes and binds to the CD3 molecule of T cells. In this case, the BsAb is useful for binding to and activating the circulating T cells in the PBMCs to proliferate and/or differentiate into specific T cell subsets (e.g., CD3⁺, CD4⁺ or CD8⁺ T cells) via its CD3 binding site, and allowing the activated T cells to specifically target to cancerous cells expressing the tumor antigen via its tumor antigen binding site. According to the embodiments of the present disclosure, the BsAbs are not murine OKT3 antibodies.

PBMCs may be isolated from fresh blood of a subject using any methods known to a skilled artisan, such as by density centrifugation (Ficoll-Paque™), as different components of the blood have different densities and can be separated accordingly.

To achieve differentiation and proliferation of T cells, the isolated PBMCs are cultivated with any of BsAbs of the present disclosure in a normal culture medium for a sufficient period of time, for example, at least 7 days, such as 7, 8, 9, 10, 11, 12, 13, and 14 days; and preferably for at least 14 days. In some embodiments, the number of CD3⁺ T cells multiplies after cultivation for 7 days. In other embodiments, cultivation is continued for 14 days, and the number of CD3⁺ T cells increases for 3 folds. In addition or optionally, the culture medium may include a cytokine, such as IL-2, IL-7 and a combination thereof.

As described above, each T cells thus produced comprises a tumor antigen binding site on its surface that recognizes and binds to a corresponding tumor antigen. In some examples, each T cells thus produced comprises an anti-PSMA fragment on its surface, and accordingly exhibits binding affinity to PSMA-positive cell. In other examples, each T cells thus produced comprises an anti-EGFR fragment on its surface, and accordingly exhibits binding affinity to EGFR-positive cell. In further examples, each T cells thus produced comprises anti-PD-L1 fragment on its surface, and accordingly exhibits binding affinity to PD-L1-positive cell.

According to certain embodiments of the present disclosure, cytotoxic T cells (CD3⁺CD8⁺ T cells) are produced after cultivating PBMCs with the BsAbs of the present disclosure for at least 7 days (for example, 7 days or 14 days). In these embodiments, the culture medium comprises IL-2 only, without any additional cytokines (e.g., TGF-β).

According to further embodiments of the present disclosure, periphery derived regulatory T cells are produced after cultivating PBMCs with the BsAbs of the present disclosure for at least 7 days, in which cell marker FoxP3 appeared on the surface of the T cells. In such case, the culture medium further comprises anti-CD28 antibodies, in addition to the cytokine, IL-2.

According to embodiments of the present disclosure, antigen specific T cells (including the periphery derived regulatory T cells) of the present disclosure not only exhibit stronger binding affinity toward tumor cells, but also produce higher levels of cytokines, including, but are not limited to, IL-2, IFN-γ, TNF-α, granzyme B and perforin. Accordingly, these T cells exhibit much higher level of cytotoxicity toward tumor cells, including malignant cancer cells and TNBC cells, as compared with T cells activated by murine OKT3 Abs.

3. Methods of Treating Cancers

3.1 Treating Cancer by Use of Antigen Specific T Cells

Accordingly, it is a further aspect of the present disclosure to provide a method of treating cancers. The method takes advantages of antigen specific T cells described in Section 2, in which an effective amount of the T cells per se, or T cells that are further modified with BsAbs, is administered to a subject afflicted with a cancer, so as to suppress or inhibit the growth of the cancer cells.

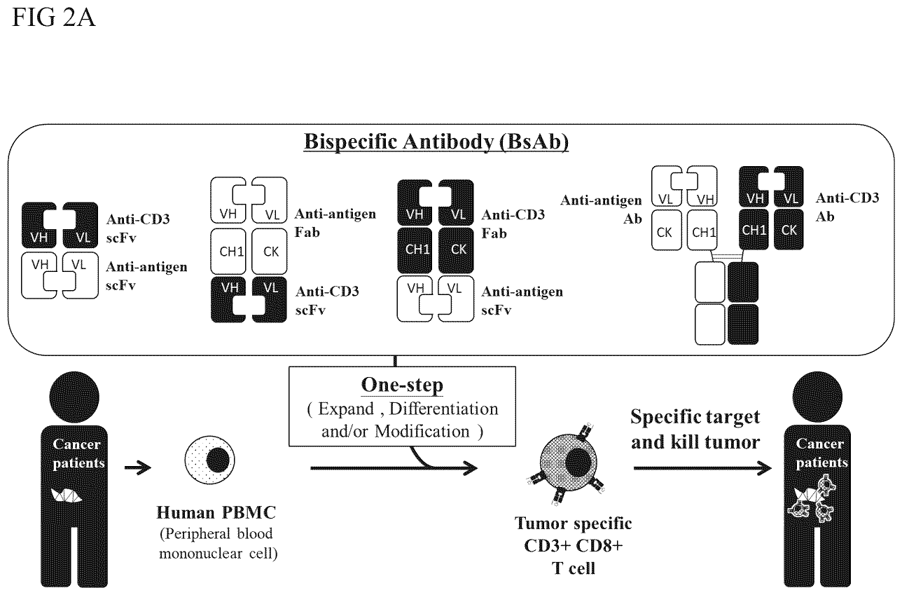

Reference is made to FIG. 2A, which is a schematic presentation of the present method, in which PBMCs are first isolated from fresh blood of a subject, then subjected to one-step expanding and differentiation treatment to arm the differentiated T cells with the BsAbs. Specifically, the one-step expanding and differentiation treatment incudes cultivating PBMCs with the BsAbs of the present disclosure in a culture medium for at least 7 days, more preferably, for at least 14 days, until sufficient number of desired T cells are produced. In addition or optionally, the antigen specific T cells thus produced may be further modified with the BsAbs, in which case, the present antigen specific T cells are further incubated with the BsAbs of the present disclosure, such step is termed “one-step expanding, differentiation and modification” treatment depicted in FIG. 2A.

In some embodiments, an effective amount of the antigen specific T cells (i.e., without further modification with the BsAbs) are administered directly to the subject for treating cancer. In other embodiments, an effective amount of the antigen specific T cells further modified with the BsAbs are administered to the subject for treating cancer.

The amount of T cells administered to the subject is from about 1×10⁴ to 1×10⁷ cells/Kg body weight of the subject. In certain embodiments, the amount of T cells is administered to the subject from about 1×10⁵ to 1×10⁶ cells/Kg body weight of the subject. The dose can be administered in a single dose, or alternatively in more than one smaller doses.

3.2 Treating Cancer by Use of Murine OKT3 T Cells Modified with the Present BsAbs

In some embodiments, instead of using the antigen specific T cells described above in Section 2, murine OKT3 T cells (i.e., T cells derived by activating PBMCs via murine OKT3 Abs) further modified with the BsAbs of the present disclosure are used for the treatment of a cancer. Reference is made to FIG. 2B, which is similar to FIG. 2A, except murine OKT3 T cells are modified with the BsAbs of the present disclosure and administered to the subject. Similar to FIG. 2A, the modification is achieved by cultivating murine OKT3 T cells with the BsAbs of the present disclosure, so as to arm murine OKT3 T cells with the anti-tumor antigen and the anti-CD3 of the BsAbs.

According to embodiments of the present disclosure, at least 500 ng, such as 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, and 3,000 ng of BsAbs of the present disclosure is sufficient to modify murine OKT3 T cells (e.g., 3×10⁵ cells) so that the desired anti-tumor antigen of the BsAbs of the present disclosure are expressed on their surfaces. In some examples, murine OKT3 T cells are further modified with anti-PSMA/anti-CD3 BsAbs of the present disclosure. In further examples, murine OKT3 T cells modified with anti-PSMA/anti-CD3 BsAbs of the present disclosure produce much higher level of cytokines, including, but are not limited to, IL-2, IFN-γ, TNF-α, Granzyme B and Perforin, as compared to unmodified murine OKT3 T cells. Accordingly, these modified murine OKT3 T cells exhibit much higher level of cytotoxicity toward tumor cells, including malignant cancer cells and TNBC cells, as compared with unmodified murine OKT3 T cells.

The BsAbs, antigen specific T cells, as well as the modified murine OKT3 T cells of the present disclosure may be administered to a mammal, preferably human, by any route that may effectively transport the BsAbs or T cells to desired site of action, such as oral, nasal, pulmonary, transdermal, such as passive or iontophoretic delivery, or parenteral, e.g., rectal, depot, subcutaneous, intravenous, intramuscular, intranasal, intra-cerebella, ophthalmic solution or an ointment. It will be appreciated that the dosage of the present disclosure will vary from patient to patient not only for the cancer therapeutic agent selected, the route of administration, and the ability of the BsAb, the T cells, and/or a combination thereof, to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the improved therapeutic response. A therapeutically effective amount is also one in which any toxic or detrimental effects of the cancer therapeutic agent are outweighed by the therapeutically beneficial effects. Preferably, the BsAb, T cells, or a combination thereof, are administered at a dosage and for a time such that the growth of the cancer cells are suppressed.

The present invention will now be described more specifically with reference to the following embodiments, which are provided for the purpose of demonstration rather than limitation.

### EXAMPLES

Materials and Methods.

Cells and Animals

Human T lymphocyte cell line Jurkat T cells (CD3⁺), human prostate adenocarcinoma cell line LNCaP (PSMA⁺/PD-L1⁺), human colon adenocarcinoma cell line HT-29 (EGFR⁺), malignant pancreatic cell line MIA PaCa-2 (PD-L1⁺), triple negative breast cancer (TNBC) cell line MDA-MB-231 (EGFR⁺/PD-L1⁺) and Expi293™ cells were used in the present disclosure.

In general, HT-29, MIA PaCa-2 and MDA-MB-231 cells were cultured in Dulbecco's modified Eagle's medium (Sigma, St Louis, MO, USA) supplemented with 10% fetal calf serum (HyClone, Logan, UT), 100 U/mL penicillin and 100 μg/mL streptomycin at 37° C. in an atmosphere of 5% CO₂ in air. Jurkat T and LNCaP cells were grown in RPMI-1640 containing the same supplements. Expi293™ cells were maintained in Expi293™ Expression medium at 37° C. in an atmosphere of 5% CO₂ in air.

SCID mice (6-8 weeks old) were obtained from the National Laboratory Animal Center, Taipei, Taiwan. All animal experiments were performed in accordance with institutional guidelines and approved by the Laboratory Animal Facility and Pathology Core Committee of Taipei Medical University (Taipei, Taiwan).

Production of Recombinant BsAbs

To generate the BsAbs of the present disclosure, nucleic acids encoding the anti-tumor antigen (e.g., anti-PSMA, anti-EGFR, anti-PD-L1 and etc) and anti-CD3 were grafted and fused with other DNA sequence (e.g., signal peptide, IRES, linker and etc) into desired constructs via whole gene synthesis.

The thus produced DNA constructs were then amplified in Expi293™ cells (7.5×10⁷ cell/25.5 mL, with the addition of 30 μg nucleic acids for transfection), which were cultured at 37° C. in an atmosphere of 8% CO₂ in air. BsAbs were purified from the culture media collected after 6 days by Ni-Affinity chromatography, and the concentration was determined by use of Pierce™ BCA Protein Assay kit.

Production of Humanized OKT3 VH and VL

To select human framework sequences for complementarity-determining region (CDR) grafting, we compared the VH and VL sequences of the OKT3 with the National Center for Biotechnology Information database of human immunoglobulin germline sequences in the variable and joining regions using the IgBLAST program. The IGHV1-4603/IGHJ4 was found to be the most homologous to the variable/joining regions of OKT3 VH. The IGKV1-39\hat{ }01/IgKJ4 was found to be the most homologous to the variable and joining regions of OKT3 VL. For construction of the humanized OKT3 VH, the CDRs determined by the rule of Kabat et al. (Sequences of Proteins of Immunological Interest. 5th ed. Bethesda, MD: U.S. Department of Health and Human Services, Public Health Service, National Institutes of Health; 1991:103-511) and 2 residues (Thr 71 and Lys 73) of OKT3 VH were similarly grafted into IGHV1-46\hat{ }03/IGHJ4. For construction of the humanized OKT3 VL, the CDRs determined by the rule of Kabat et al. and 1 residue (Tyr 71) of OKT3 VL were similarly grafted into IGKV1-39\hat{ }01/IgKJ4. The humanized OKT3 VH and VL segments were constructed via whole gene synthesis.

Analysis of the Purfifed BsAbs

BsAbs were electrophoresed in a 10% SDS-PAGE gels under reducing or non-reducing conditions and then stained by Coomassie® Blue.

Differentiation of T Cells

CD8⁺ T cells. Peripheral blood mononuclear cells (PBMCs) isolated from the blood of healthy subjects or naïve CD8 T cells were washed with phosphate buffer solution (PBS) and re-suspended in AIM-V medium at the concentration of 2×10⁶ cells/mL. The suspended cells were then cultivated with BsAbs (1-1,000 ng/mL) and humanized IL-2 (3,000 IU/mL) at 37° C. in an atmosphere of 5% CO₂ in air for designated time. Some of the T cells were collected respectively at 7 and 14 days, and analyzed by flow cytometry.

CD4⁺ T cells. Differentiation of CD4⁺ T cells was relatively the same as that for CD8⁺ T cells, except PBMCs were re-suspended in AIM-V medium at the concentration of 1×10⁶ cells/mL, and cultivated in the presence of BsAbs (1-1,000 ng/mL) and humanized IL-2 (2,000 IU/mL) and IL-7 (20 IU/mL).

Regulatory T cells. Differentiation of regulatory T cells was relatively the same as that for CD8 T cells, except PBMCs or naïve CD4 T cells were suspended in AIM-V medium at the concentration of 1×10⁶ cells/mL, and cultivated in the presence of BsAbs (1-1,000 ng/mL), anti-CD28 antibody (100 ng/mL), humanized IL-2 (2,000 IU/mL) and D-mannose (25 mM).

Flow Cytometer Analysis

Tumor-specific antigen recognition and/or targeting. BsAbs (1 g/mL) were incubated with LNCaP (PSMA+/PD-L1+), HT-29 (EGFR+), MDA-MB-231 (EGFR+/PD-L1+), MIA PaCa-2 (PD-L1+), or Jurkat (CD3+) cells (3×10⁵ cells) at 4° C. for 60 min followed by incubation with mouse anti-histidine antibody (1 μg/mL) and FITC-labeled Goat F(ab)2 anti-mouse immunoglobulin second antibody (1 μg/mL) at 4° C. The fluorescence was measured by FACScalibur™ flow cytometer (Becton Dickinson, Mountain View, CA, USA) then analyzed with Flowjo® (Tree Star Inc., San Carlos, CA, USA).

T cells differentiation. Differentiated T cells (3×10⁵ cells) were mixed with FITC-labeled conjugated anti-human CD3 antibody (1 μg/mL), FITC-labeled conjugated anti-human CD4 antibody (1 μg/mL), FITC-labeled conjugated anti-human CD8 antibody (1 μg/mL), or PE-conjugated anti-human FoxP3 antibody at 4° C. After washing with cold PBS, the fluorescence on the viable cells was measured by FACScalibur™ flow cytometer (Becton Dickinson, Mountain View, CA, USA) then analyzed with Flowjo® (Tree Star Inc., San Carlos, CA, USA).

Amount of BsAbs remained on the surface of T cells. T cells or T cells armed with BsAbs of Example 1 (3×10⁵ cells) were cultivated in a medium containing 20% FCS, and were harvested at 0, 24, 48, and 72 hrs, respectively. After washed with PBS, the harvested T cells were mixed with mouse anti-histidine antibody (1 μg/mL), and FITC-labeled Goat F(ab)2 anti-mouse immunoglobulin second antibody (1 μg/mL) at 4° C. The fluorescence was then measured by FACScalibur™ flow cytometer (Becton Dickinson, Mountain View, CA, USA) then analyzed with Flowjo® (Tree Star Inc., San Carlos, CA, USA).

Cytotoxicity Assay

Tumor cells (10⁴ cells/well) were seeded in 96-well plates and cultivated at 37° C. in an atmosphere of 5% CO₂ in air for 24 hrs. Then, the differentiated T cells (e.g., T cells of Example 2) or OKT3 T cells were added to the tumor cells at E/T ratio of 3:1, 5:1 or 10:1, and continued to cultivate for 16 hrs. The cells were subsequently harvested and the supernatant was analyzed by CytoTox96® Non-Radioactive Cytotoxicity Kit for evaluating the cytotoxicity effect of each differentiated T cells.

ELISA

The level of IL-2, INF-γ, TNF-α, Granzyme B and Perforin secreted from the differentiated T cells were respectively measured by commercial available ELISA kits in accordance with the manufacturer's instruction. Briefly, supernatant of the tumor cells treated with T cells of Example 2 or OKT3 T cells was collected and seeded in 96-well (100 μL/well) and incubated at 36° C. in an incubator for 1.5 hr. After washing with washing buffer, antibodies (100 μL/well) were added, and the mixtures were returned to the incubator and continued incubation at 36° C. for 1 hr. After washing, horse radish peroxidase (HRP) (100 μL/well) was added and continue the incubation for another 30 min. After washing, tetramethylbenzidine (TMB) (100 μL/well) was added, and the mixture was incubated in the dark at 36° C. for 15 min. Then, stop solution (100 μL/well) was added, and the absorbance (405 nm) of wells was measured in a microplate reader.

In Vivo Imaging of SCID Mice Bearing EGFR⁺ Tumors

SCID mice were respectively injected on their back with HT-29 (EGFR+) cells (2×10⁶ cells/100 μL) to induce formation of tumor. The tumor was allowed to grow for 14-17 days until it was about 80-100 mm³ in size, then NIR797-labeled OKT3 T cells further modified with anti-EGFR/anti-CD3 BsAb (10⁷ cells) were injected intravenously into the animals. Pentobarbital anesthetized mice were sequentially imaged with an IVIS spectrum optical imaging system (excitation, 745 nm; emission, 840 nm; Perkin-Elmer, Inc., MA, USA) at 4 and 24 hrs, respectively, after injection. Mice were then sacrificed, and the tumors and organs (including heart, lung, kidney, liver, stomach, muscle, bone, bowl, intestine, pancreas, and blood) were collected and also analyzed by IVIS spectrum optical imaging system.

Treatment of SCID Mice Bearing EGFR⁺ Tumors

SCID mice were respectively injected on their back with HT-29 (EGFR+) cells (2×10⁶ cells/100 μL) to induce formation of tumor. The tumor was allowed to grow for 10-14 days until it was about 30-50 mm³ in size, then NIR797-labeled T cells further modified with anti-EGFR/anti-CD3 BsAb (10⁷ cells/mice) were injected intravenously into the animals. Each mice were weighted and the size of the tumor measured (length×width×height×1/2) twice a week during the treatment period. Mice were then sacrificed, and the tumors were dissected and analyzed by H&E staining, or by immunostaining with anti-CD3 Abs.

Statistical Analysis

Statistical significance of differences between mean values was estimated with JMP 9.0 software (SAS Institute, Inc., Cary, NC) using the nonparametric Mann-Whitney test. P-values in the cytotoxicity assay and in vivo toxicity<0.05 and the P-values in the in vivo treatment<0.01 were considered to be statistically significant. *p<0.05; **p<0.01; ***p<0.001; ns (not significant): >0.05.

### Example 1 Production and Characterization of Humanized Anti-Tumor/Anti-CD3 Antibodies

1.1 Production of Anti-Tumor/Anti-CD3 BsAbs

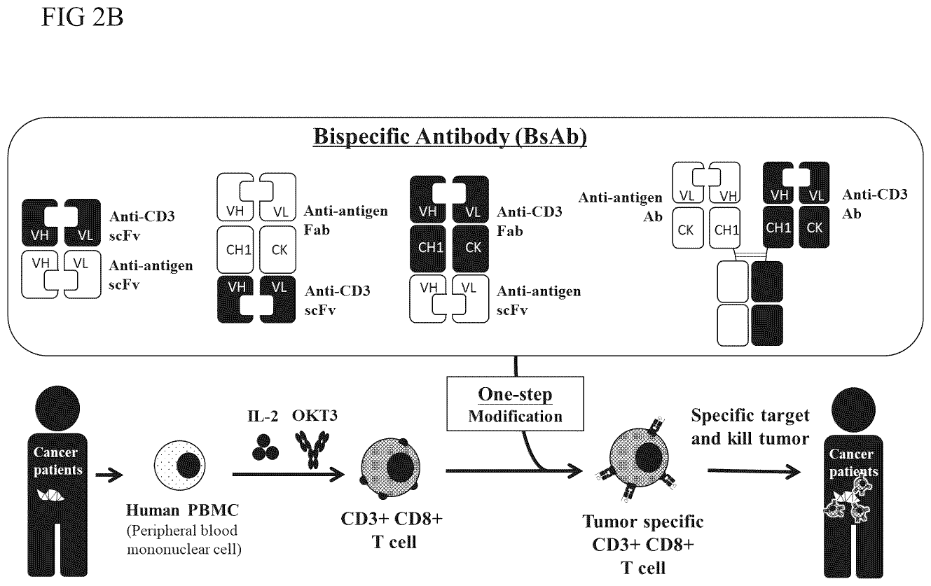

In this example, recombinant humanized bi-specific Abs respectively having the structures as depicted in FIG. 1 were prepared vis use of the DNA constructs illustrated in FIG. 3. For anti-tumor Fab/anti-CD3 scFv BsAb, the construct comprised in sequence, IgG signal peptide (LS), anti-tumor VL-CK (e.g., anti-EGFR VL-CK), internal ribosomal entry site (IRES), anti-tumor VH-CH1 (e.g., anti-EGFR VH-CH1), linker peptide (L), and anti-CD3 VH-VL (FIG. 3, (A)). For anti-CD3 Fab/anti-tumor scFv BsAb, the construct comprised in sequence, LS, anti-CD3 VL-CK, IRES, LS, anti-CD3 VH-CH1, L, and anti-tumor VH-VL (FIG. 3, (B)). For anti-tumor scFv/anti-CD3 scFv BsAb, the construct comprised in sequence, LS, anti-tumor VL-VH, L, and anti-CD3 VH-VL (FIG. 3, (C)). For anti-CD3 knob/anti-tumor hole BsAb, the anti-CD3 knob construct comprised in sequence, LS, anti-CD3 VL-CK, IRES, LS, and anti-CD3 VH-CH1-knob Fc, while the anti-tumor hole comprised in sequence, LS, anti-tumor VL-CK, IRES, LS, and anti-tumor VH-CH1-hole Fc (FIG. 3, (D)).

Accordingly, BsAbs comprised an anti-CD3 and an anti-tumor antigen (i.e., anti-PSMA, anti-EGFR, anti-PD-L1, anti-HER2, and anti-FAP, anti-EpCAM(MOC31), anti-EpCAM(M0201), anti-CEA, and anti-VCAM-1) were prepared. Components and respective nucleic acid and amino acid sequences of the present BsAbs are summarized in Tables 1 to 36. The BsAbs were produced via Expi-293™ expression system, and the yield of each BsAbs was above 100 mg/L, with an assembling accuracy over 95%.

TABLE 1
Components and sequences of anti-
PSMA Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-PSMA VL-Cκ | 2 | 64 |
| IRES | 3 | — |
| Anti-PSMA VH-CH1 | 4 | 65 |
| Anti-CD3 scFv | 5 | 66 |

TABLE 2
Components and sequences of anti-
CD3 Fab/anti-PSMA scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-PSMA scFv | 8 | 69 |

TABLE 3
Components and sequences of anti-
PSMA scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-PSMAscFv | 9 | 70 |
| Anti-CD3 scFv | 5 | 66 |

TABLE 4
Components and sequences of anti-
CD3knob/anti-PSMA hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-PSMA hole-Ck | 12 | 73 |
| Anti-PSMA VH hole CH1-Fc | 13 | 74 |

TABLE 5
Components and sequences of anti-EGFR Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-EGFR VL-Cκ | 14 | 75 |
| IRES | 3 | — |
| Anti-EGFR VH-CH1 | 15 | 76 |
| Anti-CD3 scFv | 5 | 66 |

TABLE 6
Components and sequences of anti-CD3 Fab/anti-EGFR scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-EGFR scFv | 16 | 77 |

TABLE 7
Components and sequences of anti-
EGFR scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-EGFR scFv | 17 | 78 |
| Anti-CD3 scFv | 18 | 79 |

TABLE 8
Components and sequences of anti-
CD3knob/anti-EGFR hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-EGFR hole-Ck | 19 | 80 |
| Anti-EGFR VH hole CH1-Fc | 20 | 81 |

TABLE 9
Components and sequences of anti-
PD-L1 Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-PD-L1 VL-Cκ | 21 | 82 |
| IRES | 3 | — |
| Anti-PD-L1 VH-CH1 | 22 | 83 |
| Anti-CD3 scFv | 5 | 66 |

TABLE 10
Components and sequences of anti-
CD3 Fab/anti-PD-L1 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-PD-L1 scFv | 23 | 84 |

TABLE 11
Components and sequences of anti-
PD-L1 scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-PD-L1 scFv | 24 | 85 |
| Anti-CD3 scFv | 18 | 79 |

TABLE 12
Components and sequences of anti-
CD3 knob/anti-PD-L1 hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-PD-L1 hole-Ck | 25 | 86 |
| Anti-PD-L1 VH hole CH1-Fc | 26 | 87 |

TABLE 13
Components and sequences of anti-HER2 Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-HER2 VL-Cκ | 27 | 88 |
| IRES | 3 | — |
| Anti-HER2 VH-CH1 | 28 | 89 |
| Anti-CD3 scFv | 5 | 66 |

TABLE 14
Components and sequences of anti-CD3 Fab/anti-HER2 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-HER2 scFv | 29 | 90 |

TABLE 15
Components and sequences of anti-
HER2 scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-HER2 scFv | 30 | 91 |
| Anti-CD3 scFv | 18 | 79 |

TABLE 16
Components and sequences of anti-
CD3 knob/anti-HER2 hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-HER2 hole-Ck | 31 | 92 |
| Anti-HER2 VH hole CH1-Fc | 32 | 93 |

TABLE 17
Components and sequences of anti-FAP Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-FAP VL-Cκ | 33 | 94 |
| IRES | 3 | — |
| Anti-FAP VH-CH1 | 34 | 95 |
| Anti-CD3 scFv | 5 | 66 |

TABLE 18
Components and sequences of anti-CD3 Fab/anti-FAP scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-FAP scFv | 35 | 96 |

TABLE 19
Components and sequences of anti-FAP scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-FAP scFv | 36 | 97 |
| Anti-CD3 scFv | 18 | 79 |

TABLE 20
Components and sequences of anti-CD3 knob/anti-FAP hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-FAP hole-Ck | 37 | 98 |
| Anti-FAP VH hole CH1-Fc | 38 | 99 |

TABLE 21
Components and sequences of anti-
EpCAM MOC31 Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-EpCAM MOC31 VL-Cκ | 39 | 100 |
| IRES | 3 | — |
| Anti-EpCAM MOC31 VH-CH1 | 40 | 101 |
| Anti-CD3 scFv | 5 | 66 |

TABLE 22
Components and sequences of anti-
CD3 Fab/anti-EpCAM MOC31 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-EpCAM MOC31 scFv | 41 | 102 |

TABLE 23
Components and sequences of anti-EpCAM
MOC31 scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-EpCAM MOC31 scFv | 42 | 103 |
| Anti-CD3 scFv | 18 | 79 |

TABLE 24
Components and sequences of anti-CD3
knob/anti-EpCAM MOC31 hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-EpCAM MOC31 hole-Ck | 43 | 104 |
| Anti-EpCAM MOC31 VH hole CH1-Fc | 44 | 105 |

TABLE 25
Components and sequences of anti-
EpCAM MT201 Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-EpCAM MT201 VL-Cκ | 45 | 106 |
| IRES | 3 | — |
| Anti-EpCAM MT201 VH-CH1 | 46 | 107 |
| Anti-CD3 scFv | 5 | 66 |

TABLE 26
Components and sequences of anti-
CD3 Fab/anti-EpCAM MT201 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-EpCAM MT201 scFv | 47 | 108 |

TABLE 27
Components and sequences of anti-EpCAM
MT201 scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-EpCAM MT201 scFv | 48 | 109 |
| Anti-CD3 scFv | 18 | 79 |

TABLE 28
Components and sequences of anti-CD3
knob/anti-EpCAM MT201 hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-EpCAM MT201 hole-Ck | 49 | 110 |
| Anti-EpCAM MT201 VH hole CH1-Fc | 50 | 111 |

TABLE 29
Components and sequences of anti-CEA Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CEA VL-Cκ | 51 | 112 |
| IRES | 3 | — |
| Anti-CEA VH-CH1 | 52 | 113 |
| Anti-CD3 scFv | 5 | 66 |

TABLE 30
Components and sequences of anti-CD3 Fab/anti-CEA scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-CEA scFv | 53 | 114 |

TABLE 31
Components and sequences of anti-CEA scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CEA scFv | 54 | 115 |
| Anti-CD3 scFv | 18 | 79 |

TABLE 32
Components and sequences of anti-CD3 knob/anti-CEA hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-CEA hole-Ck | 55 | 116 |
| Anti-CEA VH hole CH1-Fc | 56 | 117 |

TABLE 33
Components and sequences of anti-
VCAM1 Fab/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-VCAM1 VL-Cκ | 57 | 118 |
| IRES | 3 | — |
| Anti-VCAM1 VH-CH1 | 58 | 119 |
| Anti-CD3 scFv | 5 | 66 |

TABLE 34
Components and sequences of anti-
CD3 Fab/anti-VCAM1 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL-Cκ | 6 | 67 |
| IRES | 3 | — |
| Anti-CD3 VH-CH1 | 7 | 68 |
| Anti-VCAM1 scFv | 59 | 120 |

TABLE 35
Components and sequences of anti-
VCAM1 scFv/anti-CD3 scFv BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-VCAM1 scFv | 60 | 121 |
| Anti-CD3 scFv | 18 | 79 |

TABLE 36
Components and sequences of anti-
CD3 knob/anti-VCAM1 hole BsAb

| | Nucleic Acid | Amino Acid |
|---|---|---|
| | Sequence | Sequence |
| Name | (SEQ ID NO) | (SEQ ID NO) |
| Signal Peptide | 1 | 63 |
| Anti-CD3 VL knob Ck | 10 | 71 |
| IRES | 3 | — |
| Anti-CD3 VH knob CH1-Fc | 11 | 72 |
| Anti-VCAM1 hole-Ck | 61 | 122 |
| Anti-VCAM1 VH hole CH1-Fc | 62 | 123 |

1.2 Characterization of Anti-PSMA Fab/Anti-CD3 scFv, Anti-CD3 Fab/Anti-PSMA scFv, Anti-PSMA scFv/Anti-CD3 scFv and Anti-CD3 Knob/Anti-PSMA Hole BsAbs

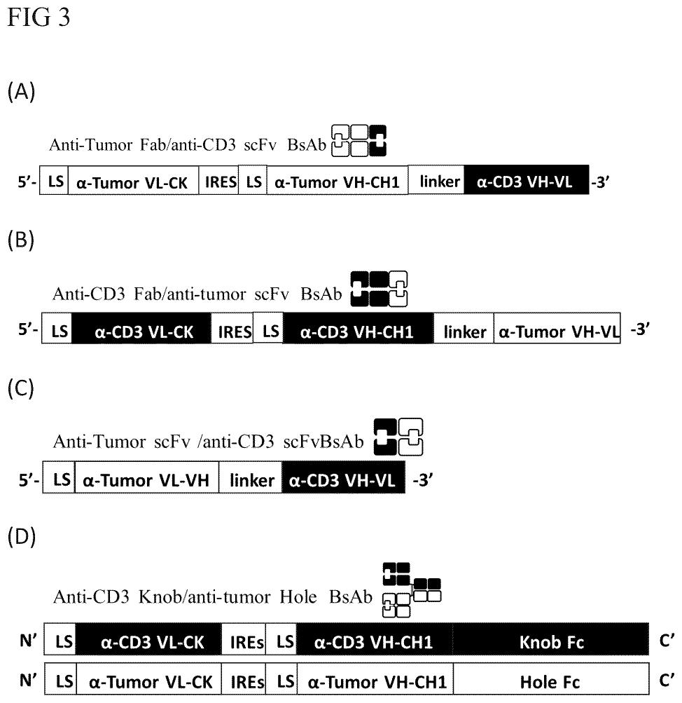

FIG. 4 depicts the SDS-PAGE analysis on anti-PSMA Fab/anti-CD3 scFv, anti-CD3 Fab/anti-PSMA scFv, anti-EGFR Fab/anti-CD3 scFv, and anti-CD3Fab/anti-PD-L1scFv BsAbs, which respectively composed of an Fab fragment about 95 kDa under reducing condition; on the other hand, a 170 kDa ant-CD3 knob/anti-PSMA hole BsAbs and a 50 kDa anti-PSMAscFv/antiCD3 scFv were respectively observed under non-reducing condition. Anti-EGFR antibody, Erbitux®, was included in the SDS PAGE as a positive control.

Bi-functional activities of the BsAbs were examined in this example by flow cytometry. Results are depicted in FIG. 3.

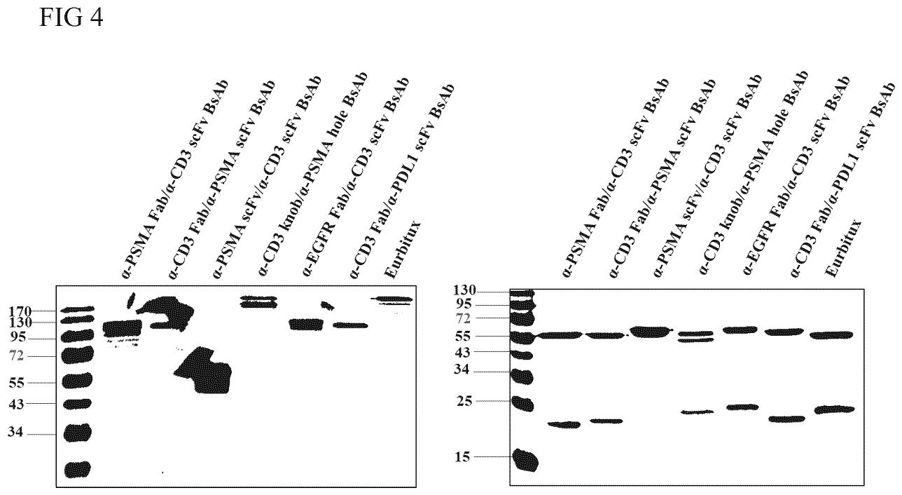

Each of the anti-PSMA Fab/anti-CD3 scFv, anti-CD3 Fab/anti-PSMA scFv, anti-PSMA scFv/anti-CD3 scFv and anti-CD3 knob/anti-PSMA hole BsAbs was capable of binding to CD3-positive T cells (i.e., Jurkat T cells) and to PSMA-positive prostate cancerous cells (i.e., LNCaP cells), while the anti-EGFR Fab/anti-CD3 scFv specifically recognized EGFR-positive cancerous cells (i.e., HT29 cells); and the anti-CD3 Fab/anti-PD-L1 scFv specifically recognized CD3-positive T cells (i.e., Jurkat T cells) and PD-L1-positive prostate cancerous cells (i.e., LNCaP cells). The data in FIG. 5 confirmed that the antibodies of Example 1 indeed possessed specificities to two different antigens.

1.3 Production of Humanized OKT3 Anti-Tumor/Anti-CD3 BsAbs

In this example, recombinant humanized OKT3 VH and VL sequences were produced in accordance with procedures described in the “Material and Methods” section. The humanized OKT3 VH and VL amino acid sequence, and respective CDRs are provided in Tables 37 and 38.

TABLE 37
Amino acid sequences of humanized OKT3 VH and its CDRs

| | Amino Acid |
|---|---|
| | Sequence |
| Name | (SEQ ID NO) |
| Anti-CD3 VH | 124 |
| HCDR1 | 125 |
| HCDR2 | 126 |
| HCDR3 | 127 |

TABLE 38
Amino acid sequences of humanized OKT3 VL and its CDRs

| | Amino Acid |
|---|---|
| | Sequence |
| Name | (SEQ ID NO) |
| Anti-CD3 VL | 128 |
| LCDR1 | 129 |
| LCDR2 | 130 |
| LCDR3 | 131 |

The humanized OKT3 VH and VL sequences could then fused with anti-tumor sequences described above in Tables 1 to 36 and thereby constructing desired humanized OKT3 anti-tumor/anti-CD3 BsAbs. Sequences of humanized OKT3 anti-CD3 scFv for constructing anti-tumor Fab/anti-CD3 scFv are provided in Table 39; sequences of humanized OKT3 anti-CD3 VL-Ck and VH-CH1 for constructing anti-CD3 Fab/anti-tumor scFv are provided in Table 40; sequences of humanized OKT3 anti-CD3 scFv for constructing anti-tumor scFv/anti-CD3 scFv are provided in Table 41; and sequences of humanized OKT3 anti-CD3 VL knob and VH knob for constructing anti-CD3 knob/anti-tumor hole are provided in Table 42.

TABLE 39
Sequences of humanized OKT3 anti-CD3 scFv for
constructing anti-tumor Fab/anti-CD3 scFv

| | Nucleic Acid | Amino Acid |
|---|---|---|
| | Sequence | Sequence |
| Name | (SEQ ID NO) | (SEQ ID NO) |
| Anti-CD3 scFv | 132 | 133 |

TABLE 40
Sequences of humanized OKT3 anti-CD3 VL-Ck and VH-
CH1 for constructing anti-CD3 Fab/anti-tumor scFv

| | Nucleic Acid | Amino Acid |
|---|---|---|
| | Sequence | Sequence |
| Name | (SEQ ID NO) | (SEQ ID NO) |
| Anti-CD3 VL-Ck | 134 | 135 |
| Anti-CD3 VH-CH1 | 136 | 137 |

TABLE 41
Sequences of humanized OKT3 anti-CD3 scFv for
constructing anti-tumor scFv/anti-CD3 scFv

| | Nucleic Acid | Amino Acid |
|---|---|---|
| | Sequence | Sequence |
| Name | (SEQ ID NO) | (SEQ ID NO) |
| Anti-CD3 scFv | 138 | 139 |

TABLE 42
Sequences of humanized OKT3 anti-CD3 VL knob and VH
knob for constructing anti-CD3 knob/anti-tumor hole

| | Nucleic Acid | Amino Acid |
|---|---|---|
| | Sequence | Sequence |
| Name | (SEQ ID NO) | (SEQ ID NO) |
| Anti-CD3 VL knob | 140 | 141 |
| Anti-CD3 VH knob CH1-Fc knob | 142 | 143 |

### Example 2 Proliferation and Characterization of CD3⁺/CD8⁺ Cells Differentiated by BsAbs of Example 1

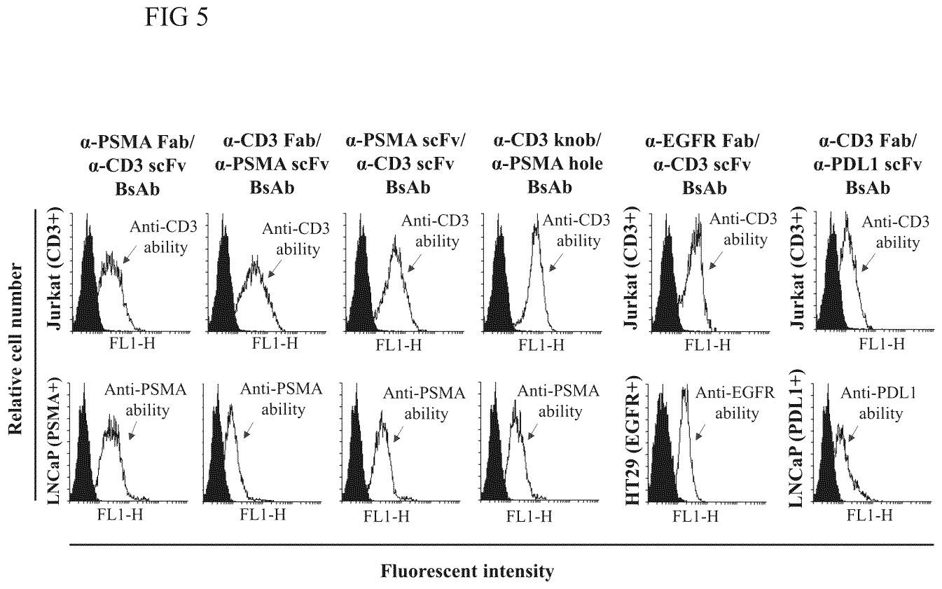

To produce T cells armed with BsAbs of Example 1, peripheral blood mononuclear cells (PBMCs) were isolated from the blood of healthy subjects, and differentiated by culturing in a media containing cytokine IL-2 plus murine monoclonal antibody OKT3 or cytokine IL-2 plus the BsAbs of Example 1.2 (i.e., anti-PSMA Fab/anti-CD3 scFv, anti-CD3 Fab/anti-PSMA scFv, anti-PSMA scFv/anti-CD3 scFv, and anti-CD3 knob/anti-PSMA hole BsAbs). The cultured cells were respectively harvested on days 7 and 14, and analyzed by flow cytometer with the aid of FITC-conjugated anti-CD3, anti-CD4, and anti-CD8 antibodies. Quantified results are summarized in Table 43 and FIG. 6.

The data in Table 43 indicates that the human BsAbs of the Example 1.2 were as effective as murine OKT3 in inducing differentiation of CD3⁺/CD8⁺ cells from PBMCs. Further, except those induced by anti-PSMA scFv/anti-CD3 scFv BsAb, the populations of CD3⁺ cells induced by the BsAbs of Example 1.2 were over 80% after 7 days in culture, and over 90% after 14 days in culture, and the total numbers of the CD3⁺/CD⁸⁺ cells were about 4-5 folds after 18 days in culture (FIG. 6).

TABLE 43
Induced differentiation of PBMCs into CD3⁺/
CD⁸⁺ cells by OKT3 or BsAbs of Example 1.2

| Day 7 | | α-PSMA Fab/ | α-CD3 Fab/ | α-PSMA scFv/ | α-CD3 knob/ |
|---|---|---|---|---|---|
| T cell | OKT3 | α-CD3 scFv | α-PSMA scFv | α-CD3 scFv | α-PSMA hole |
| population | T cell | BsAb-T cell | BsAb-T cell | BsAb-T cell | BsAb-T cell |
| CD3 (%) | 91.77 | 96.42 | 80.61 | 65.7 | 89.8 |
| CD4 (%) | 7.96 | 13.66 | 9.5 | 9.69 | 12.31 |
| CD8 (%) | 80.21 | 80.6 | 66.81 | 59.85 | 75.12 |
| Day 14 | | α-PSMA Fab/ | α-CD3 Fab/ | α-PSMA scFv/ | α-CD3 knob/ |
| T cell | OKT3 | α-CD3 scFv | α-PSMA scFv | α-CD3 scFv | α-PSMA hole |
| population | T cell | BsAb | BsAb | BsAb | BsAb |
| CD3 (%) | 92.71 | 97.8 | 92.14 | 64.75 | 95.04 |
| CD4 (%) | 2.27 | 9.19 | 2.32 | 4.29 | 14.4 |
| CD8 (%) | 73.02 | 86.4 | 72.51 | 51.95 | 85.06 |

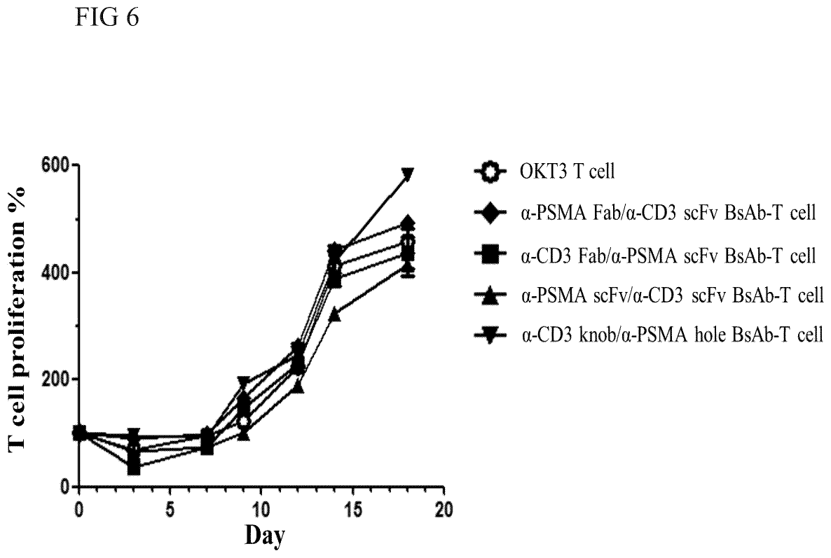

To confirm whether the differentiated T cells indeed were armed with the BsAbs of Example 1.2, the anti-antigen fragments (e.g., anti-PSMA and anti-CD3) on the surfaces of the proliferated T cells were analyzed by flow cytometer with the aid of FITC-conjugated goat anti-human Fab antibody. Results are illustrated in FIG. 7.

The data in FIG. 7 confirmed that PBMCs after cultured with the BsAbs of Example 1.2 for 14 days successfully produced PSMA-specific T cells, by arming on their surfaces the anti-PSMA fragment of BsAbs of Example 1.2.

### Example 3 Effect of T Cells of Example 2 on Cancerous Cells

3.1 Cytotoxicity Effect of T Cells of Example 2

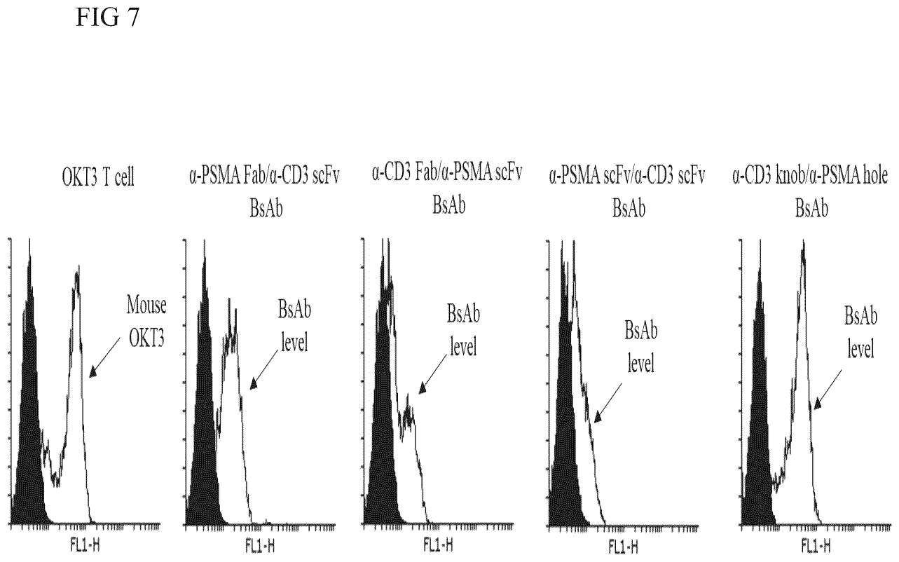

The cancer cell killing effect of CD3⁺/CD8⁺ T cells respectively differentiated by the murine OKT3 and BsAbs of Example 1.2 were evaluated in LNCaP (PSMA+) cells by CytoTox 96® Non-Radioactive Cytotoxicity Assay (Promega, G1780) in accordance with the manufacture's instruction. Results are summarized in Table 44 and FIG. 8.

It was found that about 8.4%, 11.6% and 19.3% of LNCaP cells were killed by CD3⁺/CD8⁺ T cells induced by murine OKT3 at the effect cells to target cells ratio (E/T ratio) of 3:1, 5:1 and 10:1, respectively; whereas about 65%, 87% and 97% of LNCaP cells were killed by T cells armed with anti-PSMAFab/anti-CD3scFv BsAb at the same E/T ratio. Similar improved cytotoxicity effects were also found with T cells armed with anti-CD3 Fab/anti-PSMA scFv, anti-PSMA scFv/anti-CD3 scFv, and anti-CD3 knob/anti-PSMA hole BsAbs (FIG. 8).

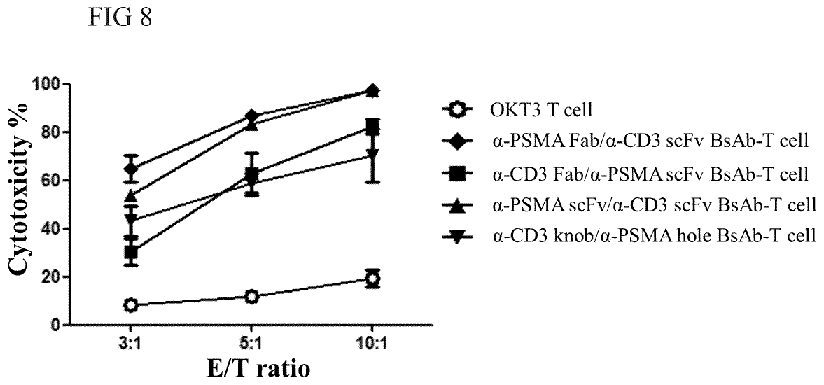

Imagine analysis further revealed that upon contacting with the tumor cells, significant portion of the T cells of Example 2 (or T cells armed with anti-CD3 Fab/anti-PSMA scFv, anti-PSMA scFv/anti-CD3 scFv, or anti-CD3 knob/anti-PSMA hole BsAbs of Example 1) bound specifically onto the surface of LNCaP cells, whereas T cells differentiated by murine OKT3 remained mostly un-bound even after incubation for 8 hrs (FIG. 9).

TABLE 44
Cytotoxic effects of T cells respectively differentiated
by murine OKT3 and BsAbs of Example 1.2

| | | α-PSMA Fab/ | α-CD3 Fab/ | α-PSMA scFv/ | α-CD3 knob/ |
|---|---|---|---|---|---|
| Effect cell/ | OKT3 | α-CD3 scFv | α-PSMA scFv | α-CD3 scFv | α-PSMA hole |
| Target cell | T cell | BsAb T cell | BsAb T cell | BsAb T cell | BsAb T cell |
| 3:1 | 8.4 ± 1.6 | 64.8 ± 4.4 | 30.3 ± 4.5 | 53.8 ± 0.5 | 43.1 ± 5.3 |
| 5:1 | 11.7 ± 1.0 | 87.0 ± 0.8 | 62.6 ± 7.0 | 83.4 ± 1.0 | 58.6 ± 3.3 |
| 10:1 | 19.4 ± 2.7 | 97.3 ± 1.3 | 82.2 ± 2.3 | 97.6 ± 1.4 | 70.4 ± 8.9 |

3.2 Characterization of the Cancer Cell Killing Efficacy of T Cells of Example 2

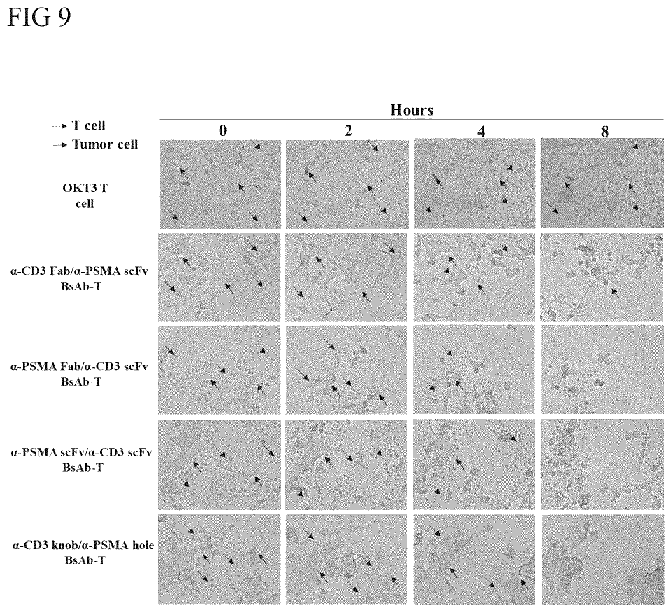

To further analyze the cancer cell killing efficacy exhibited by T cells of Example 2 (or CD3⁺/CD8⁺ T cells armed with BsAbs of Example 1), the cytokines secreted therefrom were collected and analyzed by ELISA, and the result indicated that the levels of IL-2, INF-γ, TNF-α, Granzyme B, and Perforin were all significantly higher than that secreted from T cells differentiated by murine OKT3 (FIG. 10).

3.3 Enhanced Cancer Cell-Killing Effect by the T Cells of Example 2 Further Modified with BsAbs of Example 1.2

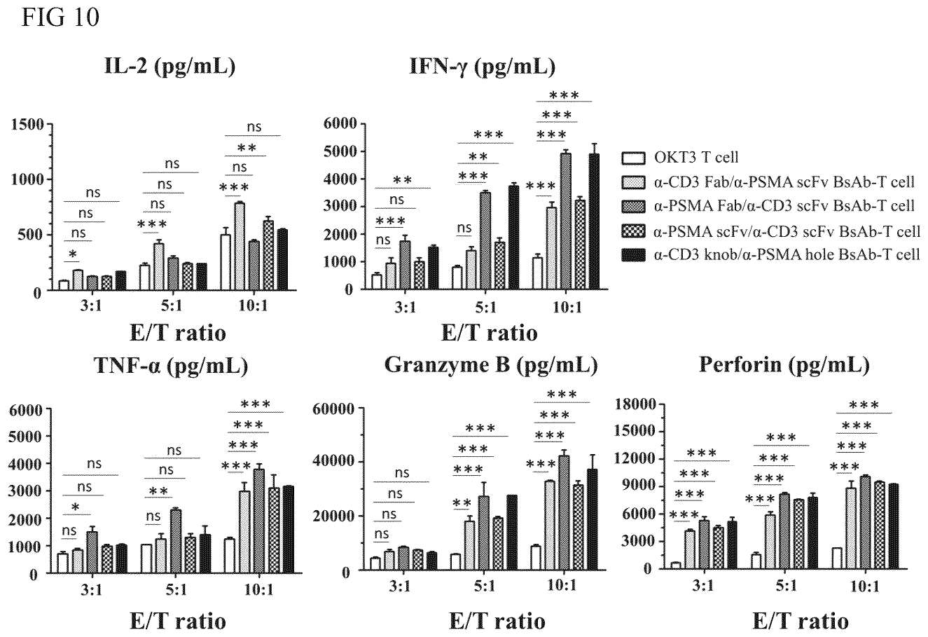

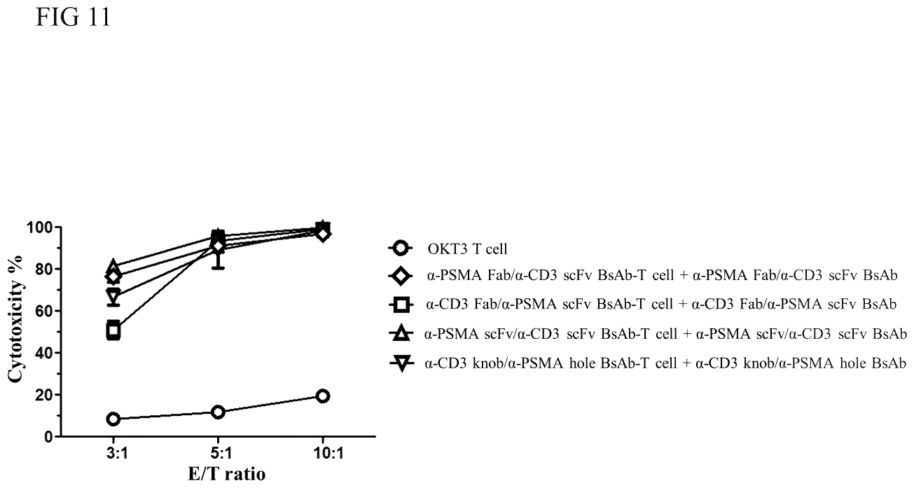

In this example, BsAbs of Example 1.2 were further added to the culture media of the T cells of Example 2 at the E/TE/T ration of 3:1, 5:1, and 10:1, and cultivated for 18 hrs, then the mixture was added to PSMA⁺ cancer cells (i.e., LNCaP cells). The cancer cell killing effect was evaluated by cytotoxicity assay as that in Example 3.1, and the levels of cytokines in the supernatant collected from the LNCaP cells were analyzed by ELISA. Results are depicted in FIGS. 11 and 12.

As depicted in FIG. 11, T cells of Example 2 further modified with BsAbs of Example 1.2 exhibited much higher cell killing efficacy, with over 90% killing effect at E/TE/T ratio of 10:1, while T cells differentiated by murine OKT3 had a meager level of about 20% at the same E/TE/T ratio. As expected, the respective cytokine levels including IL-2, INF-γ, TNF-α, Granzyme B, and Perforin were also much higher than that of the control (i.e., T cells differentiated by murine OKT3) (FIG. 12).

3.4 Effect of T Cells Armed with Anti-CD3/Anti-PD-L1 BsAbs of Example 1 on Malignant Pancreatic Cancer or Triple-Negative Breast Cancer (TNBC)

Malignant pancreatic cancer and triple-negative breast cancer (TNBC) are both infamous for their high mortalities and extremely limited chance of being cured. In this example, effects of T cells armed with anti-CD3/anti-PD-L1 BsAbs of Example 1.2 on these two infamous cancers were tested using a malignant pancreatic cell line MIA PaCa-2 and a TNBC cell line MDA-MB-231.

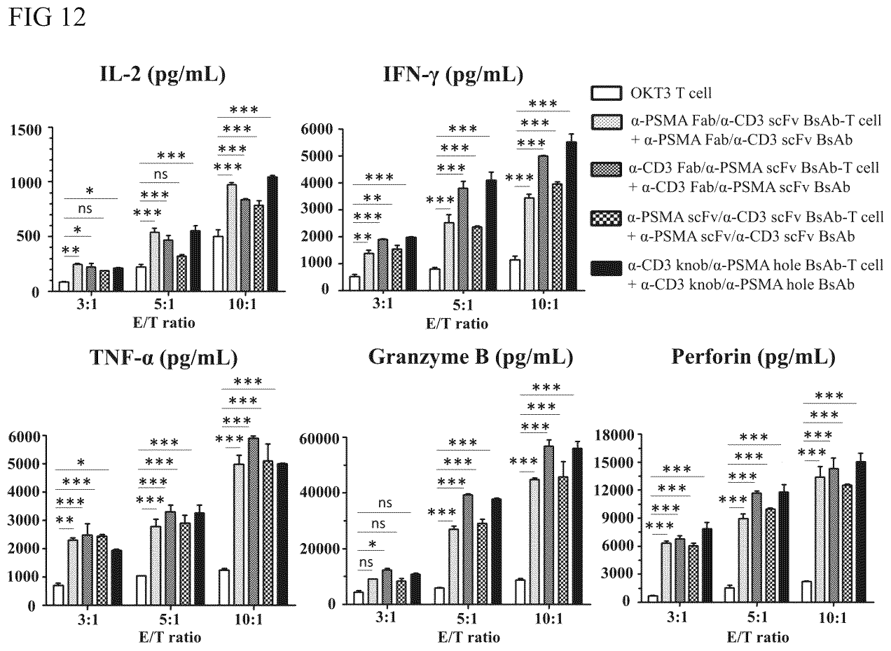

In malignant pancreatic cancer cells, T cells armed with anti-CD3/anti-PD-L1 BsAbs of Example 1.2 exhibited much higher cytotoxic effects, as compared to that of the control T cells (i.e., differentiated T cells induced by murine OKT3), and the killing efficacy was enhanced even more if the T cells were further modified with anti-CD3/anti-PD-L1 BsAbs of Example 1.2. The cancer killing effect of the T cells armed with anti-CD3/anti-PD-L1 BsAbs of Example 1.2 was found to be even more significant in TNBC cells, with nearly 100% killing effect even at a low E/T ratio of 3:1; while the control T cells exhibited merely 10% killing efficacy at the same ratio (FIG. 13).

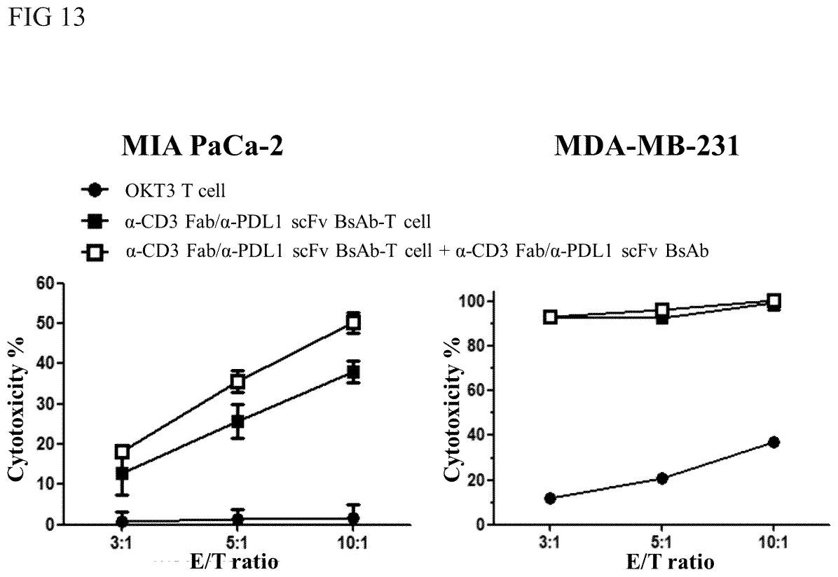

Imagine analysis revealed that T cells differentiated by murine OKT3 remained mostly un-bond to both MIA PaCa-2 and MDA-MB-231 cells, while significant portion of MIA PaCa-2 and MDA-MB-231 cells became apoptotic after incubating with T cells that were further modified with anti-CD3/anti-PD-L1 BsAbs of Example 1.2 (FIGS. 14A and 14B).

3.5 Time Effects on the Amounts of BsAbs Remained on the Surfaces of T Cells

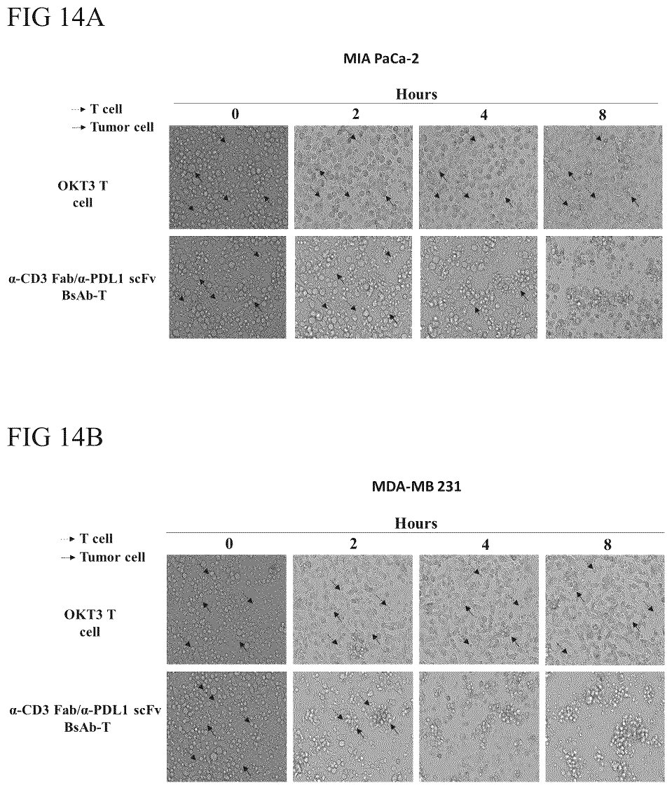

The anti-PSMA/anti-CD3 BsAbs of Example 1.2 were incubated with serum for various periods of time (i.e., 0, 24, 48 and 72 hrs), and analyzed by flow cytometry to evaluate the residual amounts of the BsAbs remain on the surfaces of T cells. Results are illustrated in FIG. 15.

It appeared that the amounts of BsAbs on the surface of the T cells declined with the time, among the four types or structures of BsAbs, anti-CD3 knob/anti-PSMA hole BsAb was least affected by degradation, about 47% of BsAb still remained on the T cell surface after 72 hrs, while the level of the anti-PSMA scFv/anti-CD3 scFv remained on the T cell surface dropped to a low level of 15%.

### Example 4 Effects of Murine OKT3 T Cells Modified with BsAbs of Example 1.2

As the finding provided in Examples 1 to 4, murine OKT3 differentiated T cells were not as effective as the T cells of Example 2 (i.e., T cells armed with BsAbs of Example 1) in terms of the cancer killing effect, due to their low binding affinity to cancer cells and low levels of cytokines secreted therefrom. Accordingly, in this example, murine OKT3 T cells were modified by co-incubating with BsAbs of Example 1.2, then their cytotoxic effects in cancer cells and were evaluated.

4.1 Murine OKT3 T Cells Modified with BsAbs of Example 1.2

To modify the murine OKT3 T cells, about 3×10⁵ murine OKT3 T cells were mixed with various amounts (8, 24, 120, 600 and 3,000 ng) of anti-PSMA/anti-CD3 BsAbs of example 1.2 and analyzed by flow cytometry. Results indicated that about 600 ng BsAbs of example 1.2 was sufficient to load the surfaces of murine OKT3 T cells with BsAbs of example 1.2 (data not shown).

4.2 Effects of Murine OKT3 T Cells Modified with BsAbs of Example 1.2 on PSMA⁺ Cells

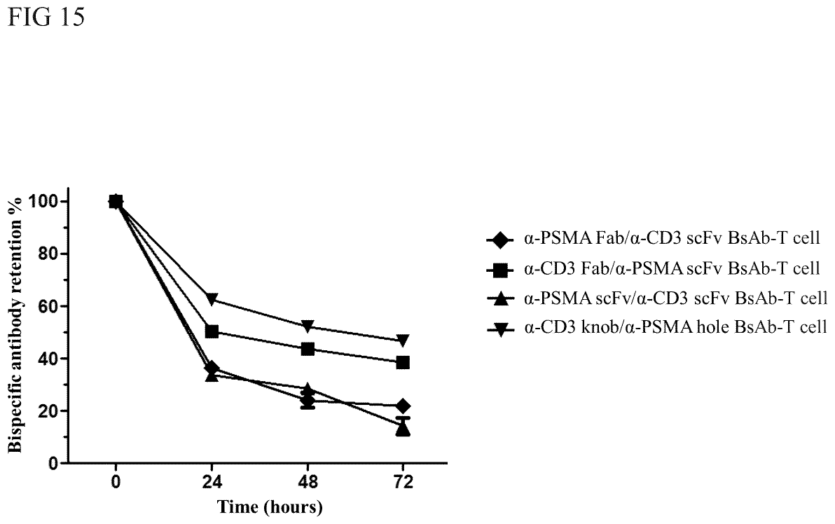

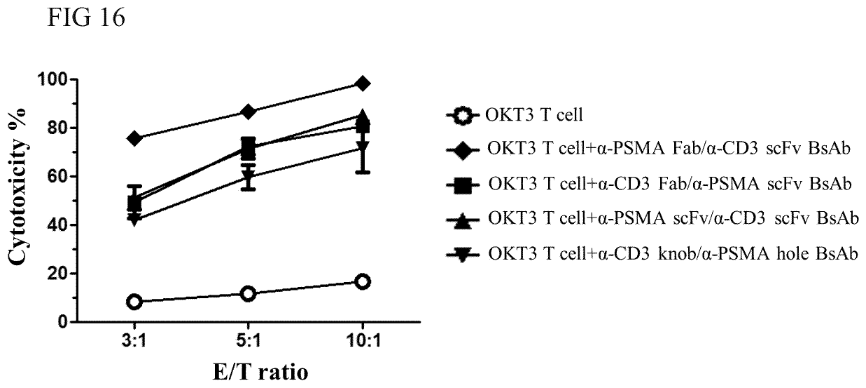

As expected, the cancer cell killing ability of murine OKT3 differentiated T cells toward LNCaP cells (PSMA⁺) increased significantly after being modified with the BsAbs of Example 1, including anti-PSMA Fab/anti-CD3 scFv, anti-CD3 Fab/anti-PSMA scFv, anti-PSMA scFv/anti-CD3 scFv, and anti-CD3 knob/anti-PSMA hole, as compared to that before modification. Among which, murine OKT3 T cells modified with anti-PSMA Fab/anti-CD3 scFv exhibited near 100% cell killing effect at E/T ratio of 10:1 (FIG. 16). The respective cytokine levels including IL-2, INF-γ, TNF-α, Granzyme B, and Perforin secreted from murine OKT3 T cells modified with the BsAbs of Example 1.2 also increased significantly, compared with those of the control unmodified T cells (FIG. 17).

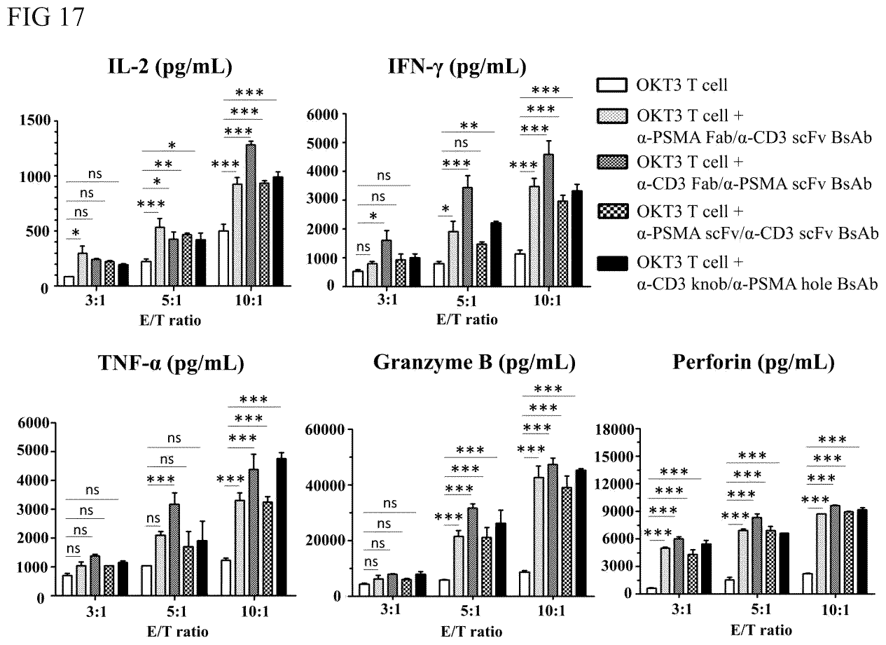

Imagine analysis revealed that murine OKT3 T cells modified with the anti-PSMA Fab/anti-CD3 scFv, anti-CD3 Fab/anti-PSMA scFv, anti-PSMA scFv/anti-CD3 scFv, or anti-CD3 knob/anti-PSMA hole BsAbs were more specifically bound o the surfaces of LNCaP cells, as compared with that of the unmodified OKT3 T cells (FIG. 18).

4.3 Effects of Murine OKT3 T Cells Modified with BsAbs of Example 1.2 on EGFR⁺ Cells

In this example, murine OKT3 T cells were modified with anti-EGFR Fab/anti-CD3 scFv BsAb of Example 1.2, and subjected to analysis to evaluate their capability in killing EGFR⁺ cancer cells (i.e., HT29 cells).

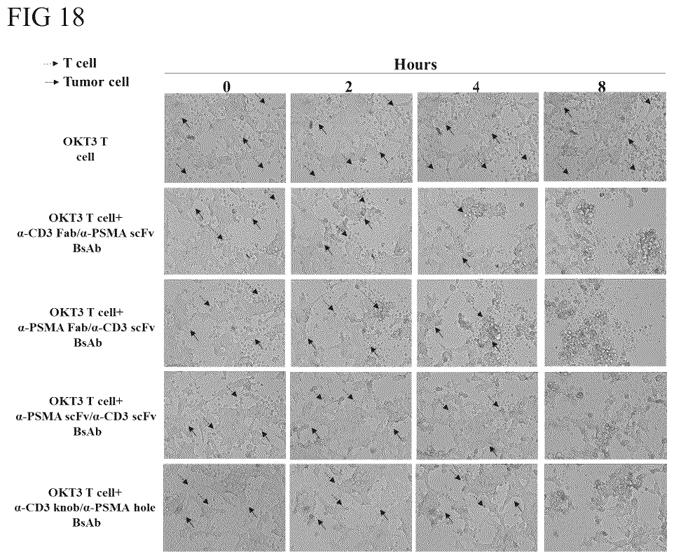

Similar to finding in Example 5.1, after being modified with anti-EGFR Fab/anti-CD3 scFv BsAb of Example 1.2, murine OKT3 T cells exhibited an enhanced cytotoxic effect, with about 50% cell killing effect at the E/T ratio of 10:1 (FIG. 19A), and enhanced cytokine levels in INF-γ, TNF-α, Granzyme B, and Perforin (FIG. 19B).

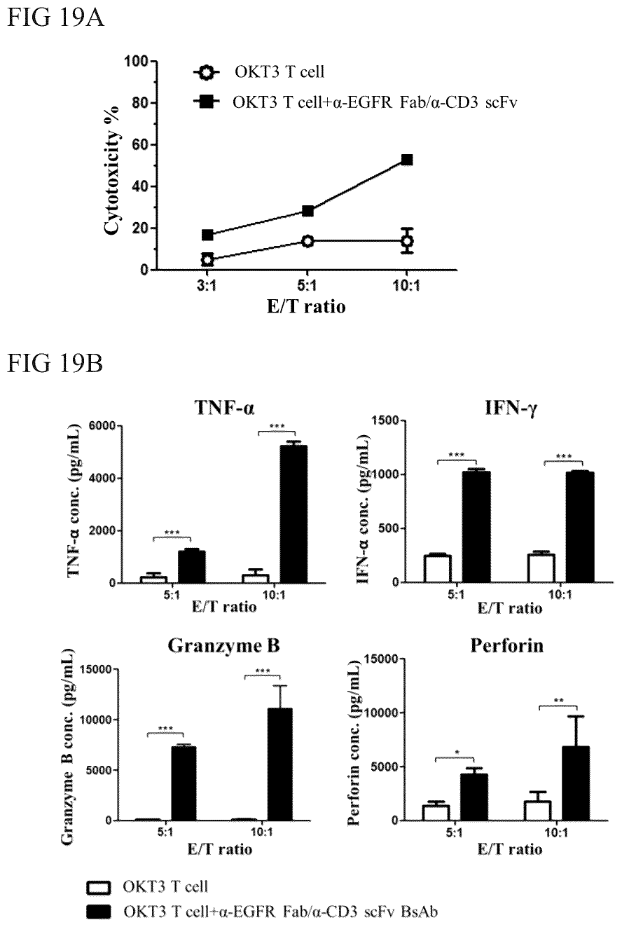

Imagine analysis revealed that murine OKT3 T cells modified with the anti-EGFR Fab/anti-CD3 scFv BsAbs were more specifically bound o the surfaces of HT29 cells, as compared with that of the unmodified OKT3 T cells (FIG. 20).

4.4 Effects of Murine OKT3 T Cells Modified with BsAbs of Example 1.2 on PD-L1⁺ Cells

In this example, murine OKT3 T cells were modified with anti-CD3 Fab/anti-PD-L1 scFv BsAb of Example 1.2, and subjected to analysis to evaluate their capability in killing PD-L1⁺ cancer cells (i.e., LNCaP cells).

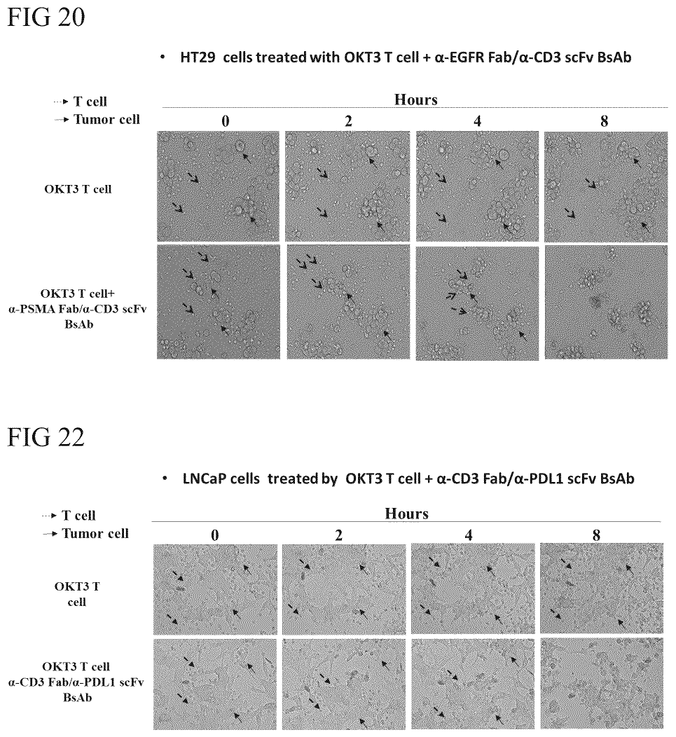

Similar to findings in Example 5.1 and 5.2, after being modified with anti-CD3 Fab/anti-PD-L1 scFv BsAb of Example 1.2, murine OKT3 T cells exhibited an enhanced cytotoxic effect, with about 92% cell killing effect at the E/T ratio of 10:1 (FIG. 21A), and enhanced cytokine levels in INF-γ, TNF-α, Granzyme B, and Perforin (FIG. 21B).

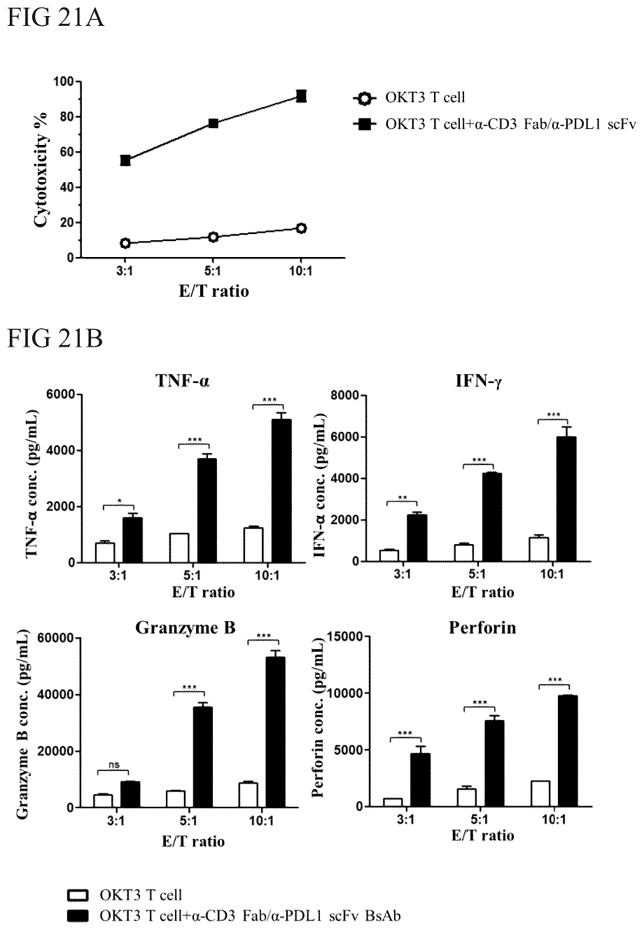

Imagine analysis revealed that murine OKT3 T cells modified with the anti-CD3 Fab/anti-PD-L1 scFv BsAb were more specifically bound o the surfaces of LNCaP cells, as compared with that of the unmodified OKT3 T cells (FIG. 22).

4.5 Effects of Murine OKT3 T Cells Modified with Anti-CD3 Fab/Anti-PD-L1 scFv of Example 1.2 on Malignant Pancreatic Cancer Cells and TNBC Cells

In this example, the cancer killing effect of murine OKT3 T cells modified with anti-CD3 Fab/anti-PD-L1 scFv BsAbs of Example 1.2 were tested on malignant pancreatic cancer cell line MIA PaCa-2 (PD-L1⁺ cells) and TNBC cells (MDA-MB-231 cells) in accordance with similar procedures described in Example 3.4.

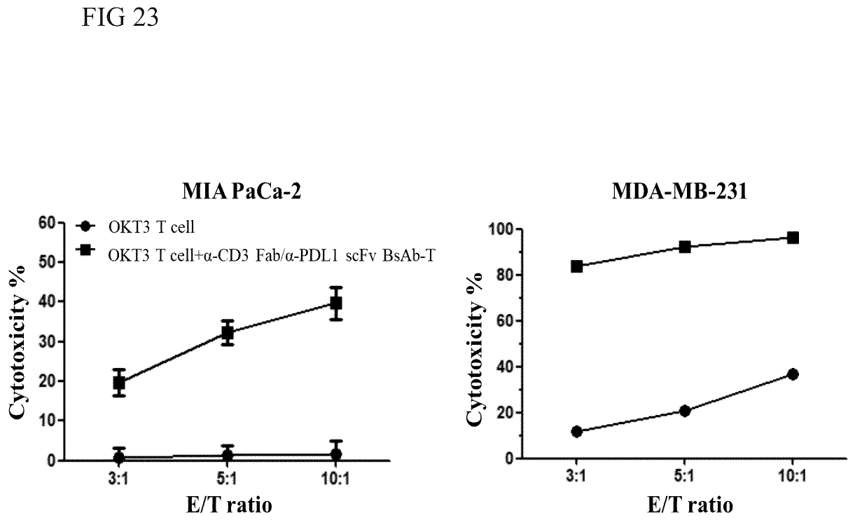

In malignant pancreatic cancer cells, murine OKT3 T cells modified with anti-CD3Fab/anti-PD-L1 scFv BsAbs of Example 1 exhibited much higher cytotoxic effect (about 40% cancer cells were killed), as compared to that of the control T cells (i.e., unmodified murine OKT3 T cells). The cancer killing effect was more significant in TNBC cells, with nearly 80% cancer cells were killed even at a low E/T ratio of 3:1; while the control T cells exhibited merely 20-30% killing efficacy at the same ratio (FIG. 23).

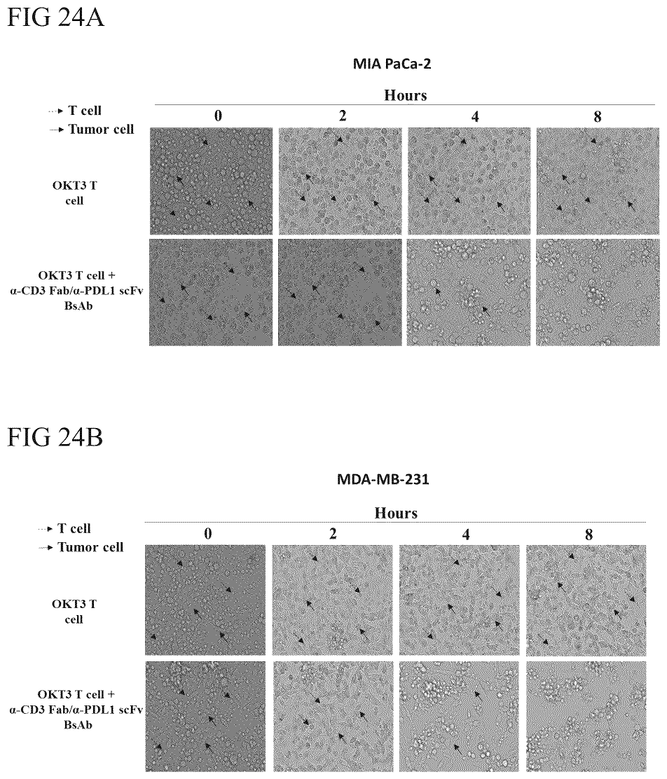

Imagine analysis revealed that murine OKT3 T cells modified with the anti-CD3 Fab/anti-PD-L1 scFv BsAb specifically bound o the surfaces of MIA PaCa-2 cells (FIG. 24A) and MDA-MB-231 cells (FIG. 24B), and resulted in enhanced cancer cell apoptosis, as compared with that of the unmodified OKT3 T cells.

### Example 5 In Vivo Effects of Murine OKT3 T Cells Modified with BsAbs of Example 1

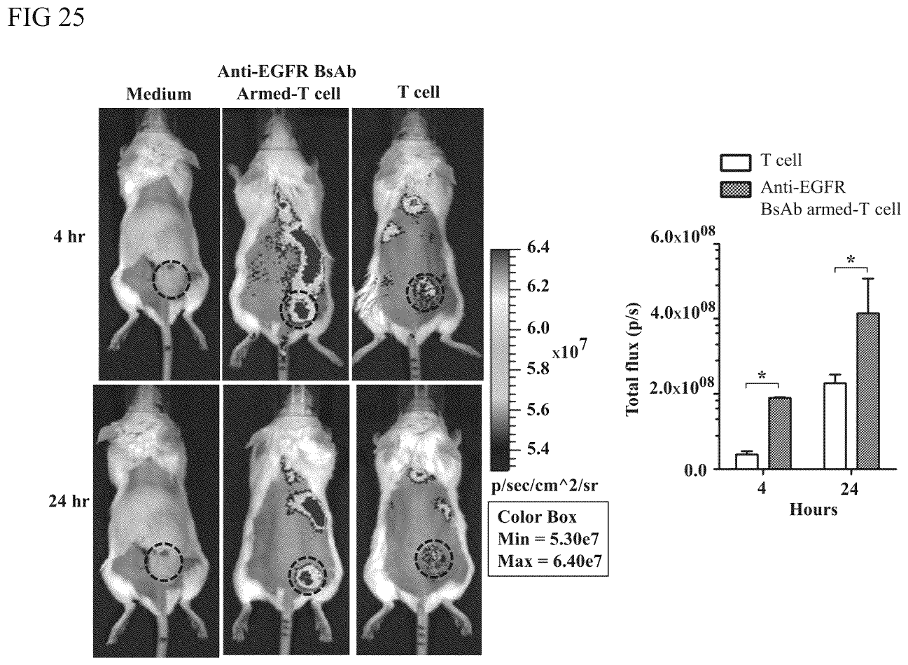

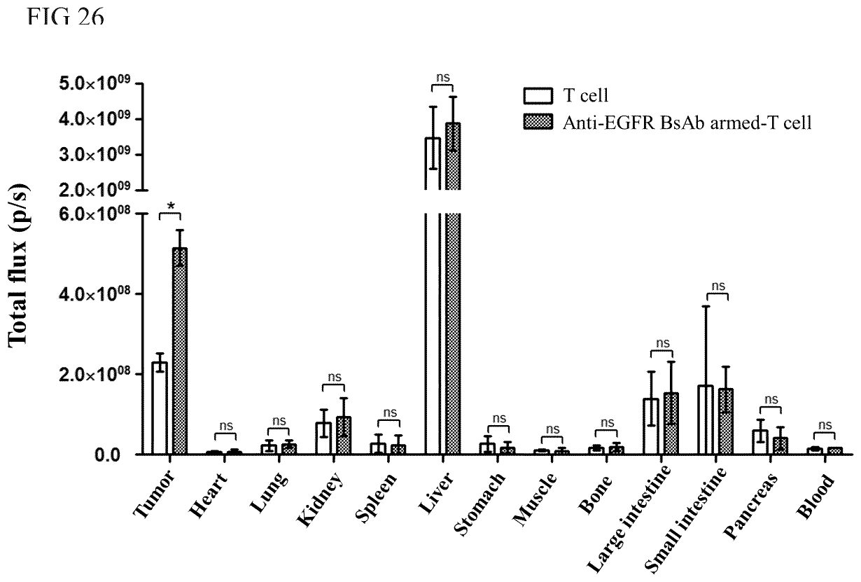

To investigate whether the BsAbs of Example 1 could improve the targeting effect of the murine OKT3 T cells, the murine OKT3 T cells were modified with anti-EGFR/anti-CD3 BsAb of Example 1.2, while at the same time labeling with a fluorescent indicator—NIR797. Then, the modified and labeled murine OKT3 T cells were injected into SCID mice bearing a heterogeneous EGFR⁺ tumor (HT29 cells) through IV injection, and live images were taken respectively at 4 and 24 hrs using IVIS imaging system. The animals were sacrificed after 24 hrs, and the tumor per se and organs including heart, lung, kidney, spleen, liver, stomach, muscle, bone, large intestine, small intestine, pancreas, and blood, were harvested and analyzed by IVIS imaging system, respectively. Results are depicted in FIGS. 25 and 26.

The photos in FIG. 25 revealed that fluorescent intensity in animals treated with the modified OKT3 T cells was found mainly concentrated on the tumor site, as compared to that treated with un-modified murine OKT3 T cells. The result confirmed that the murine OKT3 T cells modified with BsAbs of example 1 were indeed being targeted to the tumor, thereby resulted in an enhanced fluorescent intensity at the tumor site.

Further, the modified T cells were found to be concentrated in the EGFR⁺ tumor per se, while the in vivo distribution pattern of these modified T cells was similar to that of a normal healthy animal (FIG. 26).

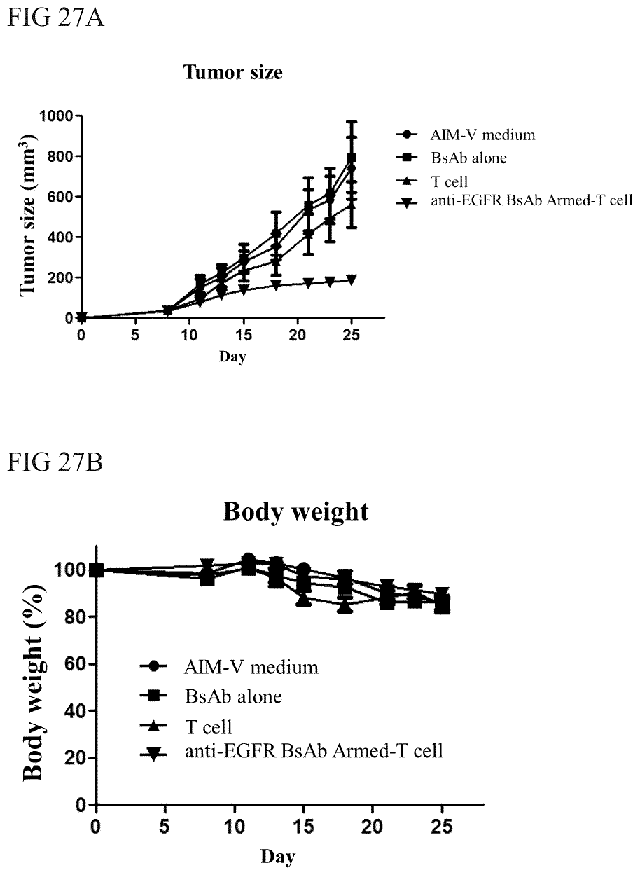

In addition, the tumor size and its weight were suppressed significantly when treated with the modified murine OKT3 T cells (FIG. 27, panels A and C), while the body weight of the animals decreased only slightly along the 25-days treatment time course (FIG. 27, panel B).

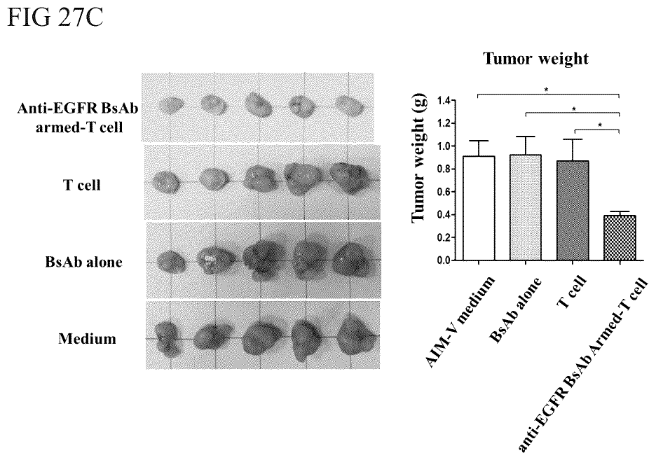

The tumors harvested from the animals were further analyzed by immunohistochemical (IHC) staining and H&E staining, and results are depicted in FIG. 28. It was found that the modified murine OKT3 T cells were mainly concentrated in the neighboring area of the tumor.

### Example 6 Comparable Studies on T Cells of Example 2 and the Modified Murine OKT3 Cells of Example 4

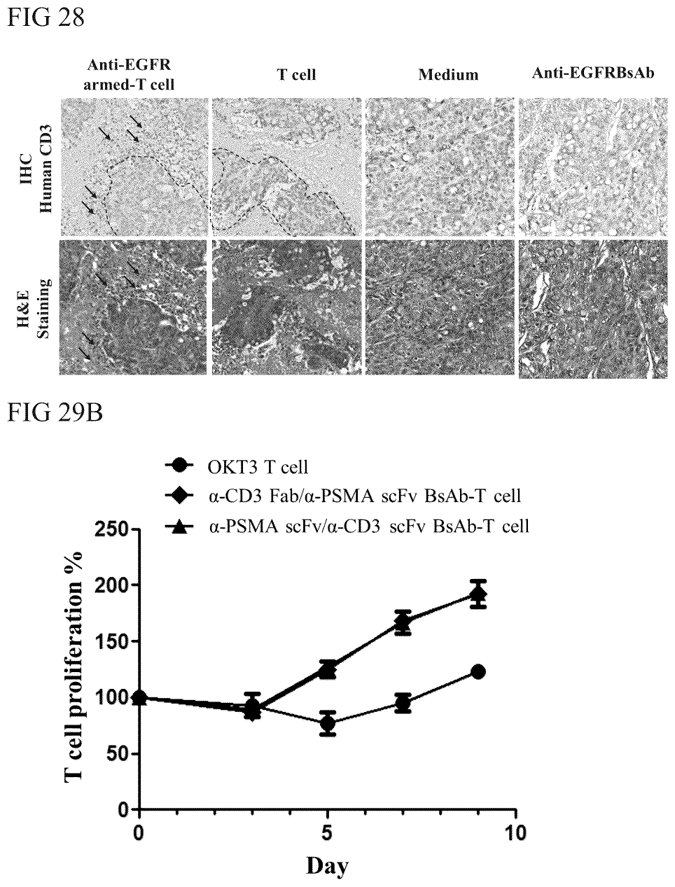

In this example, the differentiation and proliferation of T cells induced by the BsAbs of Example 1.2 (i.e., anti-PSMA Fab/anti-CD3 scFv and anti-PSMA scFv/anti-CD3 scFv) and murine OKT3 were compared. Results are depicted in FIGS. 29 to 32.

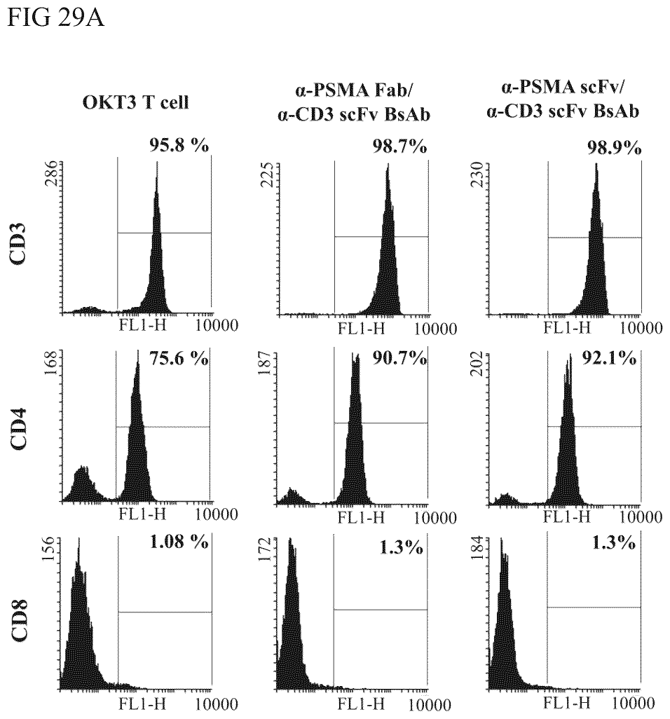

As depicted in FIG. 29A, both the BsAbs of Example 1.2 (i.e., anti-PSMA Fab/anti-CD3 scFv and anti-PSMA scFv/anti-CD3 scFv) and murine OKT3 were capable of inducing differentiation of PBMCs into CD3⁺ cells, with over 95% of cells being CD3⁺ cells. As to the CD4⁺ cells, the BsAbs of Example 1.2 (i.e., anti-PSMA Fab/anti-CD3 scFv and anti-PSMA scFv/anti-CD3 scFv) were more effective than murine OKT3 in inducing the differentiation of PBMCs into CD4⁺ T cells, in which over 90% of PBMCs were differentiated into CD4+ cells after being treated with BsAbs of Example 1.2, while only about 75% PBMCs turned into CD4⁺ cells after murine OKT3 treatment. Further, the proliferation ratio of T cells induced by BsAbs of Example 1.2 was also higher than that by murine OKT3 T cells (FIG. 29B). Flow cytometry analysis further confirmed that similar to T cells induced by murine OKT3 Abs, which appeared on the surfaces of OKT3 T cells; the two types antibody fragments carried by each BsAbs of Example 1.2 also appeared on the surfaces of the T cells induced therefrom (FIG. 30).

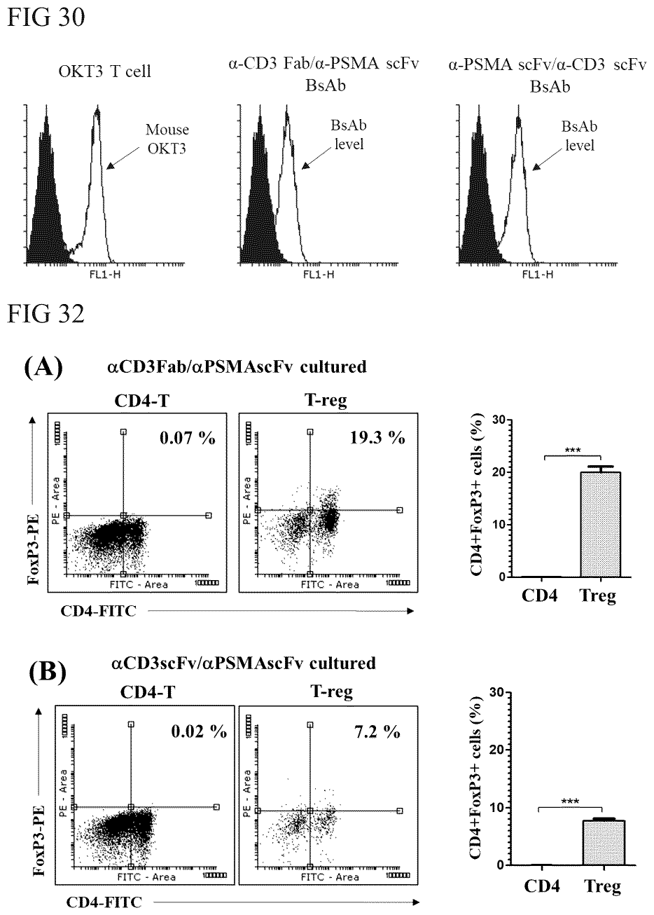

The comparative study on the cytotoxicity of murine OKT3 T cells and T cells of example 2 is provided in FIG. 31. As depicted, OKT3 T cells were incapable of killing PSMA⁺ cells (LNCaP cells), whereas the T cells of example 2 (or T cells induced by BsAbs of example 1) exhibited at least 50% cytotoxicity, and the cytotoxicity of the T cells would increase further if they were further modified with corresponding BsAbs of example 1.2. For example, the cytotoxicity of T cells differentiated by anti-CD3 Fab/anti-PSMA scFv was independently about 51.32% and 60.97% at the E/T ratio of 5:1 and 10:1; which increased to about 78.15% and 81.75% at the E/T ratio of 5:1 and 10:1, if they were further modified with anti-CD3 Fab/anti-PSMA scFv. Similar observation were found for T cells induced by anti-PSMA scFv/anti-CD3 scFv.

### Example 7 One-Step Differentiation, Proliferation of Regulatory T Cells by BsAbs of Example 1.2

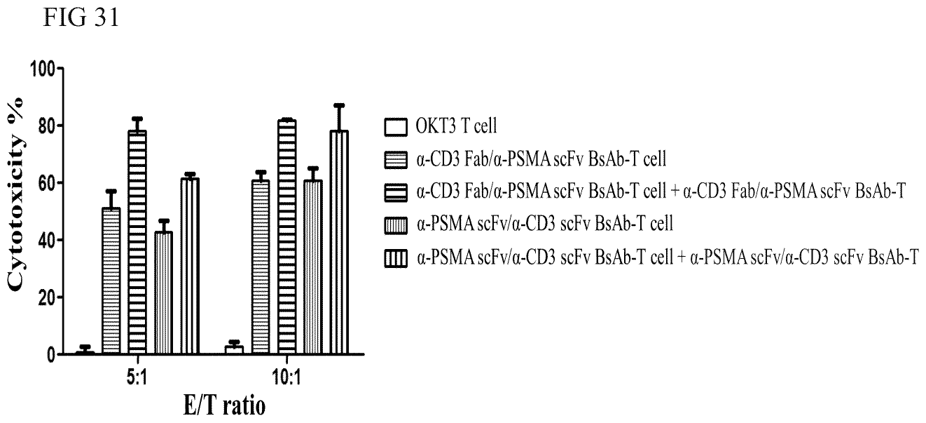

In this example, regulatory T cells were induced and formed by use of BsAbs of Example 1.2. Briefly, PBMCs from healthy human subjects were isolated and cultured with anti-CD3 Fab/anti-PSMA scFv or anti-PSMA scFv/anti-CD3 scFv of Example 1.2 for 7 days, in either case, the culture medium also contained IL-2, TGF-β, and anti-CD28 antibodies. Then, differentiated T cells were harvested and subjected to flow cytometry analysis. Results are depicted in FIG. 32.

The data indicated that anti-CD3 Fab/anti-PSMA scFv BsAb and anti-PSMA scFv/anti-CD3 scFv could respectively induce the formation of about 19.3% (FIG. 32, panel A) and 7.2% (FIG. 32, panel B) of CD4⁺FoxP3⁺ regulator T cells.

### Example 8 Characterization of Anti-EGFR/Anti-CD3 BsAb

8.1 Expression and Binding Specificity

Two anti-EGFR/anti-CD3 BsAbs, i.e., the anti-EGFR Fab/anti-CD3 scFv BsAb and anti-EGFR scFv/anti-CD3 Fab BsAb respectively described in Tables 5 and 6 of Example 1.1, were produced by Expi-293™ expression system. After confirming the expression by reducing and non-reducing SDS-PAGE analysis, bi-functional activities of the BsAbs were examined by flow cytometry. The data demonstrated that each of the anti-EGFR Fab/anti-CD3 scFv BsAb and anti-EGFR scFv/anti-CD3 Fab BsAb was capable of binding to CD3-positive T cells (i.e., Jurkat T cells) and to EGFR-positive colorectal cancerous cells (i.e., HT-29 cells) (data not shown). On the other hand, neither BsAbs recognized and bound to EGFR-negative cells (i.e., NIH/3T3 cells) (data not shown). The data demonstrated that the anti-EGFR Fab/anti-CD3 scFv BsAb and anti-EGFR scFv/anti-CD3 Fab BsAb possessed binding specificities towards two different antigens (i.e., CD3 and EGFR).

8.2 Effect of Anti-EGFR/Anti-CD3 BsAb on T Cells

To produce T cells armed with the present anti-EGFR/anti-CD3 BsAb, PBMCs were isolated from the blood of healthy subjects, and cultured in the medium supplemented with cytokine IL-2 in the presence of murine monoclonal antibody OKT3 or anti-EGFR/anti-CD3 BsAb. The PBMC thus cultured was used as a control group in this study. The cultured cells were respectively harvested on day 7 and day 14, and analyzed by flow cytometer with the aid of FITC-conjugated anti-CD3, anti-CD4, and anit-CD8 antibodies. Quantified results were summarized in Table 45. The data of Table 45 indicated that each of the anti-EGFR Fab/anti-CD3 scFv BsAb and anti-EGFR scFv/anti-CD3 Fab BsAb was effective as murine monoclonal antibody OKT3 in inducing the differentiation of CD3⁺/CD8⁺ cells from PBMC.

TABLE 45
Induced differentiation of CD3⁺/CD8⁺ cells from PBMCs
by OKT3 or anti-EGFR/anti-CD3 BsAb

| | | anti-EGFR | anti-EGFR |
|---|---|---|---|
| Day 7 | OKT3-T | Fab/anti-CD3 | scFv/anti-CD3 |
| T cell population | cells | scFv BsAb-T cells | Fab BsAb-T cell |
| CD3 (%) | 98.5 | 80.6 | 96.4 |
| CD4 (%) | 3.6 | 18.6 | 6.1 |
| CD8 (%) | 86 | 72.8 | 83.6 |
| | | anti-EGFR | anti-EGFR |
| Day 14 | OKT3-T | Fab/anti-CD3 | scFv/anti-CD3 |
| T cell population | cells | scFv BsAb-T cells | Fab BsAb-T cell |
| CD3 (%) | 98.2 | 97 | 97.2 |
| CD4 (%) | 4.8 | 4.2 | 4.1 |
| CD8 (%) | 89.2 | 86.2 | 88.8 |

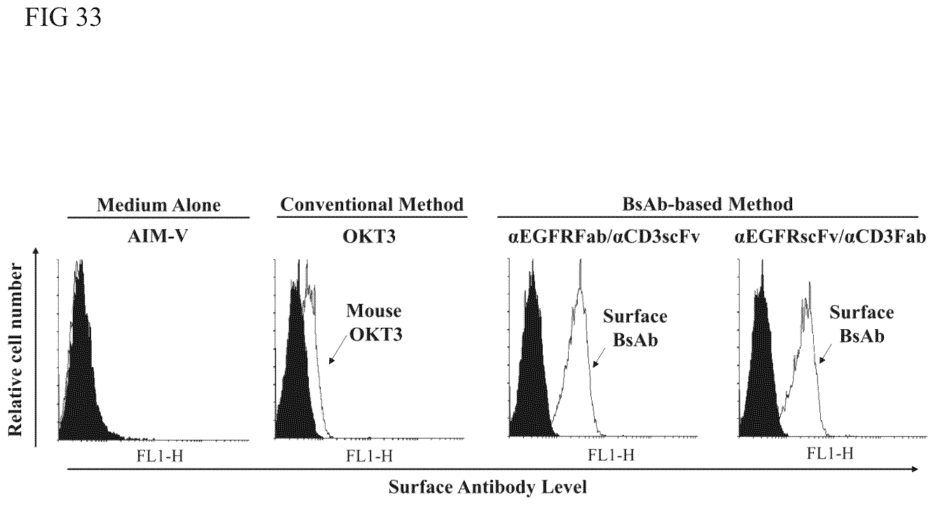

To confirm whether the differentiated T cells were armed with the present anti-EGFR/anti-CD3 BsAb, the cultured cells harvested on day 14 were analyzed by flow cytometer with FITC-conjugated goat anti-human IgG Fab antibody or FITC-conjugated goat anti-mouse IgG antibody. The data of FIG. 33 demonstrated that EGFR-specific T cells were successfully produced via cultivating the PBMCs with the present anti-EGFR/anti-CD3 BsAb (i.e., the anti-EGFR Fab/anti-CD3 scFv BsAb or anti-EGFR scFv/anti-CD3 Fab BsAb) for 14 days, in which the surfaces of T cells are armed with the anti-EGFR fragment of BsAb, i.e., in the form of anti-EGFR/anti-CD3 BsAb-armed T cells.

8.3 Cytotoxicity Effect of Anti-EGFR/Anti-CD3 BsAb-Armed T Cells

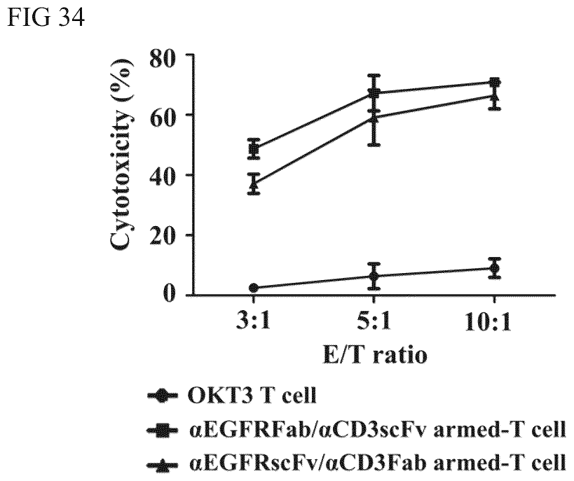

The cancer cell killing effect of the OKT3-armed CD3⁺/CD8⁺ T cells (i.e., the T cells induced by the murine OKT3 antibody) or the anti-EGFR/anti-CD3 BsAb-armed CD3⁺/CD8⁺ T cells (i.e., the T cells induced by the anti-EGFR Fab/anti-CD3 scFv BsAb or anti-EGFR scFv/anti-CD3 Fab BsAb) as described in Examples 8.2 was evaluated in HT-29 (EGFR-positive) cells. In brief, the OKT3-armed CD3⁺/CD8⁺ T cells or anti-EGFR/anti-CD3 BsAb-armed CD3⁺/CD8⁺ T cells were mixed with EGFR⁺ colon cancer cells (HT29) at different ratios (Effector cell/Target cell ratio=3:1, 5:1 or 10:1) for 18 hours; and the cancer cell viability of each groups was then determined by using non-radioactive cytotoxicity assay. The results of FIG. 34 demonstrated that the anti-EGFR/anti-CD3 BsAb-armed CD3⁺/CD8⁺ T cells (including the anti-EGFR Fab/anti-CD3 scFv BsAb-armed CD3⁺/CD8⁺ T cells, and the anti-EGFR scFv/anti-CD3 Fab BsAb-armed CD3⁺/CD8⁺ T cells) exhibited cytotoxicity effects on EGFR-positive cancer cells. Of note, the targeting and killing effects of the present anti-EGFR/anti-CD3 BsAb-armed CD3⁺/CD8⁺ T cells were superior than that of OKT3-armed CD3⁺/CD8⁺ T cells (FIG. 34).

Image analysis further confirmed that once contacting with the cancer cells, the anti-EGFR/anti-CD3 BsAb-armed T cells bound specifically onto the surface of HT-29 cells, whereas OKT3-armed T cells remained mostly un-bound even after incubation for 8 hours (data not shown).

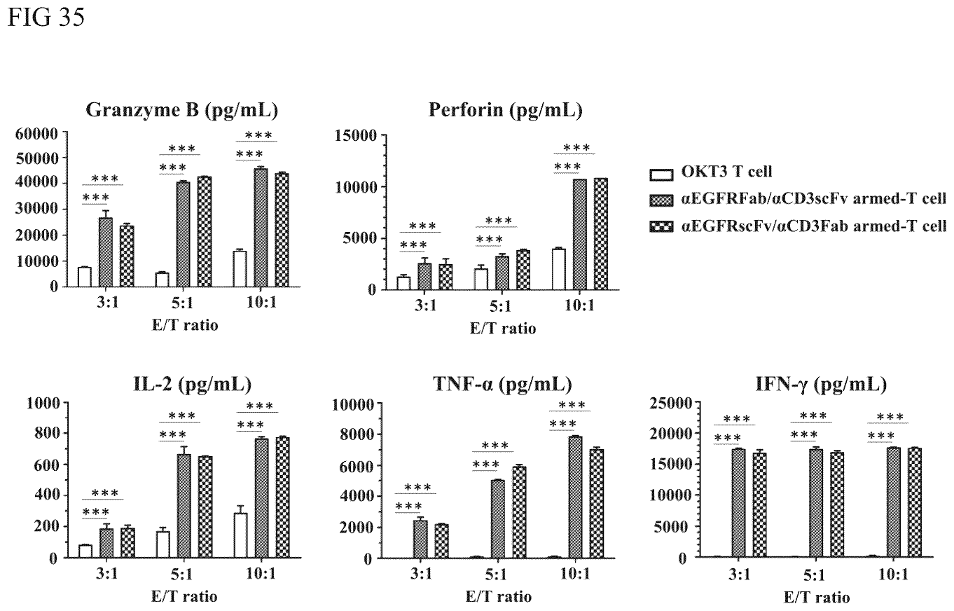

To further evaluate the cancer cell killing effect exhibited by the anti-EGFR/anti-CD3 BsAb-armed T cells, cytokines secreted therefrom were collected and analyzed by ELISA. The results indicated that the levels of Granzyme B, Perforin, IL-2, TNF-α, and INF-γ secreted from the anti-EGFR/anti-CD3 BsAb-armed T cells (including the anti-EGFR Fab/anti-CD3 scFv BsAb-armed T cells, and the anti-EGFR scFv/anti-CD3 Fab BsAb-armed T cells) were all significantly higher than those secreted from the OKT3-armed T cells (FIG. 35).

8.4 Cytotoxicity Effect of T Cells Armed with Different Levels of BsAb

To determine whether the level of BsAb armed on the surface of T cells would affect the cytotoxicity of the T cells, anti-EGFR Fab/anti-CD3 scFv BsAb or anti-EGFR scFv/anti-CD3 Fab BsAb were incubated with T cells at various doses (4.8 ng, 24 ng, 120 ng, 600 ng, or 3,000 ng) for 1 hour. The incubated cells were first detected by flow cytometry using rabbit anti-his tag antibody and FITC-conjugated goat anti-rabbit antibody to determine the levels of BsAb on the surface of T cells. It was found that the present anti-EGFR/anti-CD3 BsAbs (i.e., anti-EGFR Fab/anti-CD3 scFv BsAb or anti-EGFR scFv/anti-CD3 Fab BsAb) could be loaded on the surface of T cells in a dose-dependent manner (from about 5.6% to about 100%; data not shown).

Then, the cytotoxicity of T cells armed with different levels of anti-EGFR/anti-CD3 BsAb were mixed with EGFR⁺ colon cancer cells (HT29) at different ratios (Effector cell/Target cell ratio=3:1, 5:1 or 10:1) for 18 hours; and the cancer cell viability of each group was then determined by non-radioactive cytotoxicity assay. The results were summarized in Tables 46 and 47.

TABLE 46
Cytotoxicity of T cells armed with different levels of anti-EGFR
Fab/anti-CD3 scFv BsAb towards HT-29 cells

| BsAb modified | Anti-EGFR Fab/anti-CD3 scFv BsAb-armed T cell | | | |
|---|---|---|---|---|
| level (%) | 0% | 5.6% | 23.4% | 100% |
| E:T ratio = 3:1 | 2.51 ± 0.81 | 49.81 ± 1.77 | 48.55 ± 3.16 | 51.58 ± 6.12 |
| E:T ratio = 5:1 | 6.35 ± 4.13 | 65.80 ± 3.38 | 65.29 ± 6.39 | 70.52 ± 0.43 |
| E:T ratio = 10:1 | 9.07 ± 3.08 | 68.71 ± 3.41 | 69.37 ± 1.44 | 70.87 ± 1.06 |

TABLE 47
Cytotoxicity of T cells armed with different levels of anti-EGFR
scFv/anti-CD3 Fab BsAb towards HT-29 cells

| BsAb modified | Anti-EGFR scFv/anti-CD3 Fab BsAb-armed T cell | | | |
|---|---|---|---|---|
| level (%) | 0% | 15.3% | 47.8% | 100% |
| E:T ratio = 3:1 | 0.52 ± 2.13 | 42.03 ± 8.62 | 47.84 ± 5.39 | 47.12 ± 6.86 |
| E:T ratio = 5:1 | 2.30 ± 1.60 | 60.61 ± 3.94 | 61.96 ± 3.43 | 69.10 ± 1.64 |
| E:T ratio = 10:1 | 4.69 ± 3.32 | 67.94 ± 6.42 | 71.84 ± 1.12 | 69.73 ± 3.28 |

The data of Tables 46 and 47 demonstrated that the T cells armed with 5.6% of the present anti-EGFR/anti-CD3 BsAb on their surfaces were sufficient to target and kill the EGFR-positive cancer cells.

8.5 In Vivo Effects of Anti-EGFR/Anti-CD3 BsAb-Armed T Cells

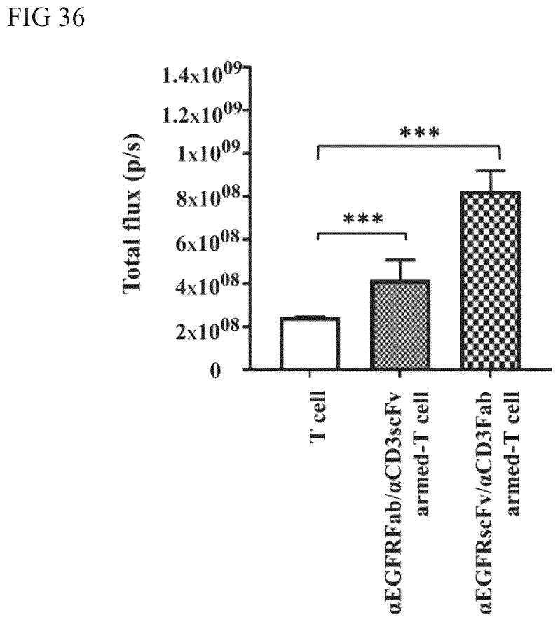

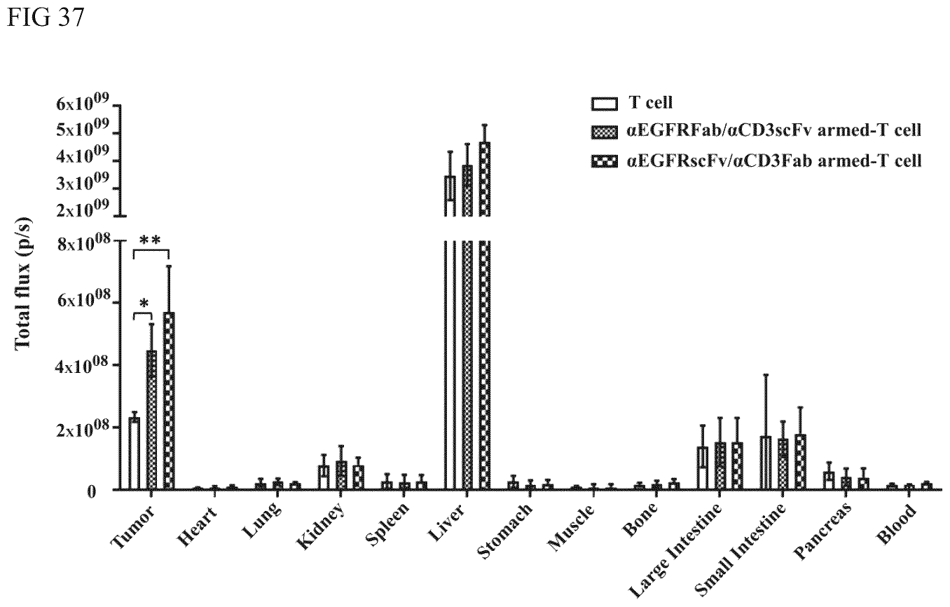

To investigate in vivo effects of the anti-EGFR/anti-CD3 BsAb-armed T cells, NIR797 labeled T cells (10⁷ cells) armed with anti-EGFR Fab/anti-CD3 scFv BsAb, anti-EGFR scFv/anti-CD3 Fab BsAb, or OKT3 antibody were intravenously injected into SCID mice bearing EGFR⁺ colon tumor (HT29, tumor size: about 100 mm³). The animals were sacrificed after 24 hours, and the tumor per se and organs, including heart, lung, kidney, spleen, liver, stomach, muscle, bone, large intestine, small intestine, pancreas, and blood, were harvested and analyzed by IVIS imaging system, respectively. Results are depicted in FIGS. 36 and 37.

The fluorescent intensity in animals treated with the anti-EGFR/anti-CD3 BsAb-armed T cells (i.e., the anti-EGFR Fab/anti-CD3 scFv BsAb-armed T cells or the anti-EGFR scFv/anti-CD3 Fab BsAb-armed T cells) was found mainly concentrated on the tumor site, as compared to that treated with OKT3-armed T cells (FIG. 36). The result confirmed that the T cells modified with the present anti-EGFR/anti-CD3 BsAbs were indeed being targeted to the tumor, thereby resulted in an enhanced fluorescent intensity at the tumor site.

Further, the anti-EGFR/anti-CD3 BsAb-armed T cells were found to be concentrated in the EGFR⁺ tumor per se, while the in vivo distribution pattern of these armed T cells was similar to that of a normal healthy animal (FIG. 37).

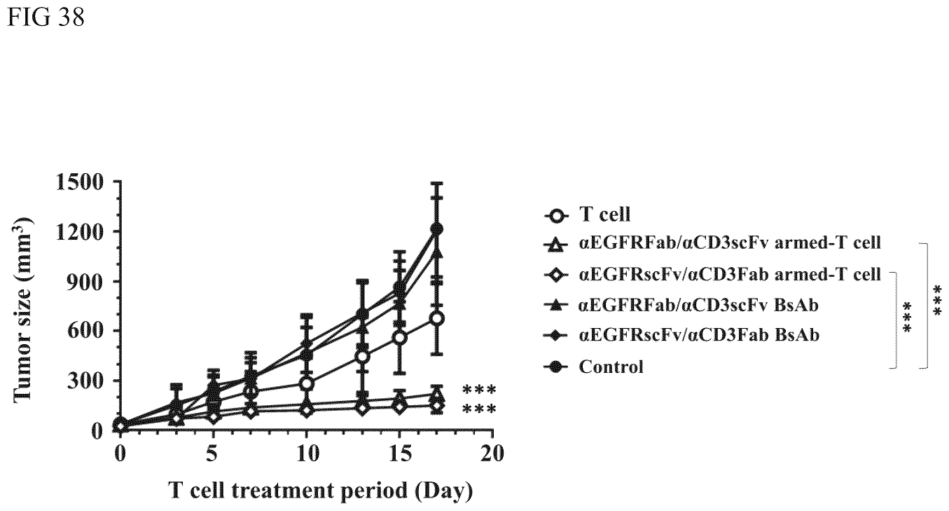

In addition, the tumor size (FIG. 38) and its weight were suppressed significantly when treated with the anti-EGFR/anti-CD3 BsAb-armed T cells. There was no obvious difference in body weight among different groups of mice (data not shown).

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of producing a tumor antigen-specific T cell from peripheral blood mononuclear cells (PBMCs), comprising:
culturing the PBMCs with a bi-specific antibody (BsAb) in a culture medium so as to produce the tumor antigen-specific T cell from the PBMCs,
wherein the BsAb comprises a tumor antigen binding site and a CD3 binding site, wherein
the tumor antigen binding site comprises an anti-tumor antigen scFv 100% identical to SEQ ID NO: 77; and the CD3 binding site comprises an anti-CD3 VL-Ck domain 100% identical to SEQ ID NO: 67, and an anti-CD3 VH-CH1 domain 100% identical to SEQ ID NO: 68; or
the tumor antigen binding site comprises an anti-tumor antigen VL-Ck domain 100% identical to SEQ ID NO: 75, and an anti-tumor antigen VH-CH1 domain 100% identical to SEQ ID NO: 76; the CD3 binding site comprises an anti-CD3 scFv 100% identical to SEQ ID NO: 66.

2. The method of claim 1, wherein the culture medium comprises IL-2 and does not comprise TGF-β, and the tumor antigen-specific T cell is a CD3⁺ and CD8⁺ T cell.

3. The method of claim 1, wherein the PBMCs are isolated from a human subject.

4. A CD3⁺ and CD8⁺ T cell produced by the method of claim 2.

5. A method of treating a subject afflicted with a cancer comprising administering to the subject an effective amount of the CD3⁺ and CD8⁺ T cell of claim 4 so as to inhibit the growth of the cancer.

6. The method of claim 5, wherein the subject is a human.

7. The method of claim 5, wherein the cancer is EGFR⁺ cancer.

8. The method of claim 7, wherein the cancer is bladder cancer, biliary cancer, bone cancer, brain tumor, breast cancer, cervical cancer, colorectal cancer, colon cancer, esophageal cancer, epidermal carcinoma, gastric cancer, gastrointestinal stromal tumor (GIST), glioma, hematopoietic tumors of lymphoid lineage, hepatic cancer, non-Hodgkin's lymphoma, Kaposi's sarcoma, leukemia, lung cancer, lymphoma, intestinal cancer, melanoma, myeloid leukemia, pancreatic cancer, prostate cancer, retinoblastoma, ovary cancer, renal cell carcinoma, spleen cancer, squamous cell carcinoma, thyroid cancer, or thyroid follicular cancer.


SEQUENCE LISTING (143 sequences)

SEQ ID NO: 1 - DNA, 63 bp, synthetic construct
  misc_feature 1..63: note = Synthetic
  source 1..63: mol_type = other DNA
atggagacagacacactcctgctatgggtactgctgctctgggttccaggttccactggtgac

SEQ ID NO: 2 - DNA, 645 bp, synthetic construct
  misc_feature 1..645: note = Synthetic
  source 1..645: mol_type = other DNA
gacatcgtgatgacccagagccccagcagcctgagcgccagcgtgggcgacagagtgaccatcacctgcaaggccagccaggacgtgggcaccgccgtggactggtaccagcagaagcccggcaaggcccccaagctgctgatctactgggccagcaccagacacaccggcgtgcccgacagattcaccggcagcggcagcggcaccgacttcaccctgaccatcagcagcctgcagcccgaggacttcgccgactacttctgccagcagtacaacagctaccccctgaccttcggcggcggcaccaagctggagatcaagagaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 3 - DNA, 585 bp, synthetic construct
  misc_feature 1..585: note = Synthetic
  source 1..585: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 4 - DNA, 660 bp, synthetic construct
  misc_feature 1..660: note = Synthetic
  source 1..660: mol_type = other DNA
gaggtgcagctggtgcagagcggcgccgaggtgaagaagcccggcgccagcgtgaagatcagctgcaagaccagcggctacaccttcaccgagtacaccatccactgggtgaagcaggccagcggcaagggcctggagtggatcggcaacatcaaccccaacaacggcggcaccacctacaaccagaagttcgaggacagagccaccctgaccgtggacaagagcaccagcaccgcctacatggagctgagcagcctgagaagcgaggacaccgccgtgtactactgcgccgccggctggaacttcgactactggggccagggcaccaccgtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 5 - DNA, 774 bp, synthetic construct
  misc_feature 1..774: note = Synthetic
  source 1..774: mol_type = other DNA
ggtggcggctcaggtggaggcgacatcaagctgcagcagagcggcgccgagctggccagacccggcgccagcgtgaagatgagctgcaagaccagcggctacaccttcaccagatacaccatgcactgggtgaagcagagacccggccagggcctggagtggatcggctacatcaaccccagcagaggctacaccaactacaaccagaagttcaaggacaaggccaccctgaccaccgacaagagcagcagcaccgcctacatgcagctgagcagcctgaccagcgaggacagcgccgtgtactactgcgccagatactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagagccagcagcagcgtgagctacatgaactggtaccagcagaagagcggcaccagccccaagagatggatctacgacaccagcaaggtggccagcggcgtgccctacagattcagcggcagcggcagcggcaccagctacagcctgaccatcagcagcatggaggccgaggacgccgccacctactactgccagcagtggagcagcaaccccctgaccttcggcgctggcaccaagctggagctgaagagacaccaccaccaccaccactga

SEQ ID NO: 6 - DNA, 642 bp, synthetic construct
  misc_feature 1..642: note = Synthetic
  source 1..642: mol_type = other DNA
gacatccagctgacccagagccccgccatcatgagcgccagccccggcgagaaggtgaccatgacctgcagagccagcagcagcgtgagctacatgaactggtaccagcagaagagcggcaccagccccaagagatggatctacgacaccagcaaggtggccagcggcgtgccctacagattcagcggcagcggcagcggcaccagctacagcctgaccatcagcagcatggaggccgaggacgccgccacctactactgccagcagtggagcagcaaccccctgaccttcggcgctggcaccaagctggagctgaagagaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 7 - DNA, 651 bp, synthetic construct
  misc_feature 1..651: note = Synthetic
  source 1..651: mol_type = other DNA
gacatcaagctgcagcagagcggcgccgagctggccagacccggcgccagcgtgaagatgagctgcaagaccagcggctacaccttcaccagatacaccatgcactgggtgaagcagagacccggccagggcctggagtggatcggctacatcaaccccagcagaggctacaccaactacaaccagaagttcaaggacaaggccaccctgaccaccgacaagagcagcagcaccgcctacatgcagctgagcagcctgaccagcgaggacagcgccgtgtactactgcgccagatactacgacgaccactactgcctggactactggggccagggcaccaccctgaccgtgagcagcgcctccaccaaaggtccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagtaacaccaaggtggacaagacagtt

SEQ ID NO: 8 - DNA, 759 bp, synthetic construct
  misc_feature 1..759: note = Synthetic
  source 1..759: mol_type = other DNA
ggcggcggcagcggcggcggcgaggtgcagctggtgcagagcggcgccgaggtgaagaagcccggcgccagcgtgaagatcagctgcaagaccagcggctacaccttcaccgagtacaccatccactgggtgaagcaggccagcggcaagggcctggagtggatcggcaacatcaaccccaacaacggcggcaccacctacaaccagaagttcgaggacagagccaccctgaccgtggacaagagcaccagcaccgcctacatggagctgagcagcctgagaagcgaggacaccgccgtgtactactgcgccgccggctggaacttcgactactggggccagggcaccaccgtgaccgtgagcagcgccggtggaggcggttcaggcggaggtggctctggcggtggcggatcggacatcgtgatgacccagagccccagcagcctgagcgccagcgtgggcgacagagtgaccatcacctgcaaggccagccaggacgtgggcaccgccgtggactggtaccagcagaagcccggcaaggcccccaagctgctgatctactgggccagcaccagacacaccggcgtgcccgacagattcaccggcagcggcagcggcaccgacttcaccctgaccatcagcagcctgcagcccgaggacttcgccgactacttctgccagcagtacaacagctaccccctgaccttcggcggcggcaccaagctggagatcaagagacaccaccaccaccaccactga

SEQ ID NO: 9 - DNA, 717 bp, synthetic construct
  misc_feature 1..717: note = Synthetic
  source 1..717: mol_type = other DNA
gacatcgtgatgacccagagccccagcagcctgagcgccagcgtgggcgacagagtgaccatcacctgcaaggccagccaggacgtgggcaccgccgtggactggtaccagcagaagcccggcaaggcccccaagctgctgatctactgggccagcaccagacacaccggcgtgcccgacagattcaccggcagcggcagcggcaccgacttcaccctgaccatcagcagcctgcagcccgaggacttcgccgactacttctgccagcagtacaacagctaccccctgaccttcggcggcggcaccaagctggagatcaagagaggtggaggcggttcaggcggaggtggctctggcggtggcggatcggaggtgcagctggtgcagagcggcgccgaggtgaagaagcccggcgccagcgtgaagatcagctgcaagaccagcggctacaccttcaccgagtacaccatccactgggtgaagcaggccagcggcaagggcctggagtggattggcaacatcaaccccaacaacggcggcaccacctacaaccagaagttcgaggacagagccaccctgaccgtggacaagagcaccagcaccgcctacatggagctgagcagcctgagaagcgaggacaccgccgtgtactactgcgccgctggctggaacttcgactactggggccagggcaccaccgtgaccgtgagcagcgcc

SEQ ID NO: 10 - DNA, 672 bp, synthetic construct
  misc_feature 1..672: note = Synthetic
  source 1..672: mol_type = other DNA
gacatccagctgacccagagcccagcaatcatgtctgccagcccaggagagaaggtgaccatgacatgccgggccagcagcagcgtgagctacatgaactggtatcagcggaagagcggcacctcccctaagagatggatctacgacacatctaaggtggccagcggcgtgccatataggttctccggctctggcagcggcacctcctactctctgacaatcagctccatggaggccgaggatgccgccacctactattgccagcagtggtctagcaatcccctgacctttggcgccggcacaaagctggagctgaagaggaccgtggcagcacctagcgtgttcatctttcccccttccgacgagcagctgaagtccggcacagcctctgtggtgtgcctgctgaacaatttctatccacgcgaggccaaggtgcagtggaaggtggataacgccctgcagtctggcaatagccaggagtccgtgaccgagcaggactctaaggatagcacatactccctgtcctctaccctgacactgagcaaggccgattacgagaagcacaagctgtatgcatgcgaggtgacccaccagggcctgagctccccagtgacaaagtcttttaaccggggcgagtgtggcggcggctcccaccaccaccaccaccactga

SEQ ID NO: 11 - DNA, 1350 bp, synthetic construct
  misc_feature 1..1350: note = Synthetic
  source 1..1350: mol_type = other DNA
gacatcaagctgcagcagagcggagcagagctggccaggcccggagcctctgtgaagatgagctgcaagacctccggctacaccttcacacggtatacaatgcactgggtgaagtacagacctggccagggcctggagtggatcggctatatcaacccatcccgcggctacaccaactataatcagaagtttaaggacaaggccaccctgaccacagataagagctcctctacagcctacatgcagctgagctccctgacctctgaggacagcgccgtgtactattgcgcccggtactatgacgatcactactgtctggattattggggccagggcaccacactgaccgtgtctagcgcctctacaaagggacctagcgtgttcccactggcaccctcctctaagtccacctctggaggaacagccgccctgggatgcctggtgaaggattacttcccagagcccgtgaccgtgtcctggaactctggcgccctgaccagcggagtgcacacatttcctgccgtgctgcagagctccggcctgtactccctgtctagcgtggtgacagtgccatcctctagcctgggcacccagacatatatctgcaacgtgaatcacaagccaagcaataccaaggtcgacaagaaggtggagcccaagtcctgtgataagacccacacatgccccccttgtcctgcaccagagctgctgggaggaccaagcgtgttcctgtttccacccaagcctaaggacaccctgatgatctctcggaccccagaggtgacatgcgtggtggtggacgtgagccacgaggatcccgaggtgaagtttaactggtacgtggatggcgtggaggtgcacaatgccaagaccaagcccagggaggagcagtacaattccacctatcgcgtggtgtctgtgctgacagtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatccagagatgagctgaccaagaaccaggtgtctctgacatgtctggtgaagggcttctatccctctgacatcgccgtggagtgggagagcaatggccagcctgagaacaattacgataccacaccccctgtgctggacagcgatggctccttctttctgtatagcgacctgacagtggataagtccagatggcagcagggcaacgtgtttagctgttccgtgatgcacgaggccctgcacaatcactacacccagaagtctctgagcctgtcccccggcaagtaa

SEQ ID NO: 12 - DNA, 684 bp, synthetic construct
  misc_feature 1..684: note = Synthetic
  source 1..684: mol_type = other DNA
aggatcgtgatgacccagtctcctagctccctgagcgcctccgtgggcgaccgcgtgaccatcacatgcaaggccagccaggatgtgggaaccgcagtggactggtaccaggataagccaggcaaggcccccaagctgctgatctattgggcctccaccaggcacacaggagtgccagacagattcaccggctctggcagcggcacagacttcaccctgacaatctctagcctgcagcccgaggacttcgccgattacttttgccagcagtacaacagctatcctctgaccttcggcggcggcacaaagctggagatcaagaggaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 13 - DNA, 1338 bp, synthetic construct
  misc_feature 1..1338: note = Synthetic
  source 1..1338: mol_type = other DNA
gaggtgcagctggtgcagtccggagcagaggtgaagaagcctggagcctctgtgaagatcagctgcaagacctccggctacaccttcacagagtatacaatccactgggtgaagaaggccagcggcaagggcctggagtggatcggcaacatcaatccaaacaatggcggcaccacatacaaccaggagtttgaggaccgggccaccctgacagtggataagtccacctctacagcctatatggagctgagctccctgagatccgaggacaccgccgtgtactattgcgccgccggctggaatttcgattactggggccagggcaccacagtgaccgtgtctagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 14 - DNA, 645 bp, synthetic construct
  misc_feature 1..645: note = Synthetic
  source 1..645: mol_type = other DNA
gatattctgctgacccagagcccggttattctgagcgttagcccgggtgaacgtgttagctttagctgtcgtgcaagccagagcattggtaccaatattcattggtatcagcagcgtaccaatggtagcccgcgtctgctgattaaatatgcaagcgaaagcattagcggtattccgagccgttttagcggtagcggtagcggtaccgattttaccctgagcattaatagcgttgaaagcgaagatattgcagattattattgtcagcagaataataattggccgaccacctttggtgcaggtaccaaactggaactgaaacgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 15 - DNA, 672 bp, synthetic construct
  misc_feature 1..672: note = Synthetic
  source 1..672: mol_type = other DNA
caggttcagctgaaacagagcggtccgggtctggttcagccgagccagagcctgagcattacctgtaccgttagcggttttagcctgaccaattatggtgttcattgggttcgtcagagcccgggtaaaggtctggaatggctgggtgttatttggagcggtggtaataccgattataataccccgtttaccagccgtctgagcattaataaagataatagcaaaagccaggttttttttaaaatgaatagcctgcagagcaatgataccgcaatttattattgtgcacgtgcactgacctattatgattatgaatttgcatattggggtcagggtaccctggttaccgttagcgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 16 - DNA, 768 bp, synthetic construct
  misc_feature 1..768: note = Synthetic
  source 1..768: mol_type = other DNA
ggcggcggcagcggcggcggccaggttcagctgaaacagagcggtccgggtctggttcagccgagccagagcctgagcattacctgtaccgttagcggttttagcctgaccaattatggtgttcattgggttcgtcagagcccgggtaaaggtctggaatggctgggtgttatttggagcggtggtaataccgattataataccccgtttaccagccgtctgagcattaataaagataatagcaaaagccaggttttttttaaaatgaatagcctgcagagcaatgataccgcaatttattattgtgcacgtgcactgacctattatgattatgaatttgcatattggggtcagggtaccctggttaccgttagcgcaggcggcggcggcagcggcggcggcggcagcggcggcggcggcagcgatattctgctgacccagagcccggttattctgagcgttagcccgggtgaacgtgttagctttagctgtcgtgcaagccagagcattggtaccaatattcattggtatcagcagcgtaccaatggtagcccgcgtctgctgattaaatatgcaagcgaaagcattagcggtattccgagccgttttagcggtagcggtagcggtaccgattttaccctgagcattaatagcgttgaaagcgaagatattgcagattattattgtcagcagaataataattggccgaccacctttggtgcaggtaccaaactggaactgaaacgtcaccaccaccaccaccactga

SEQ ID NO: 17 - DNA, 726 bp, synthetic construct
  misc_feature 1..726: note = Synthetic
  source 1..726: mol_type = other DNA
gatattctgctgacccagagcccggttattctgagcgttagcccgggtgaacgtgttagctttagctgtcgtgcaagccagagcattggtaccaatattcattggtatcagcagcgtaccaatggtagcccgcgtctgctgattaaatatgcaagcgaaagcattagcggtattccgagccgttttagcggtagcggtagcggtaccgattttaccctgagcattaatagcgttgaaagcgaagatattgcagattattattgtcagcagaataataattggccgaccacctttggtgcaggtaccaaactggaactgaaacgtggtggaggcggttcaggcggaggtggctctggcggtggcggatcgcaggttcagctgaaacagagcggtccgggtctggttcagccgagccagagcctgagcattacctgtaccgttagcggttttagcctgaccaattatggtgttcattgggttcgtcagagcccgggtaaaggtctggaatggctgggtgttatttggagcggtggtaataccgattataataccccgtttaccagccgtctgagcattaataaagataatagcaaaagccaggttttttttaaaatgaatagcctgcagagcaatgataccgcaatttattattgtgcacgtgcactgacctattatgattatgaatttgcatattggggtcagggtaccctggttaccgttagcgca

SEQ ID NO: 18 - DNA, 765 bp, synthetic construct
  misc_feature 1..765: note = Synthetic
  source 1..765: mol_type = other DNA
ggcggtggatcagacatcaagctgcagcagagcggcgccgagctggccagacccggcgccagcgtgaagatgagctgcaagaccagcggctacaccttcaccagatacaccatgcactgggtgaagcagagacccggccagggcctggagtggatcggctacatcaaccccagcagaggctacaccaactacaaccagaagttcaaggacaaggccaccctgaccaccgacaagagcagcagcaccgcctacatgcagctgagcagcctgaccagcgaggacagcgccgtgtactactgcgccagatactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagagccagcagcagcgtgagctacatgaactggtaccagcagaagagcggcaccagccccaagagatggatctacgacaccagcaaggtggccagcggcgtgccctacagattcagcggcagcggcagcggcaccagctacagcctgaccatcagcagcatggaggccgaggacgccgccacctactactgccagcagtggagcagcaaccccctgaccttcggcgctggcaccaagctggagctgaagagacaccaccaccaccaccactga

SEQ ID NO: 19 - DNA, 684 bp, synthetic construct
  misc_feature 1..684: note = Synthetic
  source 1..684: mol_type = other DNA
aggattctgctgacccagagcccggttattctgagcgttagcccgggtgaacgtgttagctttagctgtcgtgcaagccagagcattggtaccaatattcattggtatcaggatcgtaccaatggtagcccgcgtctgctgattaaatatgcaagcgaaagcattagcggtattccgagccgttttagcggtagcggtagcggtaccgattttaccctgagcattaatagcgttgaaagcgaagatattgcagattattattgtcagcagaataataattggccgaccacctttggtgcaggtaccaaactggaactgaaacgtaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 20 - DNA, 1350 bp, synthetic construct
  misc_feature 1..1350: note = Synthetic
  source 1..1350: mol_type = other DNA
caggttcagctgaaacagagcggtccgggtctggttcagccgagccagagcctgagcattacctgtaccgttagcggttttagcctgaccaattatggtgttcattgggttcgtaagagcccgggtaaaggtctggaatggctgggtgttatttggagcggtggtaataccgattataataccgagtttaccagccgtctgagcattaataaagataatagcaaaagccaggttttttttaaaatgaatagcctgcagagcaatgataccgcaatttattattgtgcacgtgcactgacctattatgattatgaatttgcatattggggtcagggtaccctggttaccgttagcgcagcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 21 - DNA, 645 bp, synthetic construct
  misc_feature 1..645: note = Synthetic
  source 1..645: mol_type = other DNA
gatatccagatgacccagagccctagctccctgagcgcctccgtgggcgacagggtgaccatcacatgtagggcatcccaggacgtgagcaccgcagtggcctggtatcagcagaagcccggcaaggcccctaagctgctgatctactctgccagcttcctgtatagcggcgtgccttccagattttccggatctggaagcggaaccgacttcaccctgacaatctctagcctgcagccagaggattttgccacatactattgccagcagtacctgtatcacccagcaaccttcggacagggcacaaaagtggaaatcaagcggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 22 - DNA, 669 bp, synthetic construct
  misc_feature 1..669: note = Synthetic
  source 1..669: mol_type = other DNA
gaggtgcagctggtggaaagcgggggaggactggtgcagcctggggggtcactgagactgagttgcgccgcctccggcttcaccttttccgactcttggatccactgggtgaggcaggcaccaggcaagggcctggagtgggtggcctggatctctccctacggcggcagcacctactatgccgactccgtgaagggccggtttacaatctctgccgataccagcaagaacacagcctatctgcagatgaattctctgagagccgaggacaccgccgtgtactattgcgcacggagacactggccaggaggattcgattactggggacagggcaccctggtgacagtgtccgccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 23 - DNA, 774 bp, synthetic construct
  misc_feature 1..774: note = Synthetic
  source 1..774: mol_type = other DNA
gggggcgggagcgggggcggcgaggtgcagctggtggaaagcgggggaggactggtgcagcctggggggtcactgagactgagttgcgccgcctccggcttcaccttttccgactcttggatccactgggtgaggcaggcaccaggcaagggcctggagtgggtggcctggatctctccctacggcggcagcacctactatgccgactccgtgaagggccggtttacaatctctgccgataccagcaagaacacagcctatctgcagatgaattctctgagagccgaggacaccgccgtgtactattgcgcacggagacactggccaggaggattcgattactggggacagggcaccctggtgacagtgtccgccgtggagggaggatccggaggatctggaggaagcggcggctccggcggcgtggacgatatccagatgacccagagccctagctccctgagcgcctccgtgggcgacagggtgaccatcacatgtagggcatcccaggacgtgagcaccgcagtggcctggtatcagcagaagcccggcaaggcccctaagctgctgatctactctgccagcttcctgtatagcggcgtgccttccagattttccggatctggaagcggaaccgacttcaccctgacaatctctagcctgcagccagaggattttgccacatactattgccagcagtacctgtatcacccagcaaccttcggacagggcacaaaagtggaaatcaagcggcatcatcatcatcaccattaa

SEQ ID NO: 24 - DNA, 723 bp, synthetic construct
  misc_feature 1..723: note = Synthetic
  source 1..723: mol_type = other DNA
gatatccagatgacccagagccctagctccctgagcgcctccgtgggcgacagggtgaccatcacatgtagggcatcccaggacgtgagcaccgcagtggcctggtatcagcagaagcccggcaaggcccctaagctgctgatctactctgccagcttcctgtatagcggcgtgccttccagattttccggatctggaagcggaaccgacttcaccctgacaatctctagcctgcagccagaggattttgccacatactattgccagcagtacctgtatcacccagcaaccttcggacagggcacaaaagtggaaatcaagcggggtggaggcggttcaggcggaggtggctctggcggtggcggatcggaggtgcagctggtggaaagcgggggaggactggtgcagcctggggggtcactgagactgagttgcgccgcctccggcttcaccttttccgactcttggatccactgggtgaggcaggcaccaggcaagggcctggagtgggtggcctggatctctccctacggcggcagcacctactatgccgactccgtgaagggccggtttacaatctctgccgataccagcaagaacacagcctatctgcagatgaattctctgagagccgaggacaccgccgtgtactattgcgcacggagacactggccaggaggattcgattactggggacagggcaccctggtgacagtgtccgcc

SEQ ID NO: 25 - DNA, 684 bp, synthetic construct
  misc_feature 1..684: note = Synthetic
  source 1..684: mol_type = other DNA
aggatccagatgacccagagccctagctccctgagcgcctccgtgggcgacagggtgaccatcacatgtagggcatcccaggacgtgagcaccgcagtggcctggtatcaggataagcccggcaaggcccctaagctgctgatctactctgccagcttcctgtatagcggcgtgccttccagattttccggatctggaagcggaaccgacttcaccctgacaatctctagcctgcagccagaggattttgccacatactattgccagcagtacctgtatcacccagcaaccttcggacagggcacaaaagtggaaatcaagcggaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 26 - DNA, 1347 bp, synthetic construct
  misc_feature 1..1347: note = Synthetic
  source 1..1347: mol_type = other DNA
gaggtgcagctggtggaaagcgggggaggactggtgcagcctggggggtcactgagactgagttgcgccgcctccggcttcaccttttccgactcttggatccactgggtgaggaaggcaccaggcaagggcctggagtgggtggcctggatctctccctacggcggcagcacctactatgccgactccgtgaagggcgagtttacaatctctgccgataccagcaagaacacagcctatctgcagatgaattctctgagagccgaggacaccgccgtgtactattgcgcacggagacactggccaggaggattcgattactggggacagggcaccctggtgacagtgtccgccgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 27 - DNA, 645 bp, synthetic construct
  misc_feature 1..645: note = Synthetic
  source 1..645: mol_type = other DNA
gacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagagaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 28 - DNA, 675 bp, synthetic construct
  misc_feature 1..675: note = Synthetic
  source 1..675: mol_type = other DNA
gaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 29 - DNA, 759 bp, synthetic construct; identical to SEQ ID NO: 8
  misc_feature 1..759: note = Synthetic
  source 1..759: mol_type = other DNA

SEQ ID NO: 30 - DNA, 729 bp, synthetic construct
  misc_feature 1..729: note = Synthetic
  source 1..729: mol_type = other DNA
gacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagagaggtggaggcggttcaggcggaggtggctctggcggtggcggatcggaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagc

SEQ ID NO: 31 - DNA, 684 bp, synthetic construct
  misc_feature 1..684: note = Synthetic
  source 1..684: mol_type = other DNA
aggatccagatgacccagagccccagcagcctgagcgccagcgtgggcgacagagtgaccatcacctgcagagccagccaggacgtgaacaccgccgtggcctggtaccaggataagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagagaaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 32 - DNA, 1353 bp, synthetic construct
  misc_feature 1..1353: note = Synthetic
  source 1..1353: mol_type = other DNA
gaggtgcagctggtggagagcggcggcggcctggtgcagcccggcggcagcctgagactgagctgcgccgccagcggcttcaacatcaaggacacctacatccactgggtgagaaaggcccccggcaagggcctggagtgggtggccagaatctaccccaccaacggctacaccagatacgccgacagcgtgaagggcaggttcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 33 - DNA, 660 bp, synthetic construct
  misc_feature 1..660: note = Synthetic
  source 1..660: mol_type = other DNA
gacattgtgatgacccagtcccctgacagcctggctgtgagcctgggagagagggccaccatcaactgcaagagcagccagagcctgctgtacagcagaaaccagaaaaactacctggcctggtaccagcagaaacccggccagcctcccaagctgctgatcttctgggccagcaccagagagagcggcgtgcctgacagattcagcggctccggcttcggcaccgacttcaccctgaccatcagcagcctccaggccgaggatgtcgccgtgtattactgccagcagtacttctcctatcccctcaccttcggccaaggcaccaaggtggagatcaagactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 34 - DNA, 687 bp, synthetic construct
  misc_feature 1..687: note = Synthetic
  source 1..687: mol_type = other DNA
caagtgcagctggtgcagtccggcgctgaggtgaagaagcccggagcctccgtgaaggtgtcctgcaagacctccaggtacaccttcacagagtacaccatccactgggtgagacaggctcccggccagaggctggagtggattggcggcattaaccccaacaacggcatccccaactacaaccagaagttcaagggcagggtgaccatcacagttgatacatccgccagcacagcctatatggaactgagctccctgaggagcgaggacacagccgtgtactactgcgccaggaggaggattgcctacggctatgatgagggccatgccatggactactggggacagggcaccctggtgacagtctccagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 35 - DNA, 807 bp, synthetic construct
  misc_feature 1..807: note = Synthetic
  source 1..807: mol_type = other DNA
ggcggcggcagcggcggcggccaagtgcagctggtgcagtccggcgctgaggtgaagaagcccggagcctccgtgaaggtgtcctgcaagacctccaggtacaccttcacagagtacaccatccactgggtgagacaggctcccggccagaggctggagtggattggcggcattaaccccaacaacggcatccccaactacaaccagaagttcaagggcagggtgaccatcacagttgatacatccgccagcacagcctatatggaactgagctccctgaggagcgaggacacagccgtgtactactgcgccaggaggaggattgcctacggctatgatgagggccatgccatggactactggggacagggcaccctggtgacagtctccagcggcagcaccagcggatccggaaagcctggcagcggagagggaagcaccaagggcgacattgtgatgacccagtcccctgacagcctggctgtgagcctgggagagagggccaccatcaactgcaagagcagccagagcctgctgtacagcagaaaccagaaaaactacctggcctggtaccagcagaaacccggccagcctcccaagctgctgatcttctgggccagcaccagagagagcggcgtgcctgacagattcagcggctccggcttcggcaccgacttcaccctgaccatcagcagcctccaggccgaggatgtcgccgtgtattactgccagcagtacttctcctatcccctcaccttcggccaaggcaccaaggtggagatcaagcaccaccaccaccaccactga

SEQ ID NO: 36 - DNA, 756 bp, synthetic construct
  misc_feature 1..756: note = Synthetic
  source 1..756: mol_type = other DNA
gacattgtgatgacccagtcccctgacagcctggctgtgagcctgggagagagggccaccatcaactgcaagagcagccagagcctgctgtacagcagaaaccagaaaaactacctggcctggtaccagcagaaacccggccagcctcccaagctgctgatcttctgggccagcaccagagagagcggcgtgcctgacagattcagcggctccggcttcggcaccgacttcaccctgaccatcagcagcctccaggccgaggatgtcgccgtgtattactgccagcagtacttctcctatcccctcaccttcggccaaggcaccaaggtggagatcaagggtggaggcggttcaggcggaggtggctctggcggtggcggatcgcaagtgcagctggtgcagtccggcgctgaggtgaagaagcccggagcctccgtgaaggtgtcctgcaagacctccaggtacaccttcacagagtacaccatccactgggtgagacaggctcccggccagaggctggagtggattggcggcattaaccccaacaacggcatccccaactacaaccagaagttcaagggcagggtgaccatcacagttgatacatccgccagcacagcctatatggaactgagctccctgaggagcgaggacacagccgtgtactactgcgccaggaggaggattgcctacggctatgatgagggccatgccatggactactggggacagggcaccctggtgacagtctccagc

SEQ ID NO: 37 - DNA, 699 bp, synthetic construct
  misc_feature 1..699: note = Synthetic
  source 1..699: mol_type = other DNA
aggattgtgatgacccagtcccctgacagcctggctgtgagcctgggagagagggccaccatcaactgcaagagcagccagagcctgctgtacagcagaaaccagaaaaactacctggcctggtaccaggataaacccggccagcctcccaagctgctgatcttctgggccagcaccagagagagcggcgtgcctgacagattcagcggctccggcttcggcaccgacttcaccctgaccatcagcagcctccaggccgaggatgtcgccgtgtattactgccagcagtacttctcctatcccctcaccttcggccaaggcaccaaggtggagatcaagaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 38 - DNA, 1365 bp, synthetic construct
  misc_feature 1..1365: note = Synthetic
  source 1..1365: mol_type = other DNA
caagtgcagctggtgcagtccggcgctgaggtgaagaagcccggagcctccgtgaaggtgtcctgcaagacctccaggtacaccttcacagagtacaccatccactgggtgagaaaggctcccggccagaggctggagtggattggcggcattaaccccaacaacggcatccccaactacaaccagaggttcaagggcagggtgaccatcacagttgatacatccgccagcacagcctatatggaactgagctccctgaggagcgaggacacagccgtgtactactgcgccaggaggaggattgcctacggctatgatgagggccatgccatggactactggggacagggcaccctggtgacagtctccagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 39 - DNA, 657 bp, synthetic construct
  misc_feature 1..657: note = Synthetic
  source 1..657: mol_type = other DNA
gatattcagatgacccagagcccttcaagcctgtccgccagcgtcggggatagagtgaccattacttgccggagcaccaagtccctgctgcactccaacggcatcacatacctgtattggtaccagcagaagcccggcaaggcccctaagctgctgatctatcagatgtccaatctggcctctggcgtgccaagcagattcagctcctctggcagcggcaccgactttaccctgacaatcagctccctgcagcccgaggatttcgccacatactattgcgcccagaacctggagatccctaggacctttggccagggcacaaaggtggagctgaagactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 40 - DNA, 663 bp, synthetic construct
  misc_feature 1..663: note = Synthetic
  source 1..663: mol_type = other DNA
gaggtgcagctggtgcagtccggacctggcctggtgcagccaggaggctccgtgaggatctcttgtgccgccagcggctataccttcacaaactacggcatgaattgggtgaagcaggcacctggcaagggcctggagtggatgggctggatcaacacctatacaggcgagtccacctacgccgactcttttaagggcagattcaccttttctctggatacatccgcctctgccgcctacctgcagatcaatagcctgcgggccgaggacacagccgtgtactattgtgccagattcgccatcaaaggggactactggggacagggcacactgctgaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 41 - DNA, 771 bp, synthetic construct
  misc_feature 1..771: note = Synthetic
  source 1..771: mol_type = other DNA
ggcggcggcagcggcggcggcgaggtgcagctggtgcagtccggacctggcctggtgcagccaggaggctccgtgaggatctcttgtgccgccagcggctataccttcacaaactacggcatgaattgggtgaagcaggcacctggcaagggcctggagtggatgggctggatcaacacctatacaggcgagtccacctacgccgactcttttaagggcagattcaccttttctctggatacatccgcctctgccgcctacctgcagatcaatagcctgcgggccgaggacacagccgtgtactattgtgccagattcgccatcaaaggggactactggggacagggcacactgctgaccgtctcaagcggtggaggcggttcaggcggaggtggctctggcggtggcggatcggatattcagatgacccagagcccttcaagcctgtccgccagcgtcggggatagagtgaccattacttgccggagcaccaagtccctgctgcactccaacggcatcacatacctgtattggtaccagcagaagcccggcaaggcccctaagctgctgatctatcagatgtccaatctggcctctggcgtgccaagcagattcagctcctctggcagcggcaccgactttaccctgacaatcagctccctgcagcccgaggatttcgccacatactattgcgcccagaacctggagatccctaggacctttggccagggcacaaaggtggagctgaagcaccaccaccaccaccactga

SEQ ID NO: 42 - DNA, 762 bp, synthetic construct
  misc_feature 1..762: note = Synthetic
  source 1..762: mol_type = other DNA
gatattcagatgacccagagcccttcaagcctgtccgccagcgtcggggatagagtgaccattacttgccggagcaccaagtccctgctgcactccaacggcatcacatacctgtattggtaccagcagaagcccggcaaggcccctaagctgctgatctatcagatgtccaatctggcctctggcgtgccaagcagattcagctcctctggcagcggcaccgactttaccctgacaatcagctccctgcagcccgaggatttcgccacatactattgcgcccagaacctggagatccctaggacctttggccagggcacaaaggtggagctgaagcgcgccaccccctctcacaacagccaccaggtgccaagcgccggaggaccaaccgccaattccggcacatctggaagcgaggtgcagctggtgcagtccggacctggcctggtgcagccaggaggctccgtgaggatctcttgtgccgccagcggctataccttcacaaactacggcatgaattgggtgaagcaggcacctggcaagggcctggagtggatgggctggatcaacacctatacaggcgagtccacctacgccgactcttttaagggcagattcaccttttctctggatacatccgcctctgccgcctacctgcagatcaatagcctgcgggccgaggacacagccgtgtactattgtgccagattcgccatcaaaggggactactggggacagggcacactgctgaccgtctcaagc

SEQ ID NO: 43 - DNA, 696 bp, synthetic construct
  misc_feature 1..696: note = Synthetic
  source 1..696: mol_type = other DNA
aggattcagatgacccagagcccttcaagcctgtccgccagcgtcggggatagagtgaccattacttgccggagcaccaagtccctgctgcactccaacggcatcacatacctgtattggtaccaggataagcccggcaaggcccctaagctgctgatctatcagatgtccaatctggcctctggcgtgccaagcagattcagctcctctggcagcggcaccgactttaccctgacaatcagctccctgcagcccgaggatttcgccacatactattgcgcccagaacctggagatccctaggacctttggccagggcacaaaggtggagctgaagaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 44 - DNA, 1341 bp, synthetic construct
  misc_feature 1..1341: note = Synthetic
  source 1..1341: mol_type = other DNA
gaggtgcagctggtgcagtccggacctggcctggtgcagccaggaggctccgtgaggatctcttgtgccgccagcggctataccttcacaaactacggcatgaattgggtgaagaaggcacctggcaagggcctggagtggatgggctggatcaacacctatacaggcgagtccacctacgccgactcttttaagggcgagttcaccttttctctggatacatccgcctctgccgcctacctgcagatcaatagcctgcgggccgaggacacagccgtgtactattgtgccagattcgccatcaaaggggactactggggacagggcacactgctgaccgtctcaagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 45 - DNA, 642 bp, synthetic construct
  misc_feature 1..642: note = Synthetic
  source 1..642: mol_type = other DNA
gagctgcagatgacccagtcccctagctctttaagcgcttccgtgggcgacagagtgaccatcacatgtcgtacctcccagagcatcagctcctatttaaactggtaccagcagaagcccggacagccccccaagctgctgatctactgggcttccactcgtgaaagcggcgtgcccgatcgtttttccggcagcggcagcggcaccgactttactttaaccatctcctctttacagcccgaggatagcgccacctactactgccagcagagctacgacatcccctacacattcggccaaggtaccaagctggaaatcaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 46 - DNA, 696 bp, synthetic construct
  misc_feature 1..696: note = Synthetic
  source 1..696: mol_type = other DNA
gaagtgcagctgctggagagcggaggcggagtggtgcaacccggtcgttctttaaggctgagctgcgctgccagcggcttcaccttcagcagctatggcatgcactgggtgagacaagctcccggaaaaggtttagagtgggtggccgtgatctcctacgacggcagcaacaagtactacgccgacagcgtgaagggtcgtttcaccatctctcgtgacaactccaagaacactttatacctccagatgaactctttaagggccgaggacaccgccgtgtactactgtgccaaggacatgggctggggcagcggatggaggccctactactactacggcatggacgtgtggggccaaggtacaaccgtgaccgtgtccagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 47 - DNA, 789 bp, synthetic construct
  misc_feature 1..789: note = Synthetic
  source 1..789: mol_type = other DNA
ggcggcggcagcggcggcggcgaagtgcagctgctggagagcggaggcggagtggtgcaacccggtcgttctttaaggctgagctgcgctgccagcggcttcaccttcagcagctatggcatgcactgggtgagacaagctcccggaaaaggtttagagtgggtggccgtgatctcctacgacggcagcaacaagtactacgccgacagcgtgaagggtcgtttcaccatctctcgtgacaactccaagaacactttatacctccagatgaactctttaagggccgaggacaccgccgtgtactactgtgccaaggacatgggctggggcagcggatggaggccctactactactacggcatggacgtgtggggccaaggtacaaccgtgaccgtgtccagcggtggaggcggttcaggcggaggtggctctggcggtggcggatcggagctgcagatgacccagtcccctagctctttaagcgcttccgtgggcgacagagtgaccatcacatgtcgtacctcccagagcatcagctcctatttaaactggtaccagcagaagcccggacagccccccaagctgctgatctactgggcttccactcgtgaaagcggcgtgcccgatcgtttttccggcagcggcagcggcaccgactttactttaaccatctcctctttacagcccgaggatagcgccacctactactgccagcagagctacgacatcccctacacattcggccaaggtaccaagctggaaatcaaacaccaccaccaccaccactga

SEQ ID NO: 48 - DNA, 747 bp, synthetic construct
  misc_feature 1..747: note = Synthetic
  source 1..747: mol_type = other DNA
gagctgcagatgacccagtcccctagctctttaagcgcttccgtgggcgacagagtgaccatcacatgtcgtacctcccagagcatcagctcctatttaaactggtaccagcagaagcccggacagccccccaagctgctgatctactgggcttccactcgtgaaagcggcgtgcccgatcgtttttccggcagcggcagcggcaccgactttactttaaccatctcctctttacagcccgaggatagcgccacctactactgccagcagagctacgacatcccctacacattcggccaaggtaccaagctggaaatcaaaggcggcggcggcagcggcggcggcggctccggcggaggaggctccgaagtgcagctgctggagagcggaggcggagtggtgcaacccggtcgttctttaaggctgagctgcgctgccagcggcttcaccttcagcagctatggcatgcactgggtgagacaagctcccggaaaaggtttagagtgggtggccgtgatctcctacgacggcagcaacaagtactacgccgacagcgtgaagggtcgtttcaccatctctcgtgacaactccaagaacactttatacctccagatgaactctttaagggccgaggacaccgccgtgtactactgtgccaaggacatgggctggggcagcggatggaggccctactactactacggcatggacgtgtggggccaaggtacaaccgtgaccgtgtccagc

SEQ ID NO: 49 - DNA, 681 bp, synthetic construct
  misc_feature 1..681: note = Synthetic
  source 1..681: mol_type = other DNA
aggctgcagatgacccagtcccctagctctttaagcgcttccgtgggcgacagagtgaccatcacatgtcgtacctcccagagcatcagctcctatttaaactggtaccaggataagcccggacagccccccaagctgctgatctactgggcttccactcgtgaaagcggcgtgcccgatcgtttttccggcagcggcagcggcaccgactttactttaaccatctcctctttacagcccgaggatagcgccacctactactgccagcagagctacgacatcccctacacattcggccaaggtaccaagctggaaatcaaaaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 50 - DNA, 1374 bp, synthetic construct
  misc_feature 1..1374: note = Synthetic
  source 1..1374: mol_type = other DNA
gaagtgcagctgctggagagcggaggcggagtggtgcaacccggtcgttctttaaggctgagctgcgctgccagcggcttcaccttcagcagctatggcatgcactgggtgagaaaggctcccggaaaaggtttagagtgggtggccgtgatctcctacgacggcagcaacaagtactacgccgacagcgtgaagggtgagttcaccatctctcgtgacaactccaagaacactttatacctccagatgaactctttaagggccgaggacaccgccgtgtactactgtgccaaggacatgggctggggcagcggatggaggccctactactactacggcatggacgtgtggggccaaggtacaaccgtgaccgtgtccagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 51 - DNA, 648 bp, synthetic construct
  misc_feature 1..648: note = Synthetic
  source 1..648: mol_type = other DNA
gatattcagatgacccagagcccgagcagcctgagcgcaagcgttggtgatcgtgttaccattacctgtaaagcaagccagaccgttagcgcaaatgttgcatggtatcagcagaaaccgggtaaagcaccgaaactgctgatttatctggcaagctatcgttatcgtggtgttccgagccgttttagcggtagcggtagcggtaccgattttaccctgaccattagcagcctgcagccggaagattttgcaacctattattgtcatcagtattatacctatccgctgtttacctttggtcagggtaccaaactggaaattaaacgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 52 - DNA, 678 bp, synthetic construct
  misc_feature 1..678: note = Synthetic
  source 1..678: mol_type = other DNA
caggttcagctggttcagagcggtagcgaactgaaaaaaccgggtgcaagcgttaaagttagctgtaaagcaagcggttatacctttaccgaatatggtatgaatgtttggcgtcaggcaccgggtcagggtctggaatggatgggttggattaataccaaaagcggtgaagcaacctatgttgaagaatttaaaggtcgttttgtttttagcctggataccagcgttagcaccgcatatctgcagattagcagcctgaaagcagaagataccgcagtttattattgtgcacgttgggatttttatgattatgttgatgaagcaatgtattggggtcagggtaccaccgttaccgttagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 53 - DNA, 777 bp, synthetic construct
  misc_feature 1..777: note = Synthetic
  source 1..777: mol_type = other DNA
ggcggcggcagcggcggcggccaggttcagctggttcagagcggtagcgaactgaaaaaaccgggtgcaagcgttaaagttagctgtaaagcaagcggttatacctttaccgaatatggtatgaatgtttggcgtcaggcaccgggtcagggtctggaatggatgggttggattaataccaaaagcggtgaagcaacctatgttgaagaatttaaaggtcgttttgtttttagcctggataccagcgttagcaccgcatatctgcagattagcagcctgaaagcagaagataccgcagtttattattgtgcacgttgggatttttatgattatgttgatgaagcaatgtattggggtcagggtaccaccgttaccgttagcagcggtggaggcggttcaggcggaggtggctctggcggtggcggatcggatattcagatgacccagagcccgagcagcctgagcgcaagcgttggtgatcgtgttaccattacctgtaaagcaagccagaccgttagcgcaaatgttgcatggtatcagcagaaaccgggtaaagcaccgaaactgctgatttatctggcaagctatcgttatcgtggtgttccgagccgttttagcggtagcggtagcggtaccgattttaccctgaccattagcagcctgcagccggaagattttgcaacctattattgtcatcagtattatacctatccgctgtttacctttggtcagggtaccaaactggaaattaaacgtcaccaccaccaccaccactga

SEQ ID NO: 54 - DNA, 735 bp, synthetic construct
  misc_feature 1..735: note = Synthetic
  source 1..735: mol_type = other DNA
gatattcagatgacccagagcccgagcagcctgagcgcaagcgttggtgatcgtgttaccattacctgtaaagcaagccagaccgttagcgcaaatgttgcatggtatcagcagaaaccgggtaaagcaccgaaactgctgatttatctggcaagctatcgttatcgtggtgttccgagccgttttagcggtagcggtagcggtaccgattttaccctgaccattagcagcctgcagccggaagattttgcaacctattattgtcatcagtattatacctatccgctgtttacctttggtcagggtaccaaactggaaattaaacgtggtggaggcggttcaggcggaggtggctctggcggtggcggatcgcaggttcagctggttcagagcggtagcgaactgaaaaaaccgggtgcaagcgttaaagttagctgtaaagcaagcggttatacctttaccgaatatggtatgaatgtttggcgtcaggcaccgggtcagggtctggaatggatgggttggattaataccaaaagcggtgaagcaacctatgttgaagaatttaaaggtcgttttgtttttagcctggataccagcgttagcaccgcatatctgcagattagcagcctgaaagcagaagataccgcagtttattattgtgcacgttgggatttttatgattatgttgatgaagcaatgtattggggtcagggtaccaccgttaccgttagcagc

SEQ ID NO: 55 - DNA, 687 bp, synthetic construct
  misc_feature 1..687: note = Synthetic
  source 1..687: mol_type = other DNA
aggattcagatgacccagagcccgagcagcctgagcgcaagcgttggtgatcgtgttaccattacctgtaaagcaagccagaccgttagcgcaaatgttgcatggtatcaggataaaccgggtaaagcaccgaaactgctgatttatctggcaagctatcgttatcgtggtgttccgagccgttttagcggtagcggtagcggtaccgattttaccctgaccattagcagcctgcagccggaagattttgcaacctattattgtcatcagtattatacctatccgctgtttacctttggtcagggtaccaaactggaaattaaacgtaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 56 - DNA, 1356 bp, synthetic construct
  misc_feature 1..1356: note = Synthetic
  source 1..1356: mol_type = other DNA
caggttcagctggttcagagcggtagcgaactgaaaaaaccgggtgcaagcgttaaagttagctgtaaagcaagcggttatacctttaccgaatatggtatgaatgtttggcgtaaggcaccgggtcagggtctggaatggatgggttggattaataccaaaagcggtgaagcaacctatgttgaagaatttaaaggtcgttttgtttttagcctggataccagcgttagcaccgcatatctgcagattagcagcctgaaagcagaagataccgcagtttattattgtgcacgttgggatttttatgattatgttgatgaagcaatgtattggggtcagggtaccaccgttaccgttagcagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 57 - DNA, 654 bp, synthetic construct
  misc_feature 1..654: note = Synthetic
  source 1..654: mol_type = other DNA
gatattcagatgacccagagcccgagcagcctgagcgcgagcgtgggcgatcgcgtgaccattacctgccaggcgagccagagcattagcagcagctatctgagctggtatcagcagaaaccgggcaaagcgccgaaactgctgatttatgcggtgagctatctggcgagcggcgtgccgagccgctttagcggcagcggcagcggcaccgatttcaccctgaccattagcagcctgcagccggaagattttgcgacctattattgccagagcggctattatagcgcgggcgatctgacctttggccagggcaccaaagtggaaattaaacgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 58 - DNA, 660 bp, synthetic construct
  misc_feature 1..660: note = Synthetic
  source 1..660: mol_type = other DNA
gaagtgcagctggtggaaagcggcggcggcctggtgcagccgggcggcagcctgcgcctgagctgcgcggcgagcggctttagcctgagcaactattatattaactgggtgcgccaggcgccgggcaaaggcctggaatgggtggcgattattttttgcgcgggcaacgcgtataacgcgagctgggcgaaaggccgctttaccattagccgcgatgatagcaaaaacaccctgtatctgcagatgaacagcctgcgcgcggaagataccgcggtgtattattgcgcgcgcggctggcaggcgctggtgaactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaa

SEQ ID NO: 59 - DNA, 765 bp, synthetic construct
  misc_feature 1..765: note = Synthetic
  source 1..765: mol_type = other DNA
ggcggcggcagcggcggcggcgaagtgcagctggtggaaagcggcggcggcctggtgcagccgggcggcagcctgcgcctgagctgcgcggcgagcggctttagcctgagcaactattatattaactgggtgcgccaggcgccgggcaaaggcctggaatgggtggcgattattttttgcgcgggcaacgcgtataacgcgagctgggcgaaaggccgctttaccattagccgcgatgatagcaaaaacaccctgtatctgcagatgaacagcctgcgcgcggaagataccgcggtgtattattgcgcgcgcggctggcaggcgctggtgaactggggccagggcaccctggtgaccgtgagcagcggcggcggcggcagcggcggcggcggcagcggcggcggcggcagcgatattcagatgacccagagcccgagcagcctgagcgcgagcgtgggcgatcgcgtgaccattacctgccaggcgagccagagcattagcagcagctatctgagctggtatcagcagaaaccgggcaaagcgccgaaactgctgatttatgcggtgagctatctggcgagcggcgtgccgagccgctttagcggcagcggcagcggcaccgatttcaccctgaccattagcagcctgcagccggaagattttgcgacctattattgccagagcggctattatagcgcgggcgatctgacctttggccagggcaccaaagtggaaattaaacgccaccaccaccaccaccactga

SEQ ID NO: 60 - DNA, 723 bp, synthetic construct
  misc_feature 1..723: note = Synthetic
  source 1..723: mol_type = other DNA
gatattcagatgacccagagcccgagcagcctgagcgcgagcgtgggcgatcgcgtgaccattacctgccaggcgagccagagcattagcagcagctatctgagctggtatcagcagaaaccgggcaaagcgccgaaactgctgatttatgcggtgagctatctggcgagcggcgtgccgagccgctttagcggcagcggcagcggcaccgatttcaccctgaccattagcagcctgcagccggaagattttgcgacctattattgccagagcggctattatagcgcgggcgatctgacctttggccagggcaccaaagtggaaattaaacgcggtggaggcggttcaggcggaggtggctctggcggtggcggatcggaagtgcagctggtggaaagcggcggcggcctggtgcagccgggcggcagcctgcgcctgagctgcgcggcgagcggctttagcctgagcaactattatattaactgggtgcgccaggcgccgggcaaaggcctggaatgggtggcgattattttttgcgcgggcaacgcgtataacgcgagctgggcgaaaggccgctttaccattagccgcgatgatagcaaaaacaccctgtatctgcagatgaacagcctgcgcgcggaagataccgcggtgtattattgcgcgcgcggctggcaggcgctggtgaactggggccagggcaccctggtgaccgtgagcagc

SEQ ID NO: 61 - DNA, 693 bp, synthetic construct
  misc_feature 1..693: note = Synthetic
  source 1..693: mol_type = other DNA
aggattcagatgacccagagcccgagcagcctgagcgcgagcgtgggcgatcgcgtgaccattacctgccaggcgagccagagcattagcagcagctatctgagctggtatcaggataaaccgggcaaagcgccgaaactgctgatttatgcggtgagctatctggcgagcggcgtgccgagccgctttagcggcagcggcagcggcaccgatttcaccctgaccattagcagcctgcagccggaagattttgcgacctattattgccagagcggctattatagcgcgggcgatctgacctttggccagggcaccaaagtggaaattaaacgcaccgtggcagcaccatccgtgttcatctttcccccttctgacgagcagctgaagtctggcacagccagcgtggtgtgctacctgaacaacttctaccccagggaggccaaggtgcagtggaaggtggataacgccctgcagtccggcaattctcaggagagcgtgaccgagcaggactccaaggattctacatacagcctgtggtccaccctgacactgtccaaggccgactacgagaagcacaagctgtatgcatgcgaggtgacccaccagggcctgtcctctcccgtgacaaagagcttcaataggggagagtgtggaggaggatcctacccttatgacgtgccagattatgcctaa

SEQ ID NO: 62 - DNA, 1338 bp, synthetic construct
  misc_feature 1..1338: note = Synthetic
  source 1..1338: mol_type = other DNA
gaagtgcagctggtggaaagcggcggcggcctggtgcagccgggcggcagcctgcgcctgagctgcgcggcgagcggctttagcctgagcaactattatattaactgggtgcgcaaggcgccgggcaaaggcctggaatgggtggcgattattttttgcgcgggcaacgcgtataacgcgagctgggcgaaaggcgagtttaccattagccgcgatgatagcaaaaacaccctgtatctgcagatgaacagcctgcgcgcggaagataccgcggtgtattattgcgcgcgcggctggcaggcgctggtgaactggggccagggcaccctggtgaccgtgagcagcgcctctacaaagggaccaagcgtgtttccactggcaccctcctctaagagcacctccggaggaacagccgccctgggctgtctggtgaaggactatttcccagagcccgtgaccgtgtcctggaactctggcgccctgacctccggagtggcaacaggaccagccgtgctgcagagctccggcctgtacagcctgtctagcgtggtgaccgtgccctcctctagcctgggcacccagacatatatctgcaacgtgaatcacaagccaagcaatacaaaggtcgacaagaaggtggagcccaagtcctgtgataagacccacacatgccccccttgtcctgcaccagagctgctgggaggaccaagcgtgttcctgtttccacccaagcctaaggacaccctgatgatctctaggacccccgaggtgacatgcgtggtggtggacgtgagccacgaggatcctgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgcccgcccctatcgagaagaccatctctaaggccaagggccagcctagggagccacaggtgtacacactgcctccatcccgcaaggagctgaccaagaaccaggtgtctctgacatgtctggtgaagggcttctatccttctgatatcgccgtggagtgggagagcaatggccagccagagaacaattacaagaccacaccccctgtgctgaagagcgacggctccttctttctgtatagcaagctgaccgtggataagtccagatggcagcagggcaacgtgttttcttgtagcgtgatgcacgaggccctgcacaatcactacacacagaagtccctgtctctgagccctggcaagtaa

SEQ ID NO: 63 - protein, 21 aa, synthetic construct
  REGION 1..21: note = Synthetic
METDTLLLWVLLLWVPGSTGD

SEQ ID NO: 64 - protein, 214 aa, synthetic construct
  REGION 1..214: note = Synthetic
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 65 - protein, 220 aa, synthetic construct
  REGION 1..220: note = Synthetic
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 66 - protein, 257 aa, synthetic construct
  REGION 1..257: note = Synthetic
GGGSGGGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRHHHHHH

SEQ ID NO: 67 - protein, 213 aa, synthetic construct
  REGION 1..213: note = Synthetic
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 68 - protein, 217 aa, synthetic construct
  REGION 1..217: note = Synthetic
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 69 - protein, 252 aa, synthetic construct
  REGION 1..252: note = Synthetic
GGGSGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSAGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKRHHHHHH

SEQ ID NO: 70 - protein, 239 aa, synthetic construct
  REGION 1..239: note = Synthetic
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKRGGGGSGGGGSGGGGSEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSA

SEQ ID NO: 71 - protein, 223 aa, synthetic construct
  REGION 1..223: note = Synthetic
DIQLTQSPAIMSASPGEKVTMTCRASSSVSYMNWYQRKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECGGGSHHHHHH

SEQ ID NO: 72 - protein, 449 aa, synthetic construct
  REGION 1..449: note = Synthetic
DIKLQQSGAELARPGASVKMSCKTSGYTFTRYTMHWVKYRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 73 - protein, 227 aa, synthetic construct
  REGION 1..227: note = Synthetic
RIVMTQSPSSLSASVGDRVTITCKASQDVGTAVDWYQDKPGKAPKLLIYWASTRHTGVPDRFTGSGSGTDFTLTISSLQPEDFADYFCQQYNSYPLTFGGGTKLEIKRTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 74 - protein, 445 aa, synthetic construct
  REGION 1..445: note = Synthetic
EVQLVQSGAEVKKPGASVKISCKTSGYTFTEYTIHWVKKASGKGLEWIGNINPNNGGTTYNQEFEDRATLTVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 75 - protein, 214 aa, synthetic construct
  REGION 1..214: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 76 - protein, 224 aa, synthetic construct
  REGION 1..224: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 77 - protein, 255 aa, synthetic construct
  REGION 1..255: note = Synthetic
GGGSGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRHHHHHH

SEQ ID NO: 78 - protein, 242 aa, synthetic construct
  REGION 1..242: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 79 - protein, 254 aa, synthetic construct
  REGION 1..254: note = Synthetic
GGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRHHHHHH

SEQ ID NO: 80 - protein, 227 aa, synthetic construct
  REGION 1..227: note = Synthetic
RILLTQSPVILSVSPGERVSFSCRASQSIGTNIHWYQDRTNGSPRLLIKYASESISGIPSRFSGSGSGTDFTLSINSVESEDIADYYCQQNNNWPTTFGAGTKLELKRTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 81 - protein, 449 aa, synthetic construct
  REGION 1..449: note = Synthetic
QVQLKQSGPGLVQPSQSLSITCTVSGFSLTNYGVHWVRKSPGKGLEWLGVIWSGGNTDYNTEFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 82 - protein, 214 aa, synthetic construct
  REGION 1..214: note = Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 83 - protein, 223 aa, synthetic construct
  REGION 1..223: note = Synthetic
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 84 - protein, 257 aa, synthetic construct
  REGION 1..257: note = Synthetic
GGGSGGGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAVEGGSGGSGGSGGSGGVDDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRHHHHHH

SEQ ID NO: 85 - protein, 241 aa, synthetic construct
  REGION 1..241: note = Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 86 - protein, 227 aa, synthetic construct
  REGION 1..227: note = Synthetic
RIQMTQSPSSLSASVGDRVTITCRASQDVSTAVAWYQDKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 87 - protein, 448 aa, synthetic construct
  REGION 1..448: note = Synthetic
EVQLVESGGGLVQPGGSLRLSCAASGFTFSDSWIHWVRKAPGKGLEWVAWISPYGGSTYYADSVKGEFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 88 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 89 - protein, 225 aa, synthetic construct
  REGION 1..225: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 90 - protein, 256 aa, synthetic construct
  REGION 1..256: note = Synthetic
GGGSGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRHHHHHH

SEQ ID NO: 91 - protein, 243 aa, synthetic construct
  REGION 1..243: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 92 - protein, 227 aa, synthetic construct
  REGION 1..227: note = Synthetic
RIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 93 - protein, 450 aa, synthetic construct
  REGION 1..450: note = Synthetic
EVQLVESGGGLVQPGGSLRLSCAASGFNIKDTYIHWVRKAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 94 - protein, 219 aa, synthetic construct
  REGION 1..219: note = Synthetic
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 95 - protein, 229 aa, synthetic construct
  REGION 1..229: note = Synthetic
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 96 - protein, 268 aa, synthetic construct
  REGION 1..268: note = Synthetic
GGGSGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKGDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKHHHHHH

SEQ ID NO: 97 - protein, 252 aa, synthetic construct
  REGION 1..252: note = Synthetic
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGGSGGGGSGGGGSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 98 - protein, 232 aa, synthetic construct
  REGION 1..232: note = Synthetic
RIVMTQSPDSLAVSLGERATINCKSSQSLLYSRNQKNYLAWYQDKPGQPPKLLIFWASTRESGVPDRFSGSGFGTDFTLTISSLQAEDVAVYYCQQYFSYPLTFGQGTKVEIKTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 99 - protein, 454 aa, synthetic construct
  REGION 1..454: note = Synthetic
QVQLVQSGAEVKKPGASVKVSCKTSRYTFTEYTIHWVRKAPGQRLEWIGGINPNNGIPNYNQRFKGRVTITVDTSASTAYMELSSLRSEDTAVYYCARRRIAYGYDEGHAMDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 100 - protein, 218 aa, synthetic construct
  REGION 1..218: note = Synthetic
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 101 - protein, 221 aa, synthetic construct
  REGION 1..221: note = Synthetic
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 102 - protein, 256 aa, synthetic construct
  REGION 1..256: note = Synthetic
GGGSGGGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKHHHHHH

SEQ ID NO: 103 - protein, 254 aa, synthetic construct
  REGION 1..254: note = Synthetic
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 104 - protein, 231 aa, synthetic construct
  REGION 1..231: note = Synthetic
RIQMTQSPSSLSASVGDRVTITCRSTKSLLHSNGITYLYWYQDKPGKAPKLLIYQMSNLASGVPSRFSSSGSGTDFTLTISSLQPEDFATYYCAQNLEIPRTFGQGTKVELKTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 105 - protein, 446 aa, synthetic construct
  REGION 1..446: note = Synthetic
EVQLVQSGPGLVQPGGSVRISCAASGYTFTNYGMNWVKKAPGKGLEWMGWINTYTGESTYADSFKGEFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 106 - protein, 213 aa, synthetic construct
  REGION 1..213: note = Synthetic
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 107 - protein, 232 aa, synthetic construct
  REGION 1..232: note = Synthetic
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 108 - protein, 262 aa, synthetic construct
  REGION 1..262: note = Synthetic
GGGSGGGEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSGGGGSGGGGSGGGGSELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKHHHHHH

SEQ ID NO: 109 - protein, 249 aa, synthetic construct
  REGION 1..249: note = Synthetic
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKGGGGSGGGGSGGGGSEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 110 - protein, 226 aa, synthetic construct
  REGION 1..226: note = Synthetic
RLQMTQSPSSLSASVGDRVTITCRTSQSISSYLNWYQDKPGQPPKLLIYWASTRESGVPDRFSGSGSGTDFTLTISSLQPEDSATYYCQQSYDIPYTFGQGTKLEIKTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 111 - protein, 457 aa, synthetic construct
  REGION 1..457: note = Synthetic
EVQLLESGGGVVQPGRSLRLSCAASGFTFSSYGMHWVRKAPGKGLEWVAVISYDGSNKYYADSVKGEFTISRDNSKNTLYLQMNSLRAEDTAVYYCAKDMGWGSGWRPYYYYGMDVWGQGTTVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 112 - protein, 215 aa, synthetic construct
  REGION 1..215: note = Synthetic
DIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 113 - protein, 226 aa, synthetic construct
  REGION 1..226: note = Synthetic
QVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 114 - protein, 258 aa, synthetic construct
  REGION 1..258: note = Synthetic
GGGSGGGQVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSGGGGSGGGGSGGGGSDIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIKRHHHHHH

SEQ ID NO: 115 - protein, 245 aa, synthetic construct
  REGION 1..245: note = Synthetic
DIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIKRGGGGSGGGGSGGGGSQVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSS

SEQ ID NO: 116 - protein, 228 aa, synthetic construct
  REGION 1..228: note = Synthetic
RIQMTQSPSSLSASVGDRVTITCKASQTVSANVAWYQDKPGKAPKLLIYLASYRYRGVPSRFSGSGSGTDFTLTISSLQPEDFATYYCHQYYTYPLFTFGQGTKLEIKRTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 117 - protein, 451 aa, synthetic construct
  REGION 1..451: note = Synthetic
QVQLVQSGSELKKPGASVKVSCKASGYTFTEYGMNVWRKAPGQGLEWMGWINTKSGEATYVEEFKGRFVFSLDTSVSTAYLQISSLKAEDTAVYYCARWDFYDYVDEAMYWGQGTTVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 118 - protein, 217 aa, synthetic construct
  REGION 1..217: note = Synthetic
DIQMTQSPSSLSASVGDRVTITCQASQSISSSYLSWYQQKPGKAPKLLIYAVSYLASGVPSRFSGSGSGTDFTLTISSLQPEDFATYYCQSGYYSAGDLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 119 - protein, 220 aa, synthetic construct
  REGION 1..220: note = Synthetic
EVQLVESGGGLVQPGGSLRLSCAASGFSLSNYYINWVRQAPGKGLEWVAIIFCAGNAYNASWAKGRFTISRDDSKNTLYLQMNSLRAEDTAVYYCARGWQALVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 120 - protein, 254 aa, synthetic construct
  REGION 1..254: note = Synthetic
GGGSGGGEVQLVESGGGLVQPGGSLRLSCAASGFSLSNYYINWVRQAPGKGLEWVAIIFCAGNAYNASWAKGRFTISRDDSKNTLYLQMNSLRAEDTAVYYCARGWQALVNWGQGTLVTVSSGGGGSGGGGSGGGGSDIQMTQSPSSLSASVGDRVTITCQASQSISSSYLSWYQQKPGKAPKLLIYAVSYLASGVPSRFSGSGSGTDFTLTISSLQPEDFATYYCQSGYYSAGDLTFGQGTKVEIKRHHHHHH

SEQ ID NO: 121 - protein, 241 aa, synthetic construct
  REGION 1..241: note = Synthetic
DIQMTQSPSSLSASVGDRVTITCQASQSISSSYLSWYQQKPGKAPKLLIYAVSYLASGVPSRFSGSGSGTDFTLTISSLQPEDFATYYCQSGYYSAGDLTFGQGTKVEIKRGGGGSGGGGSGGGGSEVQLVESGGGLVQPGGSLRLSCAASGFSLSNYYINWVRQAPGKGLEWVAIIFCAGNAYNASWAKGRFTISRDDSKNTLYLQMNSLRAEDTAVYYCARGWQALVNWGQGTLVTVSS

SEQ ID NO: 122 - protein, 230 aa, synthetic construct
  REGION 1..230: note = Synthetic
RIQMTQSPSSLSASVGDRVTITCQASQSISSSYLSWYQDKPGKAPKLLIYAVSYLASGVPSRFSGSGSGTDFTLTISSLQPEDFATYYCQSGYYSAGDLTFGQGTKVEIKRTVAAPSVFIFPPSDEQLKSGTASVVCYLNNFYPREAKVQWKVDNALQSGNSQESVTEQDSKDSTYSLWSTLTLSKADYEKHKLYACEVTHQGLSSPVTKSFNRGECGGGSYPYDVPDYA

SEQ ID NO: 123 - protein, 445 aa, synthetic construct
  REGION 1..445: note = Synthetic
EVQLVESGGGLVQPGGSLRLSCAASGFSLSNYYINWVRKAPGKGLEWVAIIFCAGNAYNASWAKGEFTISRDDSKNTLYLQMNSLRAEDTAVYYCARGWQALVNWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 124 - protein, 119 aa, synthetic construct
  REGION 1..119: note = Synthetic
QVQLVQSGAEVKKPGASVKVSCKASGYTFTRYTMHWVRQAPGQGLEWMGYINPSRGYTSYAQKFQGRVTMTTDKSTSTVYMELSSLRSEDTAVYYCARYYDDHYCLDYWGQGTTVTVSS

SEQ ID NO: 125 - protein, 5 aa, synthetic construct
  REGION 1..5: note = Synthetic
RYTMH

SEQ ID NO: 126 - protein, 17 aa, synthetic construct
  REGION 1..17: note = Synthetic
YINPSRGYTSYAQKFQG

SEQ ID NO: 127 - protein, 10 aa, synthetic construct
  REGION 1..10: note = Synthetic
YYDDHYCLDY

SEQ ID NO: 128 - protein, 107 aa, synthetic construct
  REGION 1..107: note = Synthetic
DIQMTQSPSSLSASVGDRVTITCRASSSVSYMNWYQQKPGKAPKLLIYDTSKVASGVPSRFSGSGSGTDYTLTISSLQPEDFATYYCQQWSSNPLTFGGGTKVEIKR

SEQ ID NO: 129 - protein, 10 aa, synthetic construct
  REGION 1..10: note = Synthetic
RASSSVSYMN

SEQ ID NO: 130 - protein, 7 aa, synthetic construct
  REGION 1..7: note = Synthetic
DTSKVAS

SEQ ID NO: 131 - protein, 9 aa, synthetic construct
  REGION 1..9: note = Synthetic
QQWSSNPLT

SEQ ID NO: 132 - DNA, 774 bp, synthetic construct
  misc_feature 1..774: note = Synthetic
  source 1..774: mol_type = other DNA
ggtggcggctcaggtggaggccaggtgcagctggtgcagagcggcgcggaagtgaaaaaaccgggcgcgagcgtgaaagtgagctgcaaagcgagcggctatacctttacccgctataccatgcattgggtgcgccaggcgccgggccagggcctggaatggatgggctatattaacccgagccgcggctataccagctatgcgcagaaatttcagggccgcgtgaccatgaccaccgataaaagcaccagcaccgtgtatatggaactgagcagcctgcgcagcgaagataccgcggtgtattattgcgcgcgctattatgatgatcattattgcctggattattggggccagggcaccaccgtgaccgtgagcagcgtggagggcggcagcggcggcagcggcggcagcggcggcagcggcggcgtggacgatattcagatgacccagagcccgagcagcctgagcgcgagcgtgggcgatcgcgtgaccattacctgccgcgcgagcagcagcgtgagctatatgaactggtatcagcagaaaccgggcaaagcgccgaaactgctgatttatgataccagcaaagtggcgagcggcgtgccgagccgctttagcggcagcggcagcggcaccgattataccctgaccattagcagcctgcagccggaagattttgcgacctattattgccagcagtggagcagcaacccgctgacctttggcggcggcaccaaagtggaaattaaacgccaccaccaccaccaccactga

SEQ ID NO: 133 - protein, 257 aa, synthetic construct
  REGION 1..257: note = Synthetic
GGGSGGGQVQLVQSGAEVKKPGASVKVSCKASGYTFTRYTMHWVRQAPGQGLEWMGYINPSRGYTSYAQKFQGRVTMTTDKSTSTVYMELSSLRSEDTAVYYCARYYDDHYCLDYWGQGTTVTVSSVEGGSGGSGGSGGSGGVDDIQMTQSPSSLSASVGDRVTITCRASSSVSYMNWYQQKPGKAPKLLIYDTSKVASGVPSRFSGSGSGTDYTLTISSLQPEDFATYYCQQWSSNPLTFGGGTKVEIKRHHHHHH

SEQ ID NO: 134 - DNA, 642 bp, synthetic construct
  misc_feature 1..642: note = Synthetic
  source 1..642: mol_type = other DNA
gatattcagatgacccagagcccgagcagcctgagcgcgagcgtgggcgatcgcgtgaccattacctgccgcgcgagcagcagcgtgagctatatgaactggtatcagcagaaaccgggcaaagcgccgaaactgctgatttatgataccagcaaagtggcgagcggcgtgccgagccgctttagcggcagcggcagcggcaccgattataccctgaccattagcagcctgcagccggaagattttgcgacctattattgccagcagtggagcagcaacccgctgacctttggcggcggcaccaaagtggaaattaaacgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgctga

SEQ ID NO: 135 - protein, 213 aa, synthetic construct
  REGION 1..213: note = Synthetic
DIQMTQSPSSLSASVGDRVTITCRASSSVSYMNWYQQKPGKAPKLLIYDTSKVASGVPSRFSGSGSGTDYTLTISSLQPEDFATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 136 - DNA, 651 bp, synthetic construct
  misc_feature 1..651: note = Synthetic
  source 1..651: mol_type = other DNA
caggtgcagctggtgcagagcggcgcggaagtgaaaaaaccgggcgcgagcgtgaaagtgagctgcaaagcgagcggctatacctttacccgctataccatgcattgggtgcgccaggcgccgggccagggcctggaatggatgggctatattaacccgagccgcggctataccagctatgcgcagaaatttcagggccgcgtgaccatgaccaccgataaaagcaccagcaccgtgtatatggaactgagcagcctgcgcagcgaagataccgcggtgtattattgcgcgcgctattatgatgatcattattgcctggattattggggccagggcaccaccgtgaccgtgagcagcgcctccaccaaaggtccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagtaacaccaaggtggacaagacagtt

SEQ ID NO: 137 - protein, 217 aa, synthetic construct
  REGION 1..217: note = Synthetic
QVQLVQSGAEVKKPGASVKVSCKASGYTFTRYTMHWVRQAPGQGLEWMGYINPSRGYTSYAQKFQGRVTMTTDKSTSTVYMELSSLRSEDTAVYYCARYYDDHYCLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 138 - DNA, 765 bp, synthetic construct
  misc_feature 1..765: note = Synthetic
  source 1..765: mol_type = other DNA
ggcggtggatcacaggtgcagctggtgcagagcggcgcggaagtgaaaaaaccgggcgcgagcgtgaaagtgagctgcaaagcgagcggctatacctttacccgctataccatgcattgggtgcgccaggcgccgggccagggcctggaatggatgggctatattaacccgagccgcggctataccagctatgcgcagaaatttcagggccgcgtgaccatgaccaccgataaaagcaccagcaccgtgtatatggaactgagcagcctgcgcagcgaagataccgcggtgtattattgcgcgcgctattatgatgatcattattgcctggattattggggccagggcaccaccgtgaccgtgagcagcgtggagggcggcagcggcggcagcggcggcagcggcggcagcggcggcgtggacgatattcagatgacccagagcccgagcagcctgagcgcgagcgtgggcgatcgcgtgaccattacctgccgcgcgagcagcagcgtgagctatatgaactggtatcagcagaaaccgggcaaagcgccgaaactgctgatttatgataccagcaaagtggcgagcggcgtgccgagccgctttagcggcagcggcagcggcaccgattataccctgaccattagcagcctgcagccggaagattttgcgacctattattgccagcagtggagcagcaacccgctgacctttggcggcggcaccaaagtggaaattaaacgccaccaccaccaccaccactga

SEQ ID NO: 139 - protein, 254 aa, synthetic construct
  REGION 1..254: note = Synthetic
GGGSQVQLVQSGAEVKKPGASVKVSCKASGYTFTRYTMHWVRQAPGQGLEWMGYINPSRGYTSYAQKFQGRVTMTTDKSTSTVYMELSSLRSEDTAVYYCARYYDDHYCLDYWGQGTTVTVSSVEGGSGGSGGSGGSGGVDDIQMTQSPSSLSASVGDRVTITCRASSSVSYMNWYQQKPGKAPKLLIYDTSKVASGVPSRFSGSGSGTDYTLTISSLQPEDFATYYCQQWSSNPLTFGGGTKVEIKRHHHHHH

SEQ ID NO: 140 - DNA, 672 bp, synthetic construct
  misc_feature 1..672: note = Synthetic
  source 1..672: mol_type = other DNA
gatattcagatgacccagagcccgagcagcctgagcgcgagcgtgggcgatcgcgtgaccattacctgccgcgcgagcagcagcgtgagctatatgaactggtatcagcgtaaaccgggcaaagcgccgaaactgctgatttatgataccagcaaagtggcgagcggcgtgccgagccgctttagcggcagcggcagcggcaccgattataccctgaccattagcagcctgcagccggaagattttgcgacctattattgccagcagtggagcagcaacccgctgacctttggcggcggcaccaaagtggaaattaaacgcaccgtggcagcacctagcgtgttcatctttcccccttccgacgagcagctgaagtccggcacagcctctgtggtgtgcctgctgaacaatttctatccacgcgaggccaaggtgcagtggaaggtggataacgccctgcagtctggcaatagccaggagtccgtgaccgagcaggactctaaggatagcacatactccctgtcctctaccctgacactgagcaaggccgattacgagaagcacaagctgtatgcatgcgaggtgacccaccagggcctgagctccccagtgacaaagtcttttaaccggggcgagtgtggcggcggctcccaccaccaccaccaccactga

SEQ ID NO: 141 - protein, 223 aa, synthetic construct
  REGION 1..223: note = Synthetic
DIQMTQSPSSLSASVGDRVTITCRASSSVSYMNWYQRKPGKAPKLLIYDTSKVASGVPSRFSGSGSGTDYTLTISSLQPEDFATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECGGGSHHHHHH

SEQ ID NO: 142 - DNA, 1350 bp, synthetic construct
  misc_feature 1..1350: note = Synthetic
  source 1..1350: mol_type = other DNA
caggtgcagctggtgcagagcggcgcggaagtgaaaaaaccgggcgcgagcgtgaaagtgagctgcaaagcgagcggctatacctttacccgctataccatgcattgggtgcgctatgcgccgggccagggcctggaatggatgggctatattaacccgagccgcggctataccagctatgcgcagaaatttcagggccgcgtgaccatgaccaccgataaaagcaccagcaccgtgtatatggaactgagcagcctgcgcagcgaagataccgcggtgtattattgcgcgcgctattatgatgatcattattgcctggattattggggccagggcaccaccgtgaccgtgagcagcgcctctacaaagggacctagcgtgttcccactggcaccctcctctaagtccacctctggaggaacagccgccctgggatgcctggtgaaggattacttcccagagcccgtgaccgtgtcctggaactctggcgccctgaccagcggagtgcacacatttcctgccgtgctgcagagctccggcctgtactccctgtctagcgtggtgacagtgccatcctctagcctgggcacccagacatatatctgcaacgtgaatcacaagccaagcaataccaaggtcgacaagaaggtggagcccaagtcctgtgataagacccacacatgccccccttgtcctgcaccagagctgctgggaggaccaagcgtgttcctgtttccacccaagcctaaggacaccctgatgatctctcggaccccagaggtgacatgcgtggtggtggacgtgagccacgaggatcccgaggtgaagtttaactggtacgtggatggcgtggaggtgcacaatgccaagaccaagcccagggaggagcagtacaattccacctatcgcgtggtgtctgtgctgacagtgctgcaccaggactggctgaacggcaaggagtataagtgcaaggtgtccaataaggccctgcccgcccctatcgagaagacaatctctaaggcaaagggacagcctcgggagccacaggtgtacaccctgcctccatccagagatgagctgaccaagaaccaggtgtctctgacatgtctggtgaagggcttctatccctctgacatcgccgtggagtgggagagcaatggccagcctgagaacaattacgataccacaccccctgtgctggacagcgatggctccttctttctgtatagcgacctgacagtggataagtccagatggcagcagggcaacgtgtttagctgttccgtgatgcacgaggccctgcacaatcactacacccagaagtctctgagcctgtcccccggcaagtaa

SEQ ID NO: 143 - protein, 449 aa, synthetic construct
  REGION 1..449: note = Synthetic
QVQLVQSGAEVKKPGASVKVSCKASGYTFTRYTMHWVRYAPGQGLEWMGYINPSRGYTSYAQKFQGRVTMTTDKSTSTVYMELSSLRSEDTAVYYCARYYDDHYCLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK